(19) United States
(12) Patent Application Publication
(10) Pub. No.: US 2026/0221263 A1
(43) Pub. Date: Jul. 30, 2026

(54) METHODS AND SYSTEMS THAT PLAN AND PROVIDE COST-EFFECTIVE, TIME-EFFICIENT, AND SAFE THERAPEUTIC TREATMENTS TO PATIENTS

(71) Applicant: Craig Alan Friedman, New York, NY (US)

(72) Inventor: Craig Alan Friedman, New York, NY (US)

(21) Appl. No.: 19/452,365

(22) Filed: Jan. 18, 2026

Related U.S. Application Data
(63) Continuation-in-part of application No. 19/374,655, filed on Oct. 30, 2025.
(60) Provisional application No. 63/713,877, filed on Oct. 30, 2024.

(51) Int. Cl. G16H 20/70

(52) U.S. Cl. CPC G16H 20/70

(57) ABSTRACT

The current document is directed to methods and systems that plan and provide cost-effective, time-efficient, and safe therapeutic treatments to patients by generating personalized treatment and therapy plans for patients, including transcranial direct current stimulation (“tDCS”) and transcranial magnetic stimulation (“TMS”). Currently disclosed implementations of these methods and systems incorporate whole-brain network models that are based, in part, on coupled differential equations. Model parameters are adjusted to simulate a patient's brain. Using results of a specified and generally limited number of treatment-application experiments, the currently disclosed methods and systems refine the whole-brain network model to accurately predict treatment efficacies in order to plan optimal or near-optimal treatments.

### CROSS-REFERENCE TO RELATED APPLICATIONS

This application is a continuation-in-part of patent application Ser. No. 19/374,655, filed Oct. 30, 2025, which claims the benefit of Provisional Patent Application 63/713,877, filed Oct. 30, 2024, the contents of which is hereby expressly incorporated by reference in its entirety.

### TECHNICAL FIELD

The current document is directed to methods and systems that plan and provide cost-effective, time-efficient, and safe therapeutic treatments to patients and, in particular, methods and systems that apply brain stimulation to treat neurological and psychiatric conditions, including transcranial direct current stimulation (“tDCS”) and transcranial magnetic stimulation (“TMS”).

### BACKGROUND

There are many different types of treatments and therapies provided to patients suffering from many different types of diseases, pathologies, and disorders. Therapies and treatments may include application of heat and cold, electromagnetic radiation, mechanical forces, and other forces to all or portions of patients' bodies, provision of information and feedback to patients through various means of communication, provision of pharmaceuticals that are ingested, received by injection, inhaled, or delivered to patients by various additional means, surgical interventions, and many other types of therapies. Medical therapies and treatments, including pharmaceuticals, are often thoroughly tested for efficacy and safety before they are allowed to be administered to patients. However, much of this testing is statistical in nature and does not reflect the particular and specific characteristics of individual patients. During the past several decades, it has become increasingly clear that each human being is genetically unique and that medical therapies deemed safe and effective for patients in general may vary considerably in effectiveness and safety among individual patients. These realizations, combined with rapidly evolving technologies for sequencing genomes and acquiring detailed molecular and physiological characterizations of individual patients, have resulted in increasing efforts to personalize medical diagnosis and medical therapies. However, despite significant efforts expended to develop and commercialize personalized medicine, personalized medicine remains largely in the early stages of development and application. In particular, for many types of treatments and therapies, the complexities of evaluating the safety and efficacy of the treatments and therapies with respect to individual patients has rendered many of the current approaches to personalized medicine impractical or infeasible. Medical researchers, medical providers, pharmaceutical developers and manufacturers, and developers of therapy-delivering medical systems and methods therefore continue to seek different and effective approaches to providing personalized therapies to patients.

### SUMMARY

The current document is directed to methods and systems that plan and provide cost-effective, time-efficient, and safe therapeutic treatments to patients by generating personalized treatment and therapy plans for patients, including transcranial direct current stimulation (“tDCS”) and transcranial magnetic stimulation (“TMS”). Currently disclosed implementations of these methods and systems incorporate whole-brain network models that are based, in part, on coupled differential equations. Model parameters are adjusted to simulate a patient's brain. Using results of a specified and generally limited number of treatment-application experiments, the currently disclosed methods and systems refine the whole-brain network model to accurately predict treatment efficacies in order to plan optimal or near-optimal treatments.

### BRIEF DESCRIPTION OF THE DRAWINGS

FIG. 1 provides a general architectural diagram for various types of computers.

FIG. 2 illustrates an Internet-connected distributed computing system.

FIG. 3 illustrates cloud computing.

FIG. 4 illustrates generalized hardware and software components of a general-purpose computer system, such as a general-purpose computer system having an architecture similar to that shown in FIG. 1.

FIGS. 5-6 illustrate two types of virtual machine and virtual-machine execution environments.

FIG. 7 illustrates the cerebral cortex.

FIG. 8 shows a representation of a pyramidal neuron.

FIG. 9 illustrates two canonical neurons and a synaptic connection between them.

FIGS. 10A-D illustrate an action potential.

FIG. 11 shows a representation of a portion of the Brainnetome Atlas.

FIGS. 12A-C illustrate the electroencephalography (“EEG”) method for detecting and recording electrical activity within the brain.

FIG. 13 illustrates application of direct-current transcranial stimulation (“tDCS”) to a patient.

FIG. 14 illustrates application of transcranial magnetic stimulation (“TMS”) to a patient.

FIGS. 15A-C illustrate the interval transform referred to as “convolution.”

FIGS. 16A-C illustrate the Jansen-Rit neural mass model.

FIG. 17 illustrates an application of the ADAM optimization method.

FIGS. 18A-F illustrate Bayesian optimization.

FIGS. 19A-B illustrate the Kuramoto model for synchronization among multiple coupled oscillators.

FIG. 20 illustrates fundamental components of a feed-forward neural network.

FIGS. 21A-J illustrate operation of a very small, example neural network.

FIGS. 22A-C show details of the computation of weight adjustments made by neural-network nodes during backpropagation of error vectors into neural networks.

FIGS. 23A-B illustrate neural-network training.

FIGS. 24A-F illustrate a matrix-operation-based batch method for neural-network training.

FIGS. 25A-C illustrate various aspects of recurrent neural networks.

FIGS. 26A-C illustrate a convolutional neural network.

FIGS. 27A-B provide a control-flow diagram for a routine “treatment determination,” which illustrates significant features of the currently disclosed methods and systems.

FIG. 28 illustrates general concepts and certain notation related to the whole-brain network model (“WBNM”).

FIG. 29 lists additional WBNM model parameters and provides the coupled differential equations that together comprise the basis of the WBNM.

FIG. 30 provides details for the connectivity terms connⱼ and connₗ^{SC} in FIG. 29.

FIG. 31 illustrates the phase dynamics incorporated into the above-discussed connectivity terms.

FIGS. 32A-B provide control-flow diagrams for a routine “simulate EEG” that generates simulated EEG signals observed for a patient for whom a WBNM has been generated.

FIGS. 33A-B illustrate calibration, or optimization, of the WBNM related to step 2710 in FIG. 27A.

FIGS. 34A-C illustrate modification of the optimized or calibrated WBNM* to produce a more accurate modified model, mWBNM, that generates simulated EEG signals under a null stimulation, but that exactly matches the patient's observed EEG under non-stimulating conditions.

FIG. 35 illustrates one implementation of the convolutional neural network that implements the example severity-level function disclosed in the current document.

FIG. 36 shows a simple control-flow diagram illustrating the steps taken to prepare data for input to the convolutional neural network used to determine the severity level associated with simulated or observed EEG signals.

FIG. 37 illustrates bandpass filtering.

FIGS. 38A-E illustrate severity-level determination.

FIG. 39 provides a control-flow diagram for a routine “refinement.”

FIG. 40 illustrates determination of the change in severity level following therapeutic treatment.

FIGS. 41A-B illustrate one implementation of model_{in_silico} and model_{in_vivo}.

FIG. 42 illustrates the deformation or modification of a model to produce a model that incorporates additional patient information gleaned from limited treatment experimentation.

FIGS. 43A-B illustrate the deformation process introduced above with reference to FIG. 42.

FIG. 44 illustrates a technique used to expand the search space of control-variable vectors explored in the constrained optimization/minimization processes discussed above with reference to FIGS. 43A-B.

FIG. 45 illustrates a few modifications to the WBNM that can be used to adapt a WBNM to other therapeutic methods.

### DETAILED DESCRIPTION

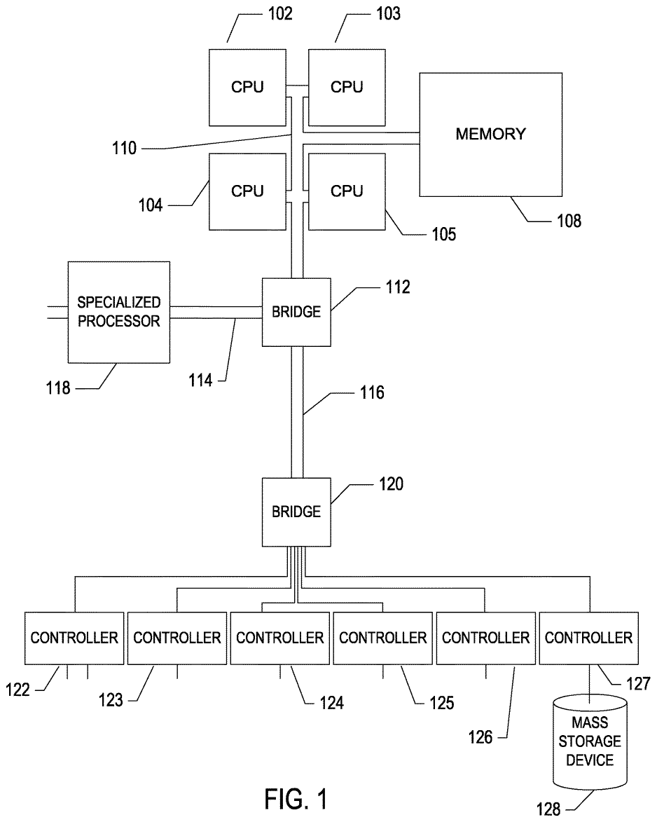

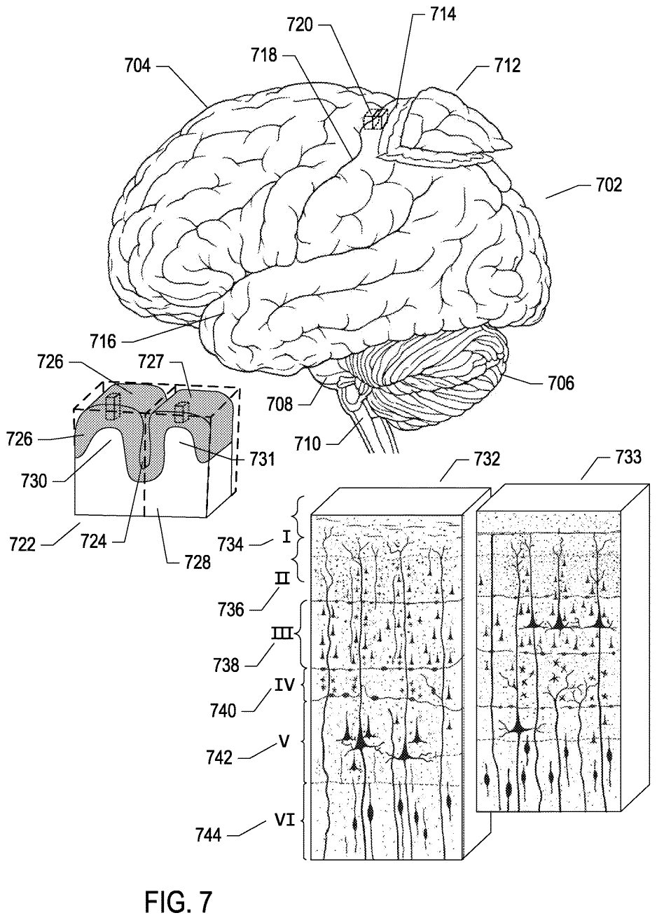

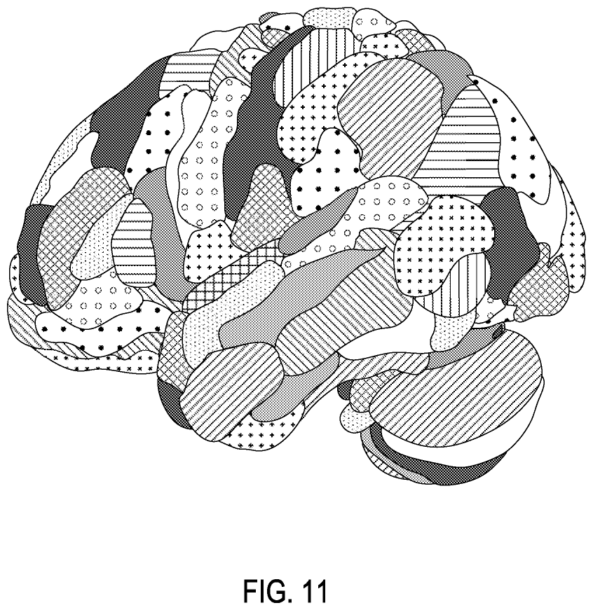

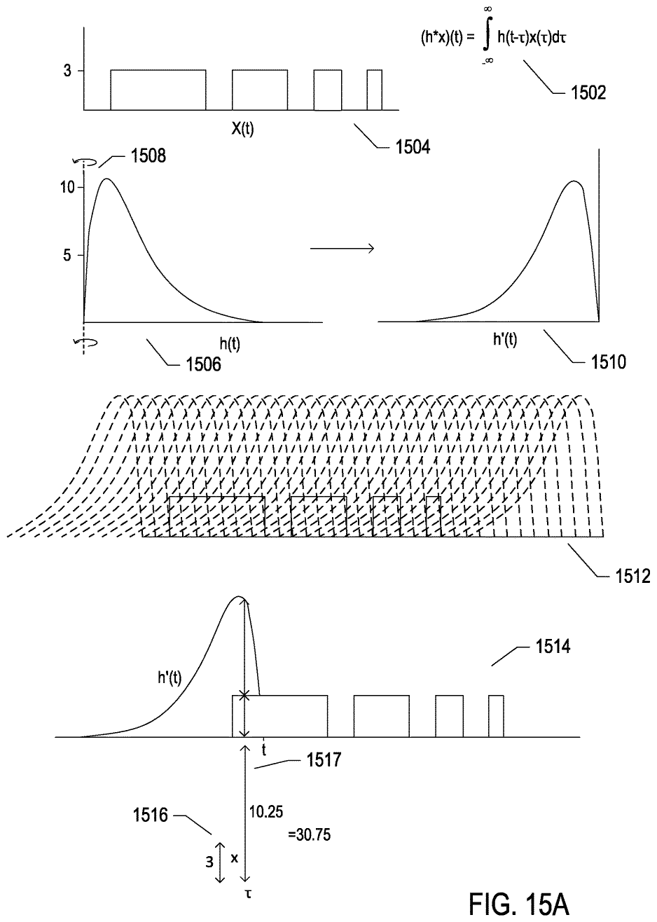

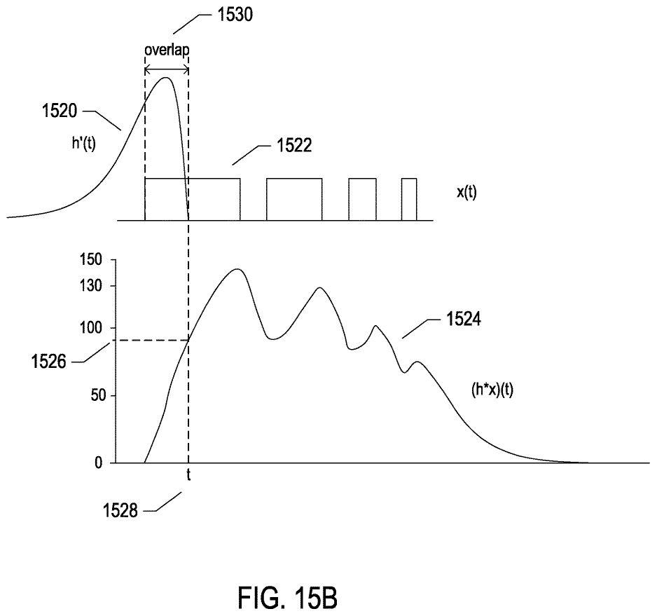

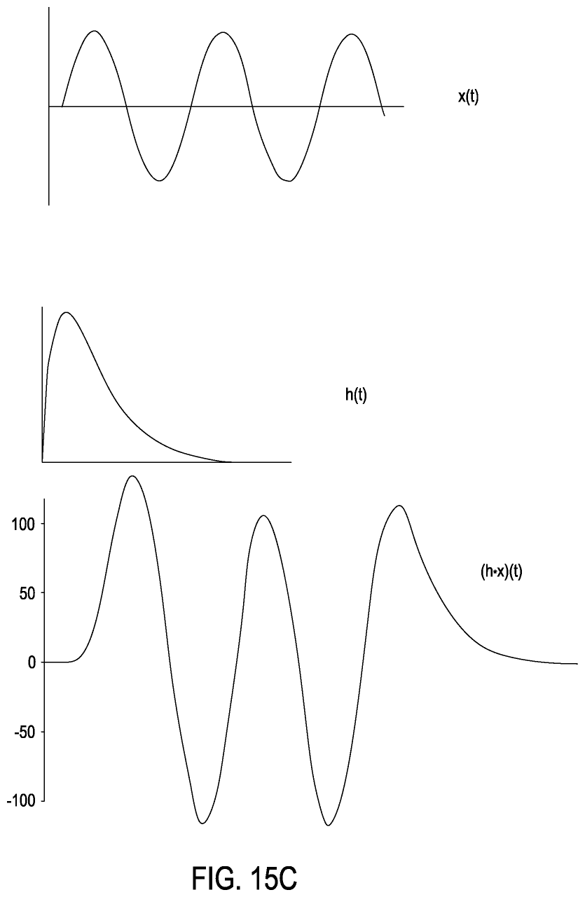

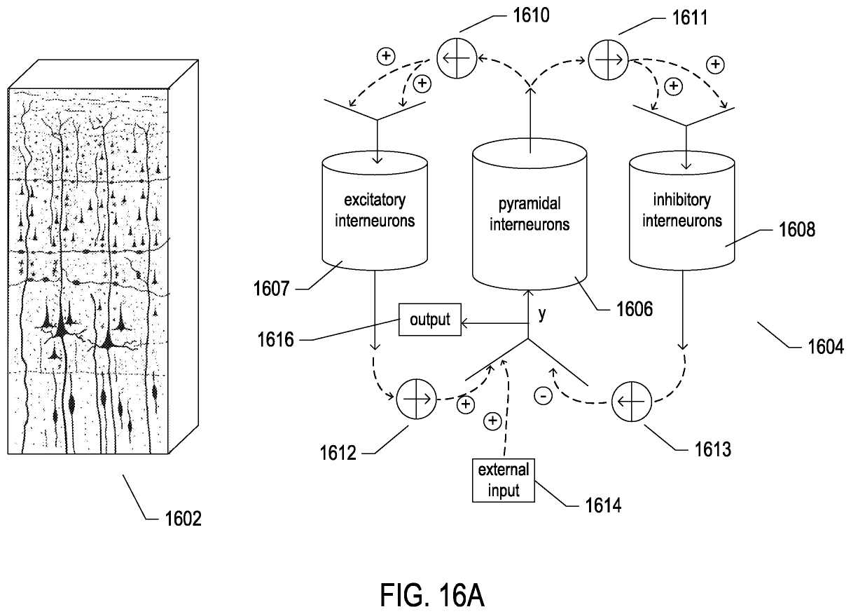

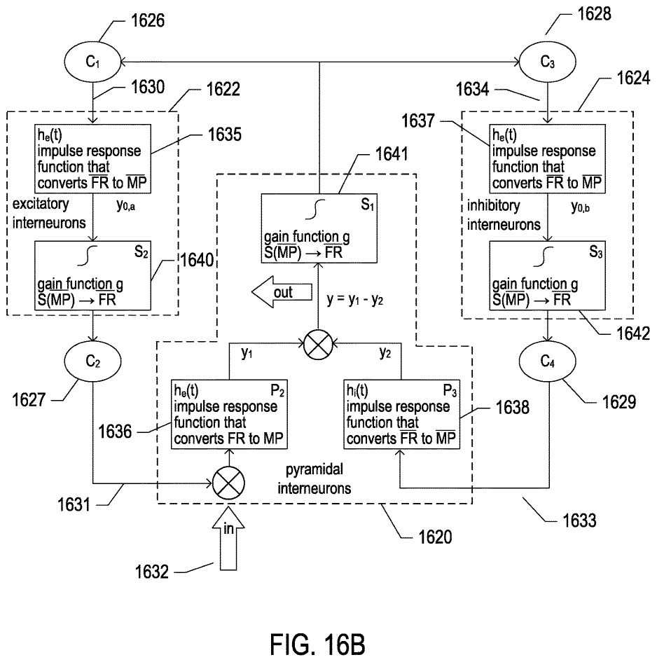

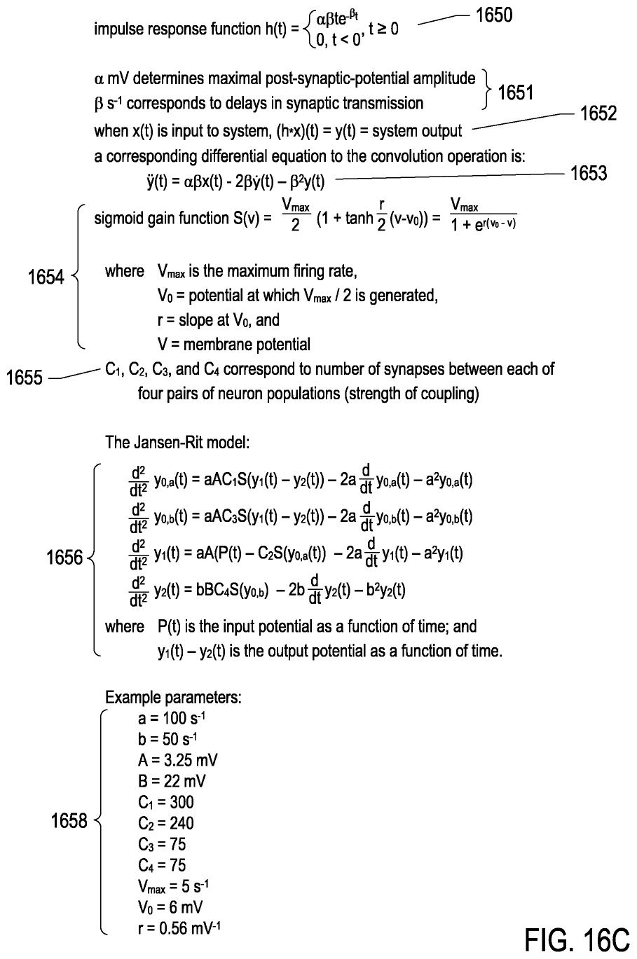

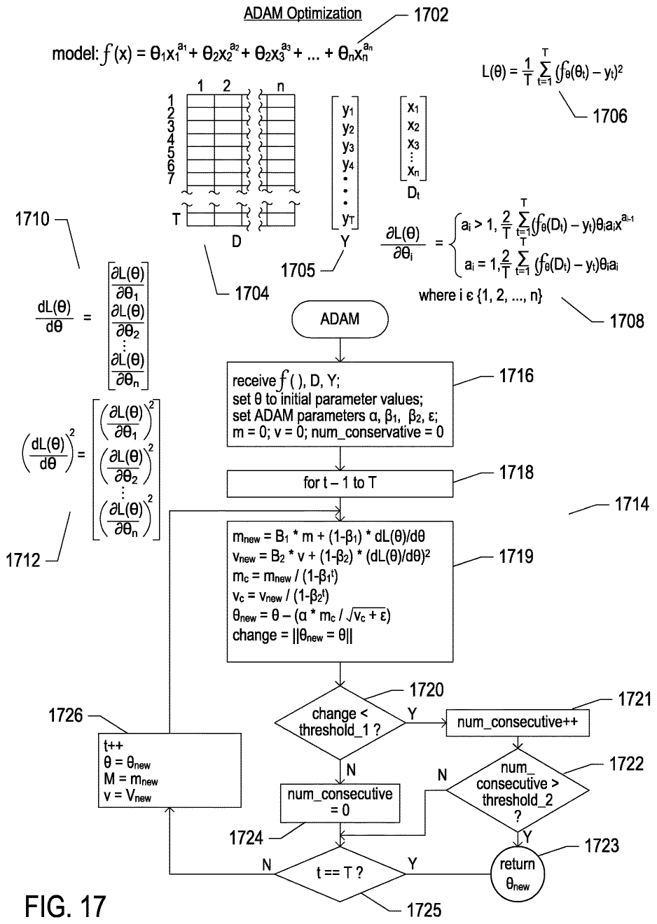

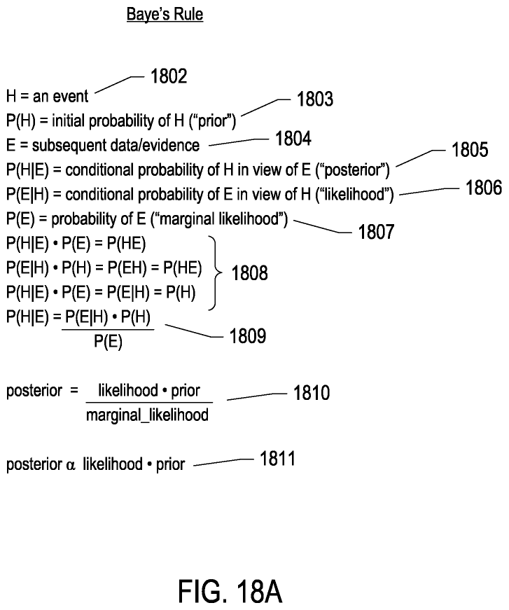

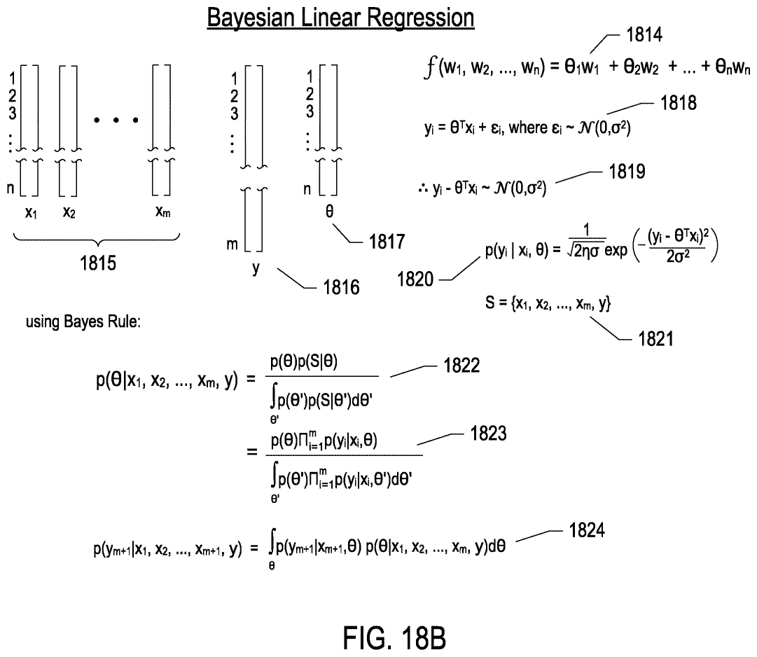

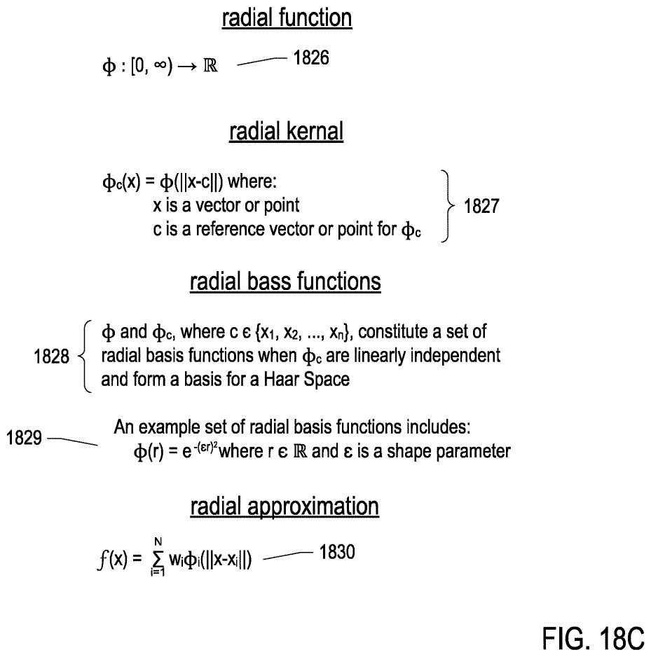

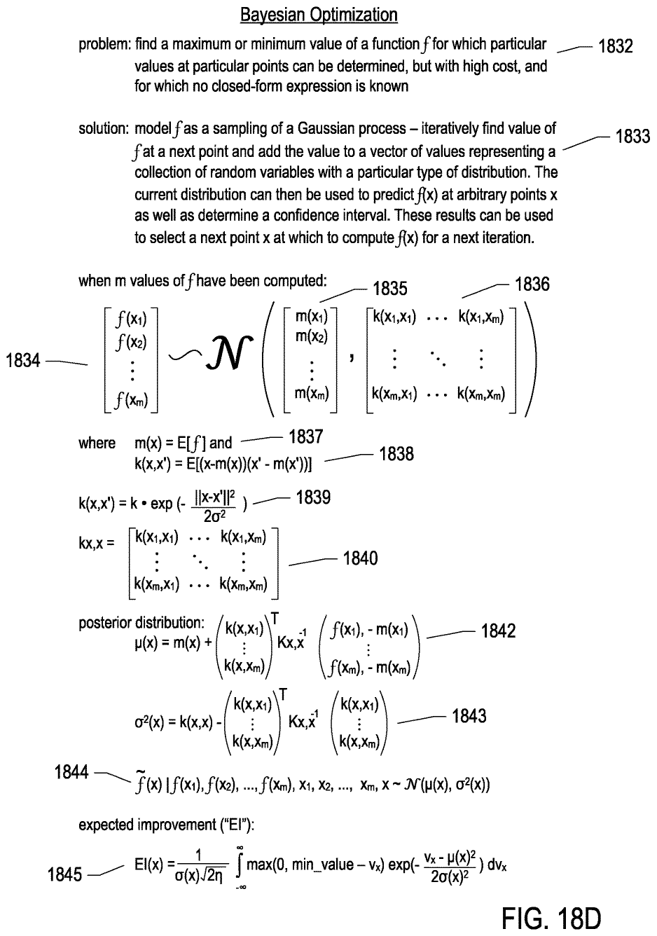

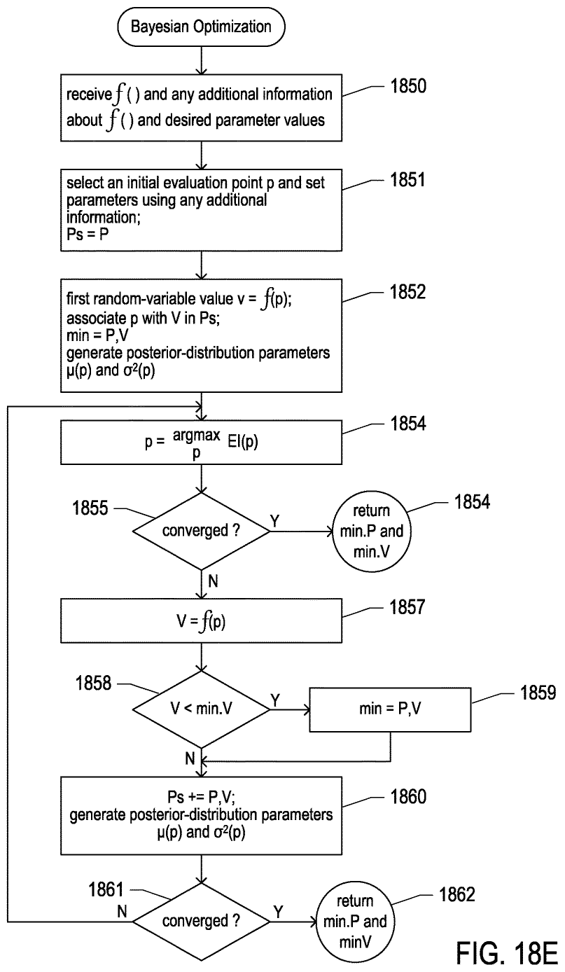

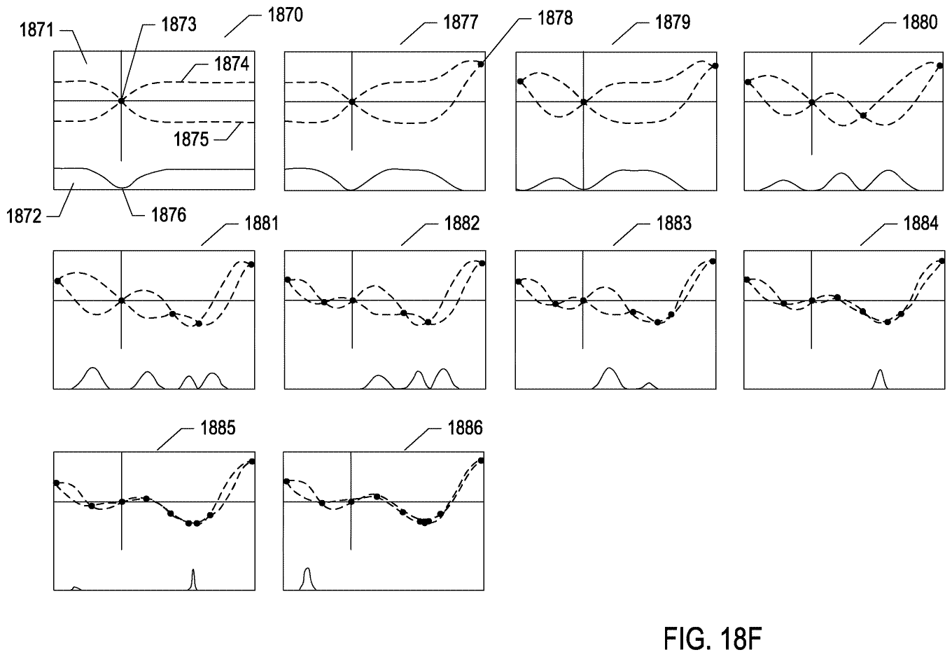

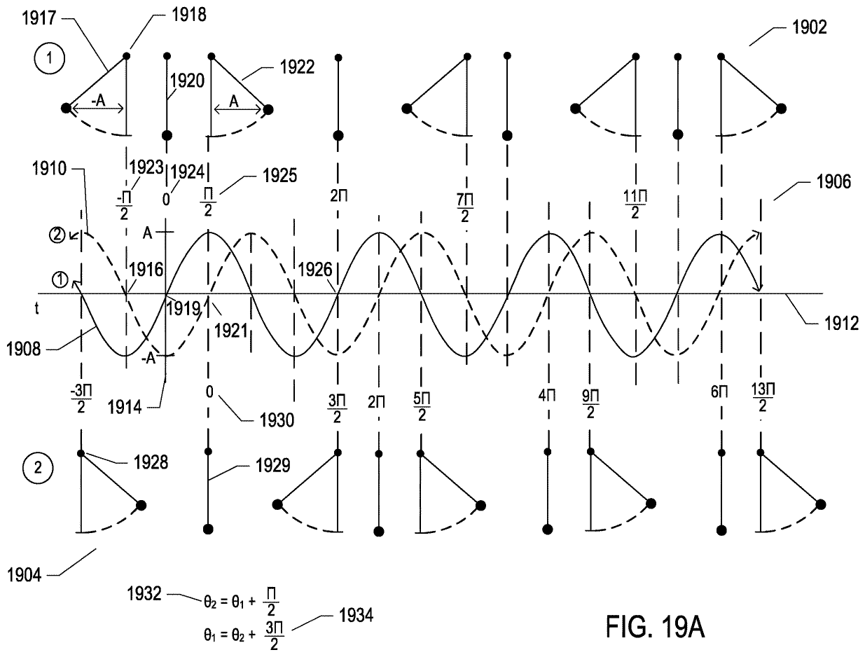

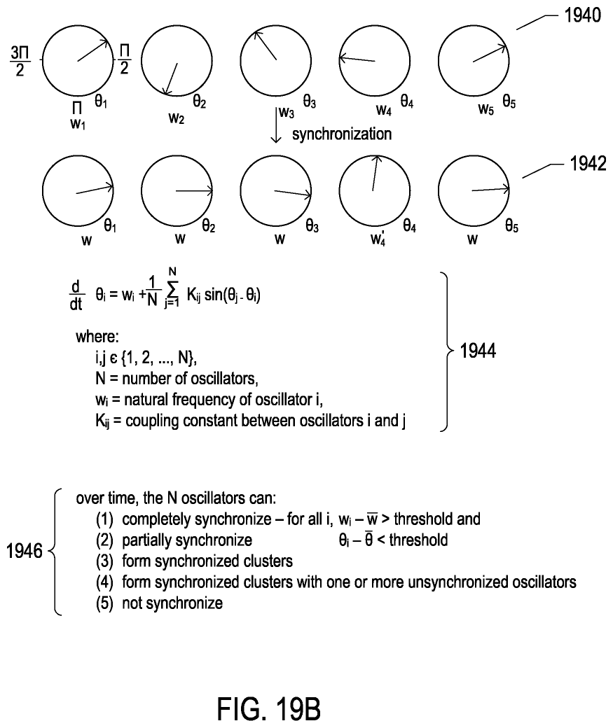

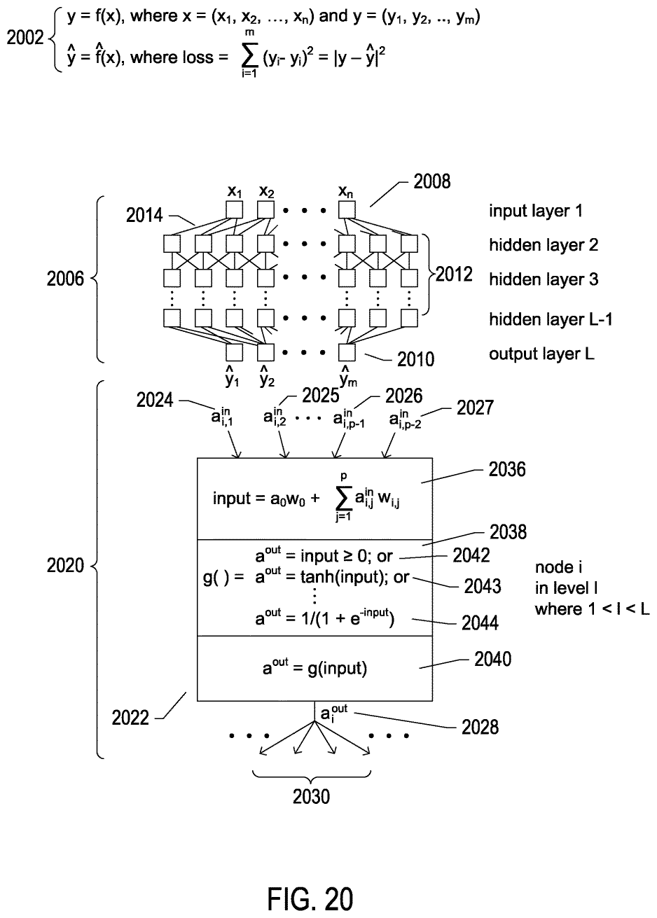

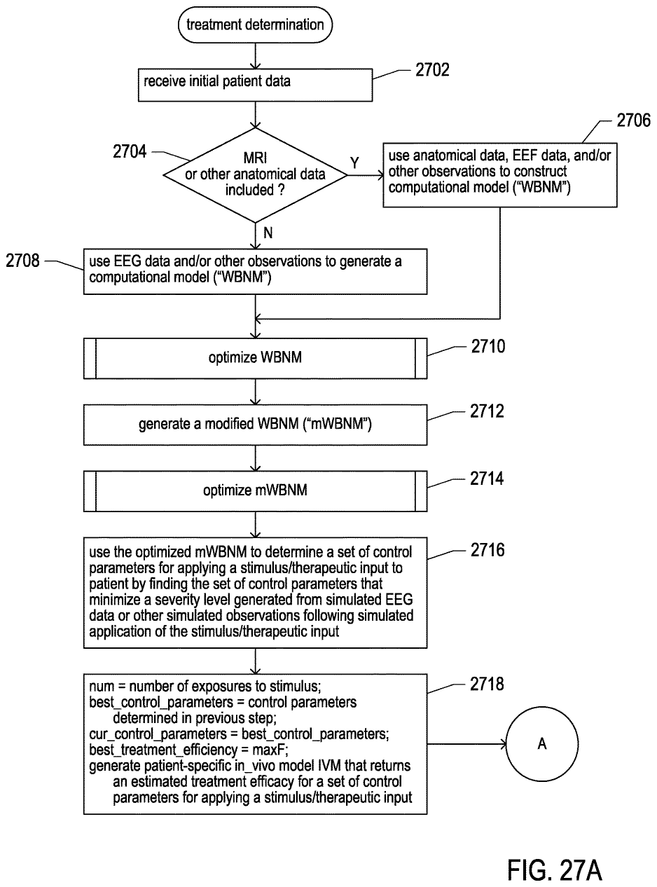

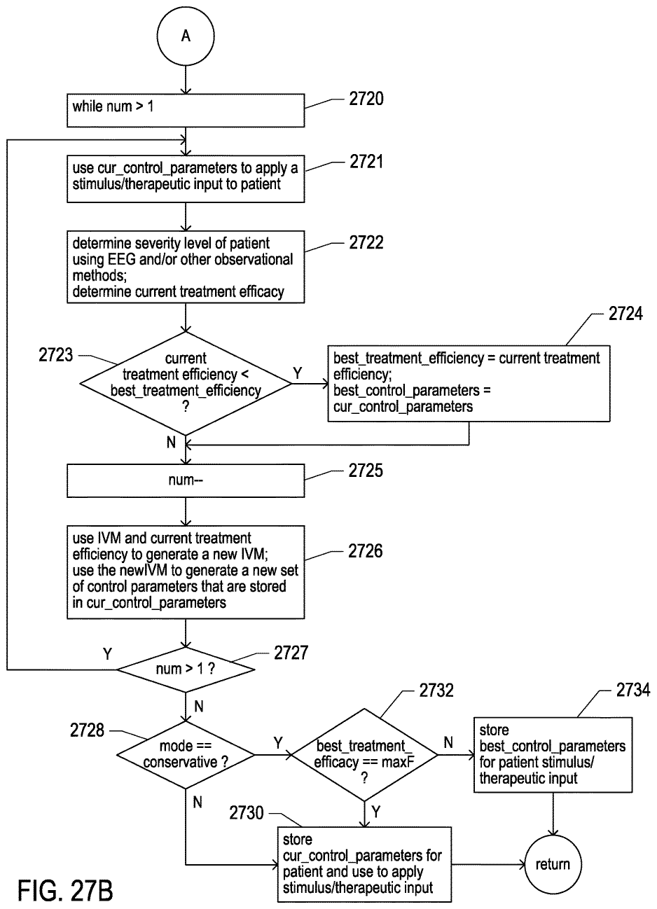

The current document is directed to methods and systems that plan and apply personalized treatments to patients, including transcranial direct current stimulation (“tDCS”) and transcranial magnetic stimulation (“TMS”). A first subsection provides an overview of computer hardware, complex computational systems, operating systems, and virtualization with reference to FIGS. 1-6. A second subsection provides an overview of relevant aspects of brain anatomy and function, with reference to FIGS. 7-10D. A third subsection provides an overview of brain-region models, electroencephalography (“EEG”), transcranial direct-current stimulation (“tDCS”), and transcranial magnetic stimulation (“TMS”), with reference to FIGS. 11-14. A fourth subsection provides an overview of the convolution operation, with reference to FIGS. 15A-C. A fifth subsection provides an overview of the Jansen-Ritt neural mass model, with reference to FIGS. 16A-C. A sixth subsection provides an overview of the adaptive moment estimation (“ADAM”) and Bayesian optimization techniques, with reference to FIGS. 17-18F. A seventh subsection provides an overview of the Kuramoto synchronization model, with reference to FIGS. 19A-B. An eighth subsection provides an overview of neural networks, with reference to FIGS. 20-26C. The currently disclosed methods and systems are discussed in a final subsection, with reference to FIGS. 27A-45.

### Computer Hardware, Complex Computational Systems, and Virtualization

Computational entities, such as programs, routines, interfaces, and executables, are tangible, physical entities and interfaces that are implemented in physical computer hardware, data-storage devices, and communications systems. One frequently encounters assertions that, because a computational system is described in terms of abstractions, functional layers, interfaces, programs, and routines, the computational system is somehow different from a physical machine or device. One also frequently encounters statements that characterize a computational technology as being “only software,” and thus not a machine or device. Software is essentially a sequence of encoded symbols, such as a printout of a computer program or digitally encoded computer instructions sequentially stored in a file on an optical disk or within an electromechanical mass-storage device or data-storage appliance. Software alone can do nothing. It is only when encoded computer instructions are loaded into an electronic memory within a computer system and executed on a physical processor that so-called “software implemented” functionality is provided. The digitally encoded computer instructions are an essential and physical control component of processor-controlled machines and devices, no less essential and physical than a cam-shaft control system in an internal-combustion engine.

FIG. 1 provides a general architectural diagram for various types of computers, including computers that execute currently disclosed methods for brain simulation, severity-level determination, treatment planning, and controlling treatment-providing devices and systems. The computer system contains one or multiple central processing units (“CPUs”) 102-105, one or more electronic memories 108 interconnected with the CPUs by a CPU/memory-subsystem bus 110 or multiple busses, a first bridge 112 that interconnects the CPU/memory-subsystem bus 110 with additional busses 114 and 116, or other types of high-speed interconnection media, including multiple, high-speed serial interconnects. These busses or serial interconnections, in turn, connect the CPUs and memory with specialized processors, such as a graphics processor 118, and with one or more additional bridges 120, which are interconnected with high-speed serial links or with multiple controllers 122-127, such as controller 127, that provide access to various different types of mass-storage devices 128, electronic displays, input devices, and other such components, subcomponents, and computational resources. It should be noted that computer-readable data-storage devices include optical and electromagnetic disks, electronic memories, and other physical data-storage devices. Those familiar with modern science and technology appreciate that electromagnetic radiation and propagating signals do not store data for subsequent retrieval and can transiently “store” only a byte or less of information per mile, far less information than needed to encode even the simplest of routines.

Of course, there are many different types of computer-system architectures that differ from one another in the number of different memories, including different types of hierarchical cache memories, the number of processors and the connectivity of the processors with other system components, the number of internal communications busses and serial links, and in many other ways. However, computer systems generally execute stored programs by fetching instructions from memory and executing the instructions in one or more processors. Computer systems include general-purpose computer systems, such as personal computers (“PCs”), various types of servers and workstations, and higher-end mainframe computers, but may also include a plethora of various types of special-purpose computing devices, including data-storage systems, communications routers, network nodes, tablet computers, and mobile telephones.

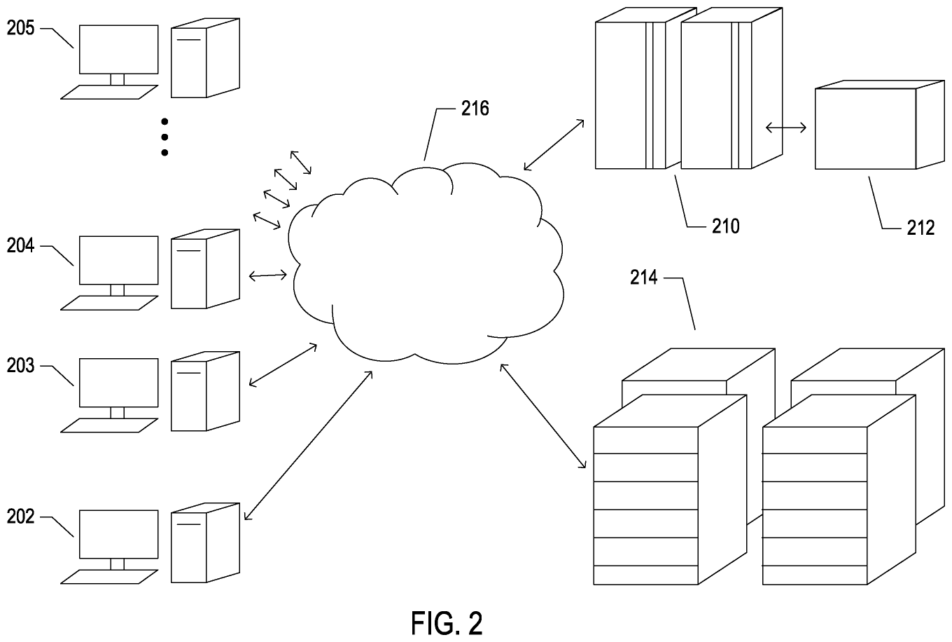

FIG. 2 illustrates an Internet-connected distributed computing system. As communications and networking technologies have evolved in capability and accessibility, and as the computational bandwidths, data-storage capacities, and other capabilities and capacities of various types of computer systems have steadily and rapidly increased, much of modern computing now generally involves large distributed systems and computers interconnected by local networks, wide-area networks, wireless communications, and the Internet. FIG. 2 shows a typical distributed system in which a large number of PCs 202-205, a high-end distributed mainframe system 210 with a large data-storage system 212, and a large computer center 214 with large numbers of rack-mounted servers or blade servers all interconnected through various communications and networking systems that together comprise the Internet 216. Such distributed computing systems provide diverse arrays of functionalities. For example, a PC user sitting in a home office may access hundreds of millions of different web sites provided by hundreds of thousands of different web servers throughout the world and may access high-computational-bandwidth computing services from remote computer facilities for running complex computational tasks.

Until recently, computational services were generally provided by computer systems and data centers purchased, configured, managed, and maintained by service-provider organizations. For example, an e-commerce retailer generally purchased, configured, managed, and maintained a data center including numerous web servers, back-end computer systems, and data-storage systems for serving web pages to remote customers, receiving orders through the web-page interface, processing the orders, tracking completed orders, and other myriad different tasks associated with an e-commerce enterprise.

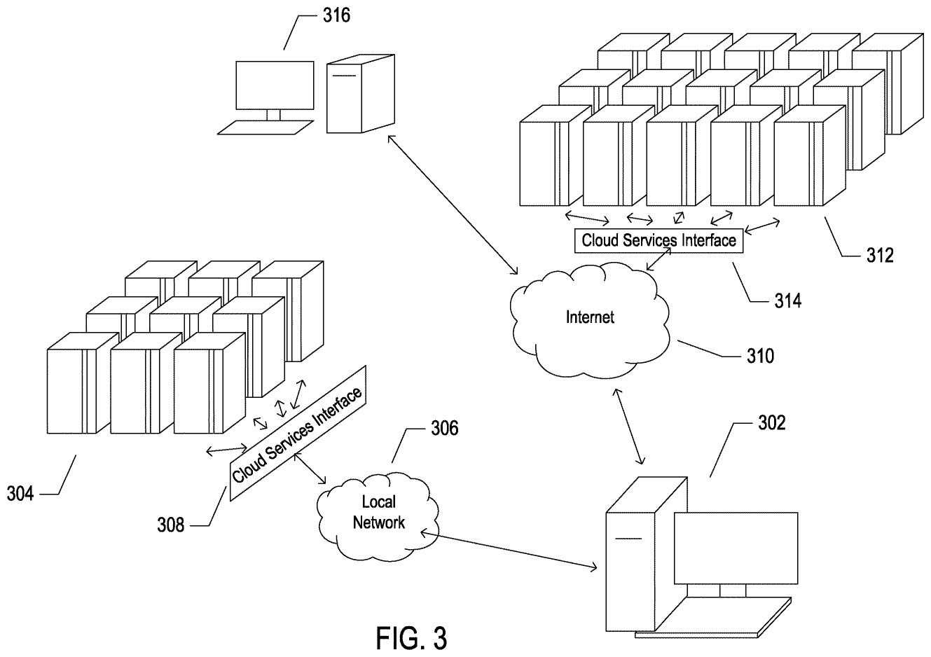

FIG. 3 illustrates cloud computing. In the recently developed cloud-computing paradigm, computing cycles and data-storage facilities are provided to organizations and individuals by cloud-computing providers. In addition, larger organizations may elect to establish private cloud-computing facilities in addition to, or instead of, subscribing to computing services provided by public cloud-computing service providers. In FIG. 3, a system administrator for an organization, using a PC 302, accesses the organization's private cloud 304 through a local network 306 and private-cloud interface 308 and also accesses, through the Internet 310, a public cloud 312 through a public-cloud services interface 314. The administrator can, in either the case of the private cloud 304 or public cloud 312, configure virtual computer systems and even entire virtual data centers and launch execution of application programs on the virtual computer systems and virtual data centers in order to carry out any of many different types of computational tasks. As one example, a small organization may configure and run a virtual data center within a public cloud that executes web servers to provide an e-commerce interface through the public cloud to remote customers of the organization, such as a user viewing the organization's e-commerce web pages on a remote user system 316.

Cloud-computing facilities are intended to provide computational bandwidth and data-storage services much as utility companies provide electrical power and water to consumers. Cloud computing provides enormous advantages to small organizations without the resources to purchase, manage, and maintain in-house data centers. Such organizations can dynamically add and delete virtual computer systems from their virtual data centers within public clouds in order to track computational-bandwidth and data-storage needs, rather than purchasing sufficient computer systems within a physical data center to handle peak computational-bandwidth and data-storage demands. Moreover, small organizations can completely avoid the overhead of maintaining and managing physical computer systems, including hiring and periodically retraining information-technology specialists and continuously paying for operating-system and database-management-system upgrades. Furthermore, cloud-computing interfaces allow for easy and straightforward configuration of virtual computing facilities, flexibility in the types of applications and operating systems that can be configured, and other functionalities that are useful even for owners and administrators of private cloud-computing facilities used by a single organization.

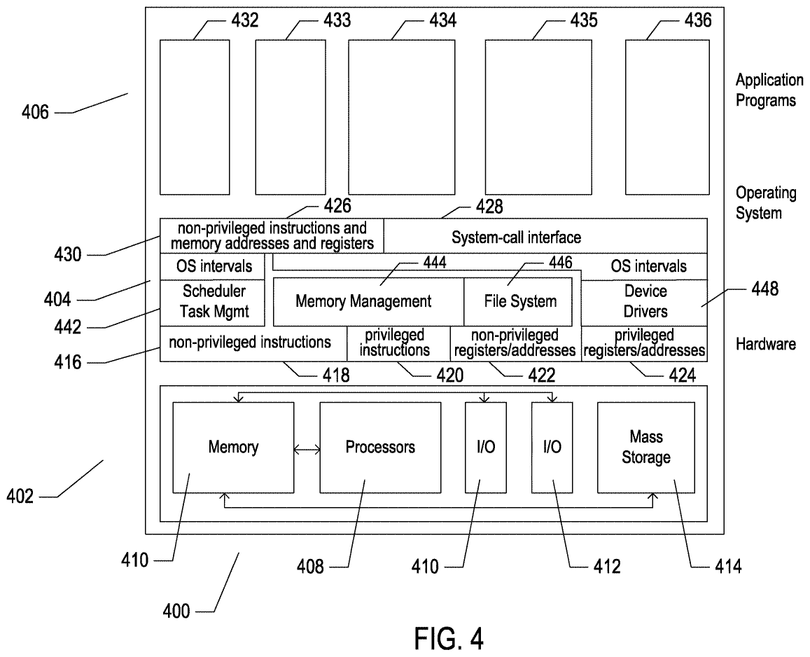

FIG. 4 illustrates generalized hardware and software components of a general-purpose computer system, such as a general-purpose computer system having an architecture similar to that shown in FIG. 1. The computer system 400 is often considered to include three fundamental layers: (1) a hardware layer or level 402; (2) an operating-system layer or level 404; and (3) an application-program layer or level 406. The hardware layer 402 includes one or more processors 408, system memory 410, various different types of input-output (“I/O”) devices 410 and 412, and mass-storage devices 414. Of course, the hardware level also includes many other components, including power supplies, internal communications links and busses, specialized integrated circuits, many different types of processor-controlled or microprocessor-controlled peripheral devices and controllers, and many other components. The operating system 404 interfaces to the hardware level 402 through a low-level operating system and hardware interface 416 generally comprising a set of non-privileged computer instructions 418, a set of privileged computer instructions 420, a set of non-privileged registers and memory addresses 422, and a set of privileged registers and memory addresses 424. In general, the operating system exposes non-privileged instructions, non-privileged registers, and non-privileged memory addresses 426 and a system-call interface 428 as an operating-system interface 430 to application programs 432-436 that execute within an execution environment provided to the application programs by the operating system. The operating system, alone, accesses the privileged instructions, privileged registers, and privileged memory addresses. By reserving access to privileged instructions, privileged registers, and privileged memory addresses, the operating system can ensure that application programs and other higher-level computational entities cannot interfere with one another's execution and cannot change the overall state of the computer system in ways that could deleteriously impact system operation. The operating system includes many internal components and modules, including a scheduler 442, memory management 444, a file system 446, device drivers 448, and many other components and modules. To a certain degree, modern operating systems provide numerous levels of abstraction above the hardware level, including virtual memory, which provides to each application program and other computational entities a separate, large, linear memory-address space that is mapped by the operating system to various electronic memories and mass-storage devices. The scheduler orchestrates interleaved execution of various different application programs and higher-level computational entities, providing to each application program a virtual, stand-alone system devoted entirely to the application program. From the application program's standpoint, the application program executes continuously without concern for the need to share processor resources and other system resources with other application programs and higher-level computational entities. The device drivers abstract details of hardware-component operation, allowing application programs to employ the system-call interface for transmitting and receiving data to and from communications networks, mass-storage devices, and other I/O devices and subsystems. The file system 436 facilitates abstraction of mass-storage-device and memory resources as a high-level, easy-to-access, file-system interface. Thus, the development and evolution of the operating system has resulted in the generation of a type of multi-faceted virtual execution environment for application programs and other higher-level computational entities.

While the execution environments provided by operating systems have proved to be an enormously successful level of abstraction within computer systems, the operating-system-provided level of abstraction is nonetheless associated with difficulties and challenges for developers and users of application programs and other higher-level computational entities. One difficulty arises from the fact that there are many different operating systems that run within various different types of computer hardware. In many cases, popular application programs and computational systems are developed to run on only a subset of the available operating systems and can therefore be executed within only a subset of the various different types of computer systems on which the operating systems are designed to run. Often, even when an application program or other computational system is ported to additional operating systems, the application program or other computational system can nonetheless run more efficiently on the operating systems for which the application program or other computational system was originally targeted. Another difficulty arises from the increasingly distributed nature of computer systems. Although distributed operating systems are the subject of considerable research and development efforts, many of the popular operating systems are designed primarily for execution on a single computer system. In many cases, it is difficult to move application programs, in real time, between the different computer systems of a distributed computing system for high-availability, fault-tolerance, and load-balancing purposes. The problems are even greater in heterogeneous distributed computing systems which include different types of hardware and devices running different types of operating systems. Operating systems continue to evolve, as a result of which certain older application programs and other computational entities may be incompatible with more recent versions of operating systems for which they are targeted, creating compatibility issues that are particularly difficult to manage in large distributed systems.

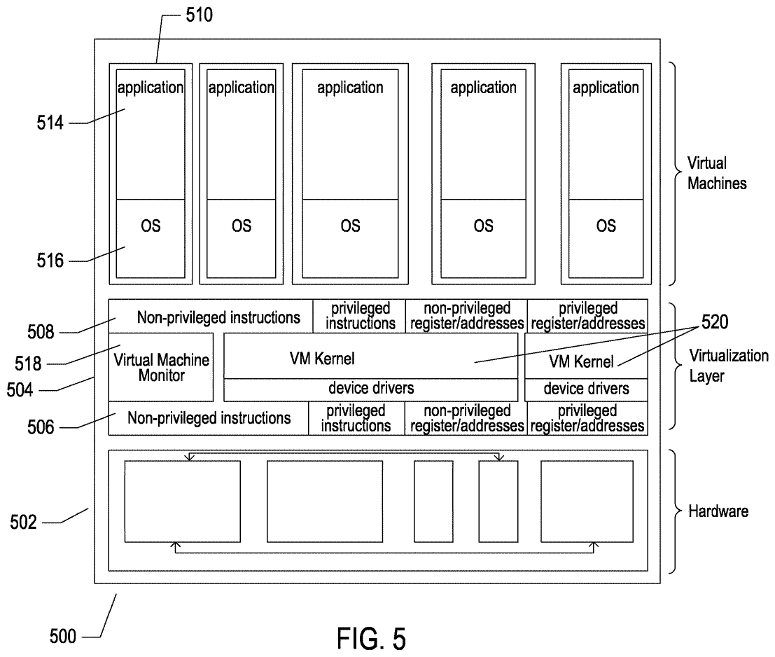

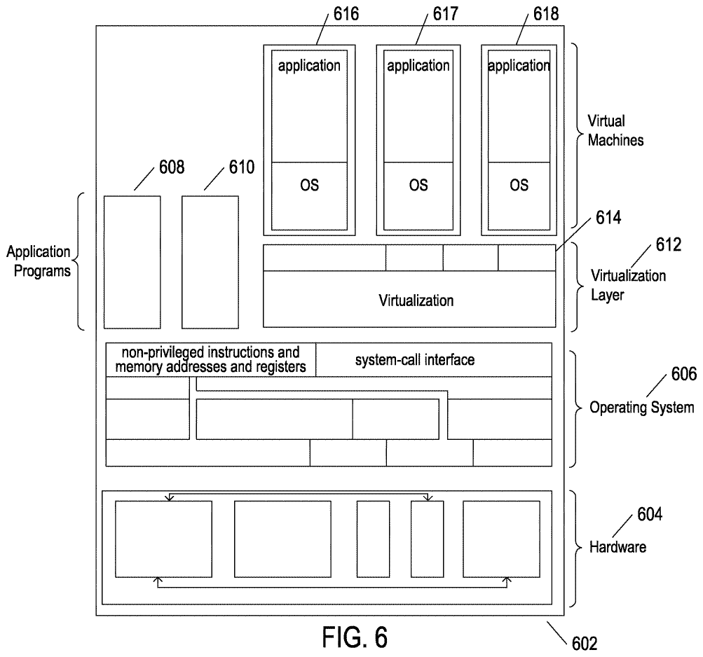

For all of these reasons, a higher level of abstraction, referred to as the “virtual machine,” has been developed and evolved to further abstract computer hardware in order to address many difficulties and challenges associated with traditional computing systems, including the compatibility issues discussed above. FIGS. 5-6 illustrate several types of virtual machine and virtual-machine execution environments. FIGS. 5-6 use the same illustration conventions as used in FIG. 4. FIG. 5 shows a first type of virtualization. The computer system 500 in FIG. 5A includes the same hardware layer 502 as the hardware layer 402 shown in FIG. 4. However, rather than providing an operating system layer directly above the hardware layer, as in FIG. 4, the virtualized computing environment illustrated in FIG. 5A features a virtualization layer 504 that interfaces through a virtualization-layer/hardware-layer interface 506, equivalent to interface 416 in FIG. 4, to the hardware. The virtualization layer provides a hardware-like interface 508 to a number of virtual machines, such as virtual machine 510, executing above the virtualization layer in a virtual-machine layer 512. Each virtual machine includes one or more application programs or other higher-level computational entities packaged together with an operating system, referred to as a “guest operating system,” such as application 514 and guest operating system 516 packaged together within virtual machine 510. Each virtual machine is thus equivalent to the operating-system layer 404 and application-program layer 406 in the general-purpose computer system shown in FIG. 4. Each guest operating system within a virtual machine interfaces to the virtualization-layer interface 508 rather than to the actual hardware interface 506. The virtualization layer partitions hardware resources into abstract virtual-hardware layers to which each guest operating system within a virtual machine interfaces. The guest operating systems within the virtual machines, in general, are unaware of the virtualization layer and operate as if they were directly accessing a true hardware interface. The virtualization layer ensures that each of the virtual machines currently executing within the virtual environment receive a fair allocation of underlying hardware resources and that all virtual machines receive sufficient resources to progress in execution. The virtualization-layer interface 508 may differ for different guest operating systems. For example, the virtualization layer is generally able to provide virtual hardware interfaces for a variety of different types of computer hardware. This allows, as one example, a virtual machine that includes a guest operating system designed for a particular computer architecture to run on hardware of a different architecture. The number of virtual machines need not be equal to the number of physical processors or even a multiple of the number of processors.

The virtualization layer includes a virtual-machine-monitor module 518 (“VMM”) that virtualizes physical processors in the hardware layer to create virtual processors on which each of the virtual machines executes. For execution efficiency, the virtualization layer attempts to allow virtual machines to directly execute non-privileged instructions and to directly access non-privileged registers and memory. However, when the guest operating system within a virtual machine accesses virtual privileged instructions, virtual privileged registers, and virtual privileged memory through the virtualization-layer interface 508, the accesses result in execution of virtualization-layer code to simulate or emulate the privileged resources. The virtualization layer additionally includes a kernel module 520 that manages memory, communications, and data-storage machine resources on behalf of executing virtual machines (“VM kernel”). The VM kernel, for example, maintains shadow page tables on each virtual machine so that hardware-level virtual-memory facilities can be used to process memory accesses. The VM kernel additionally includes routines that implement virtual communications and data-storage devices as well as device drivers that directly control the operation of underlying hardware communications and data-storage devices. Similarly, the VM kernel virtualizes various other types of I/O devices, including keyboards, optical-disk drives, and other such devices. The virtualization layer essentially schedules execution of virtual machines much like an operating system schedules execution of application programs, so that the virtual machines each execute within a complete and fully functional virtual hardware layer.

FIG. 6 illustrates a second type of virtualization. In FIG. 6, the computer system 602 includes the same hardware layer 604 and operating-system layer 606 as the hardware layer 402 shown in FIG. 4. Several application programs 608 and 610 are shown running in the execution environment provided by the operating system. In addition, a virtualization layer 612 is also provided, in computer 602, but, unlike the virtualization layer 504 discussed with reference to FIG. 5, virtualization layer 612 is layered above the operating system 606, referred to as the “host OS,” and uses the operating system interface to access operating-system-provided functionality as well as the hardware. The virtualization layer 612 comprises primarily a VMM and a hardware-like interface 614, similar to hardware-like interface 508 in FIG. 5. The virtualization-layer/hardware-layer interface 614, equivalent to interface 416 in FIG. 4, provides an execution environment for a number of virtual machines 616-617-618, each including one or more application programs or other higher-level computational entities packaged together with a guest operating system.

The currently disclosed methods are implemented in, and may be distributed across, a variety of different types of computer systems, including virtual-machine-based implementations running in cloud-computing facilities and/or data centers.

### An Overview of Relevant Aspects of Brain Anatomy and Function

FIG. 7 illustrates the cerebral cortex. An external view of the human brain 702 is shown at the top of FIG. 7. The human brain includes the cerebrum 704, consisting of two cerebral hemispheres, the cerebellum 706, the brainstem 708, which connects the brain to the spinal cord 710, and many additional internal regions and components, including four lobes within each cerebral hemisphere, a ventricular system, the thalamus, the epithalamus, the pineal gland, the hypothalamus, the pituitary gland, and an internal vascular system. The brain includes numerous different cell types, with the dominant cell types including neurons and glial cells. There is an estimated average of 86 billion neurons in the human brain and a comparable number of other types of cells. The image of the human brain 702 includes a small cutaway 712 showing the outer layer of the brain 714 in cross-section, with the thickness exaggerated. The outer layer is referred to as the “cerebral cortex.” It contains between 14 and 16 billion neurons and ranges in thickness from about 2 to 4 mm. The cerebral cortex is highly convoluted, with many folds or ridges referred to as “gyri,” such as ridge 716, and grooves between the ridges referred to as “sulci,” such as central sulcus 718. Two dashed, contiguous rectangular volumes 720 are shown at larger scale in representation 722. In this representation, the central sulcus 724 can be seen as a relatively deep invagination of the cerebral cortex between two folds or ridges 726-727. Even though the cerebral cortex is relatively thin, the folded, convoluted structure of the cerebral cortex results in the cerebral cortex having a relatively large surface area of between 2 and 3 ft.². It contains approximately 40% of the mass of the brain. The cerebral cortex includes an outer, thicker layer referred to as the “neocortex” and a thinner, lower layer referred to as the “allocortex.” The neocortex represents 90% of the cerebral cortex.

The cerebral cortex contains many different regions that have been identified to contain neurons, neural circuits, and neuronal networks related to various functionalities, most generally sensory, motor, and association functionalities. Sensory regions receive and process sensory information from various sensory organs of the body, including eyes, ears, the nose, the tongue, the skin, and other body parts and organs that can sense various types of inputs, including mechanical stress and strain, mechanical contact, illumination, chemical inputs, pressure waves, and other such inputs. Motor regions are involved in the control of voluntary movements, including movements of the limbs and fingers. The association areas are related to abstract thinking, perceptual experience, language processing, planning, and abstract thought.

In representation 722, the cerebral cortex is shown as a shaded layer 726 above an unshaded volume 728. In fact, the cerebral cortex is referred to as “gray matter” and forms the outer layer above a volume of white matter within the brain. Two dashed rectangular columns 730-731 are shown at larger scale in columns 732-733. Column 732 represents a cross-section of the primary-motor-cortex region of the cerebral neocortex and column 733 represents a cross-section of the primary-somatosensory-cortex region of the cerebral neocortex. The layers of the neocortex are labeled, alongside column 732, with Roman numerals representing the commonly used Roman-numeral designations of the layers. The first layer 734 is referred to as the “molecular layer,” and consists primarily of extensions of apical dendritic tufts of pyramidal neurons, discussed below, horizontal axons, and glial cells. The first layer receives inputs from external brain regions, including the thalamus, as well as from non-local regions of the neocortex. The second layer 736 includes small pyramidal neurons (discussed below) and other types of neurons. The third layer 738 contains pyramidal neurons and other types of neurons with vertically oriented axons and is an important source of signals directed to external brain regions. The fourth layer 740 includes pyramidal cells and other types of neurons and receives input signals from various external brain regions and other regions of the cerebral cortex. The fifth layer 742 is referred to as the “internal pyramidal layer” and contains large pyramidal neurons with axons projecting into subcortical structures. The sixth layer 744 is referred to as the “polymorphic layer” and contains large pyramidal neurons and smaller pyramidal neurons and multiform neurons and extends efferent nerve fibers to the thalamus. Of course, the various layers include additional types of neurons and cells, including interneurons that interconnect neighboring neurons into neural circuits. The interneurons tend to exhibit inhibitory effects on neurons to which they are connected while the pyramidal neurons tend to exhibit excitatory effects on neurons to which they are connected. A prominent characteristic of the neocortex is that the long apical dendrites emitted from pyramidal neurons extend vertically upward, in parallel, within the neocortex layers imparting a distinct anisotropy to the neocortex.

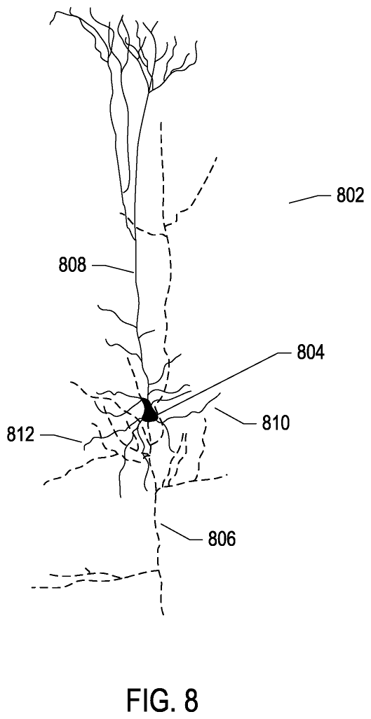

FIG. 8 shows a representation of a pyramidal neuron. A pyramidal neuron 802 includes a cell body 804, a branching axon, represented by dashed curves 806, and an apical dendrite 808 and multiple basal dendrites, including basal dendrites 810 and 812. A typical pyramidal neuron may receive around 30,000 excitatory inputs and 1,700 inhibitory inputs.

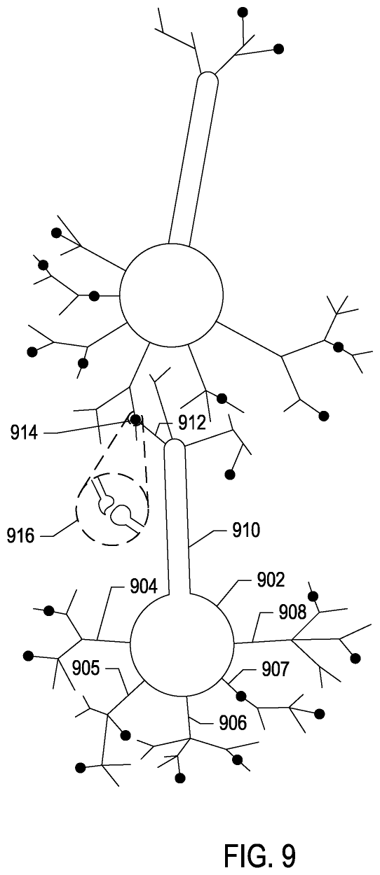

FIG. 9 illustrates two canonical neurons and a synaptic connection between them. There are many different types of neurons with many different numbers and types of features. In general, a neuron has a cell body 902, one or more dendrites 904-908, typically branching, and one or more axons 910 with terminal branches, such as terminal branch 912. Interconnections between neurons are created by synapses, such as synapse 914, shown at larger scale in inset 916. Electrical signals are generally received at dendritic synapses by a neuron and, when sufficient incoming signals have been received to raise the membrane potential of the neuron above a threshold level, results in the neuron generating an output signal, referred to as an “action potential,” described further below. The action potential propagates outward, from the cell body through the axon to the terminal branches of the axon. At the synapse, the action potential opens voltage-gated channels, discussed below, which allow ions, generally calcium ions, to cross through the membrane of the axon terminal branches that result in the release of neurotransmitters from synaptic vessels into the intracellular space between the axon terminal branches of the presynaptic neuron and the dendrites of the postsynaptic neuron. There are many different neurotransmitters, including glutamate, glycine, gamma-amino butyric acid, acetylcholine, serotonin, epinephrine, norepinephrine, dopamine, and adenosine triphosphate (“ATP”), to name a few. When certain of the neurotransmitters bind to certain receptors within the dendrites of the postsynaptic neuron, ligand-gated channels are opened, allowing exchange of ions between the interior of the dendrites of the postsynaptic neuron and the intracellular environment which can, in turn, result in a change in the electric potential across the dendritic membrane. When sufficient incoming signals raise the potential past a threshold level, the incoming signals are propagated forward by the postsynaptic neuron to additional downstream neurons. The effects of different types of neurotransmitters on the postsynaptic neuron can be excitatory, inhibitory, or modulatory, depending on the types of receptors to which the neurotransmitters bind in the postsynaptic dendritic membrane.

The human brain contains an estimated average of 86 billion neurons, each with an estimated average of 7000 synaptic connections to other neurons, resulting in between 1 and 5 trillion synaptic connections. The interconnections between neurons form myriad local neural circuits which, in turn, form a large number of larger neuronal networks that span the brain, spinal cord, and peripheral tissues. The strength of propagating signals is generally related to the rate at which neurons generate action potentials rather than the magnitude of the electric-potential changes. The cooperative behavior of signal generation and propagation within collections of neurons result in many different dynamic patterns of signal transmission, including oscillatory signal propagation. Of course, because of the complexity of the human brain, it is difficult to assign particular logical functionalities to particular signals and signal patterns, but different patterns of electrical transmission within the brain result in controlled movement, sensory perception, thought, and consciousness. Moreover, particular patterns of signal transmission are indicative of various different mental states.

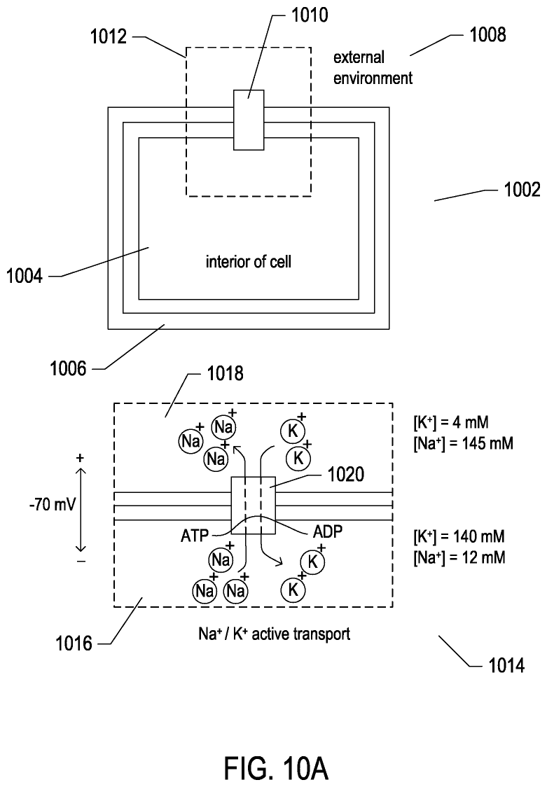

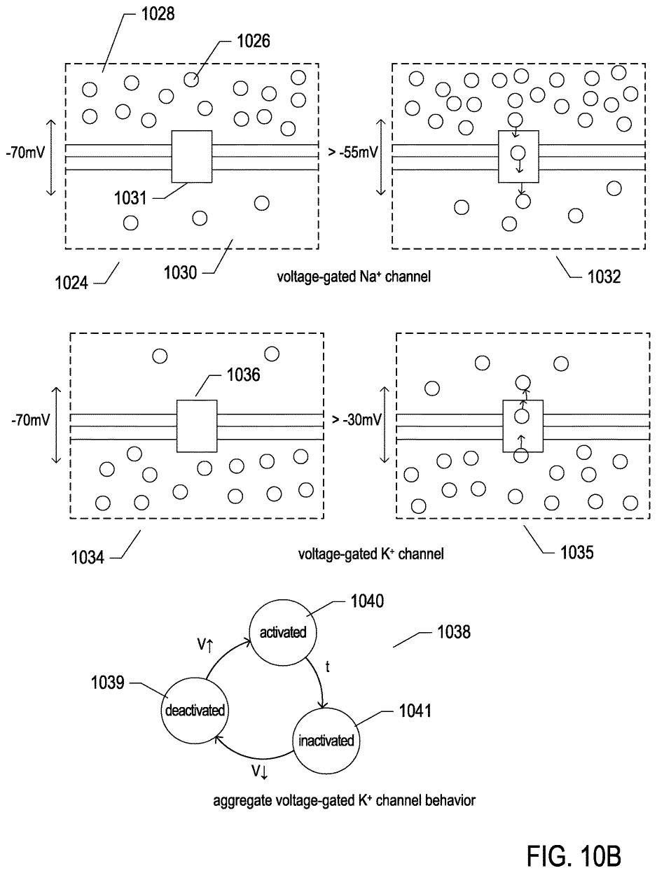

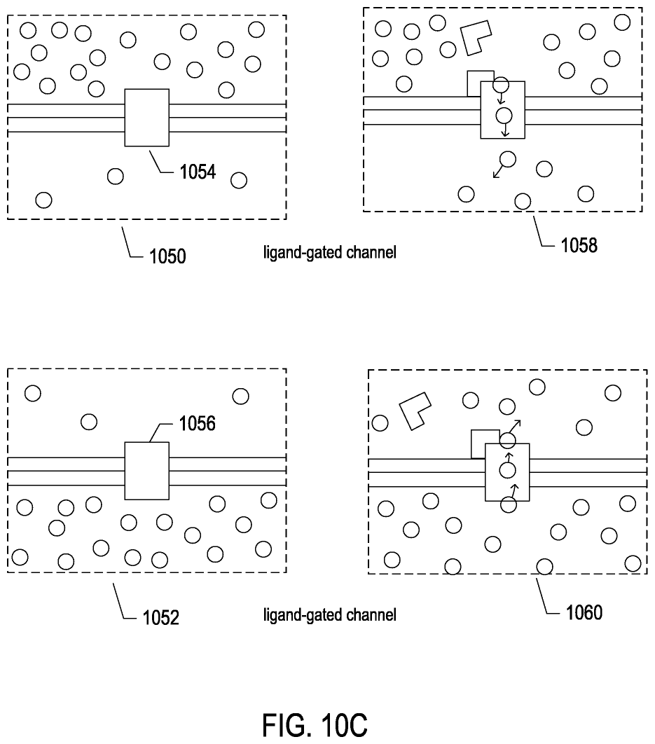

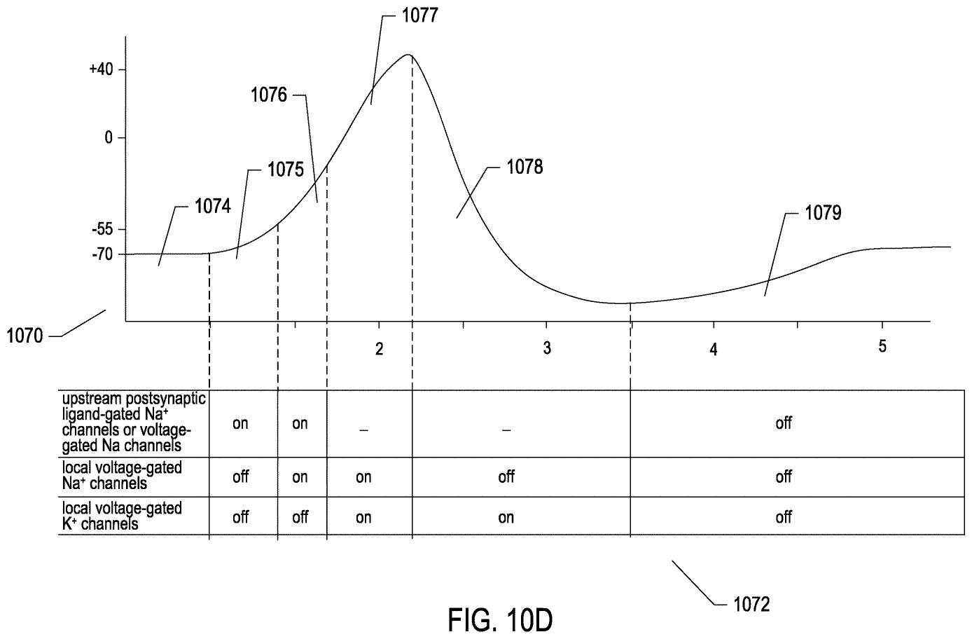

FIGS. 10A-D illustrate an action potential. The illustration conventions used in FIGS. 10A-C are explained with respect to the representation of a cell 1002 shown at the top of FIG. 10A. The cell is represented by an interior volume 1004 enclosed by a lipid-bilayer membrane 1006. The membrane is generally impermeable to charged ions and many small molecules and macromolecules. Thus, the interior of the cell 1004 is chemically isolated from the external environment 1008 of the cell. However, lipid-bilayer membranes include numerous different types of protein complexes, referred to as “transporters” and “channels,” that each spans the lipid-bilayer membrane to provide a pathway for certain ions, small molecules, and/or macromolecules to pass through the lipid-bilayer membrane. Channels generally provide passive transport which allows ions to move down electrochemical gradients across the lipid-bilayer membrane, with the direction of transport depending on the direction of the electrochemical gradients between the cell and its external environment. Transporters, by contrast, are generally unidirectional and often involve consumption of energy, such as hydrolysis of adenosine triphosphate, in order to move ions, small molecules, and/or macromolecules across the lipid-bilayer membrane. In the representation of the cell 1002, the rectangular inclusion 1010 represents a channel or transporter. In the lower portion of FIG. 10A and in FIGS. 10B-C, illustrations are directed to a channel or transporter within a membrane, illustrated within dashed rectangle 1012.

The lower portion 1014 of FIG. 10A illustrates the sodium/potassium transporter primarily responsible for maintaining a resting-state −70 mV electrical gradient across neurons and other cells. The interior 1016 of a neuron is negatively charged relative to the exterior 1018 of the cell. The sodium/potassium transporter 1020 consumes the energy contained in one phosphate bond of ATP to move two potassium ions across the lipid-bilayer membrane into the cell while moving three sodium ions across the lipid-bilayer membrane from the interior of the cell to the exterior. The sodium/potassium transporter thus generates a relatively more positive chemical environment in the exterior of the cell relative to the interior of the cell. A typical cell may have many tens of thousands of transporters and channels that promote passive diffusion and active transport of many different types of ions, molecules, and macromolecules across the lipid-bilayer membrane. The maintenance of the negative voltage gradient and negative polarity of the interior of the cell with respect to the external environment is a result of many complex chemical and biochemical components and processes, but the sodium/potassium transporter plays a significant role.

FIG. 10B illustrates two voltage-gated ion channels. As shown in diagram 1024, due in part to the above-discussed sodium/potassium transporter, there is a −70 mV potential across the lipid-bilayer membrane and a steep concentration gradient with respect to sodium ions across the lipid-bilayer membrane, illustrated by the many more sodium ions, such as sodium ion 1026, in the external environment 1028 than in the cell interior 1030. At the normal negative electrical potential of −70 mV, voltage-gated sodium channels, such as voltage-gated sodium channel 1031, are closed, preventing diffusion of sodium ions through the channel. However, when the normal negative electrical gradient begins to decrease, become more positive, and eventually reaches a threshold level of −55 mV, as shown in diagram 1032, the voltage-gated sodium channels begin to open, allowing diffusion of sodium ions into the cell down the negative electrochemical gradient. Similarly, as shown in diagrams 1034-1035, voltage-gated potassium channels, such as voltage-gated potassium channel 1036, are generally closed at the normal −70 mV membrane potential, but as the cell depolarizes and reaches a threshold level of −30 mV, the voltage-gated potassium channels begin to open and allow for migration of potassium ions from the interior of the cell to the external environment, against the electrical gradient but down the potassium-ion-concentration gradient.

As shown in the small state-transition diagram 1038 at the bottom of FIG. 10B, a neuron generally occupies one of three states: (1) deactivated 1039; (2) activated 1040; and (3) inactivated 1041. In the deactivated state, the cell exhibits the normal −70 mV negative electrical gradient or negative polarization. When, due to depolarization of the cell, sodium channels begin to open and the ratio of opened sodium channels to closed sodium channels increases due to a positive feedback loop, the cell enters the activated state 1040 and the electrical gradient reverses to a potential of +40 mV or a higher, which initiates an action potential comprising a wave-like reversal of the cell polarity that propagates outward along the axon. Once depolarization has reached the threshold of −30 mV for the potassium channels, the potassium channels also open. Once the depolarization has reached +40 mV or higher, the sodium channels close and the cell begins to repolarize due to the migration of potassium ions from the interior of the cell to the external environment through the voltage-gated potassium channels. The repolarization generally continues to a negative electrical potential significantly less than −70 mV, which is referred to as “hyperpolarization.” When the cell is hyperpolarized, it occupies the inactivated state 1041. In the inactivated state, a greater depolarization is required for triggering another action potential. However, over time, the sodium/potassium active transporter reestablishes the normal resting negative electrical gradient of −70 mV, the cell transitions from the inactivated state 1041 back to the deactivated state 1039. The action potential generally arises in a portion of the axon close to the cell body The transition to the inactivated state results in the action potential generally propagating in an outward direction from the cell body towards the axon terminal branches since and sections of the axon that have recently fired have transitioned to the inactivated state and are not easily again triggered to fire while the sections of the axon further away from the cell body are in the deactivated state until the wave of depolarization reaches them.

FIG. 10C illustrates ligand-gated channels. As mentioned above, in the dendritic portions of synapses, neurotransmitters, emitted from the presynaptic neuron, bind to ligand-gated channels to allow ions to move across the dendritic lipid-bilayer membrane and alter the polarization of the postsynaptic neuron. As shown in diagram 1050, in a resting-state postsynaptic neuron, an imbalance of a particular type of ion between the interior of the cell and the exterior of the cell may have been established due to a transporter, such as the sodium/potassium transporter. In diagram 1052, an opposite-polarity imbalance is shown. In both cases, a ligand-gated channel 1054 and 1056 is in a closed state, preventing diffusion of the particular type of iron or ions controlled by the ligand-gated channels across the lipid-bilayer membrane. However, as shown in diagrams 1058 and 1016, binding of a ligand particular to the ligand-gated channels opens the ligand-gated channels and allows ions to flow through the channel down a concentration or electrochemical gradient. Binding of ligands to ligand-gated channels can result in depolarization or hyperpolarization of the postsynaptic neuron, depending on the type of ligand-gated channel to which the ligands bind. Binding of a neurotransmitter to a ligand-gated channel can result in an excitatory input to the postsynaptic neuron, an inhibitory input to the postsynaptic neuron, or a modulatory input to the postsynaptic neuron, depending on the type of ligand-gated channel and the ions or molecules that pass through it when the ligand-gated channel is open. For example, when a neurotransmitter binds to a ligand-gated channel that results in hyperpolarization of the postsynaptic neuron, the hyperpolarization may decrease the chance that a subsequent excitatory input will contribute to the generation of an action potential. By contrast, when a neurotransmitter binds to a ligand-gated channel that results in depolarization of the postsynaptic neuron, the depolarization may contribute to the generation of an action potential once the postsynaptic neuron is sufficiently depolarized to reach the threshold-level polarization state at which sodium channels begin to open.

FIG. 10D illustrates an action potential. Plot 1070 shows a voltage-gradient versus time curve for a portion of the axon of a neuron. In a table aligned below the plot 1072, the on/off states of voltage-gated and/or ligand-gated channels are shown for portions of the voltage/time curve demarcated by dashed vertical lines. In a first region of the curve 1074, the neuron is in a resting state and all of the voltage-gated and ligand-gated channels represented in the aligned table 1072 are closed. In a second region 1075 of the voltage/time curve, upstream postsynaptic ligand-gated channels or voltage-gated sodium channels in an upstream portion of the axon or cell body of the neuron have been opened, contributing to an initial depolarization of the portion of the axon of the neuron. In the third region 1076 of the voltage/time curve, additional depolarization has occurred to the point that the sodium-channel threshold voltage has been reached, at which point the local voltage-gated sodium channels within the axon portion have opened. In the fourth portion of the voltage/time curve 1077, a positive feedback loop results in additional sodium channels opening and further depolarization of the axon portion. The threshold potential for opening of the potassium channels has been reached, and the potassium channels have begun to contribute to repolarization of the axon portion, but that repolarization effect is much smaller than the depolarization effect of the opened sodium channels. In a fifth portion of the voltage/time curve 1078, the peak depolarization is reached, the sodium channels have closed, and the potassium channels remain open and begin to quickly repolarize and then hyperpolarize the axon portion. Finally, in the sixth portion of the voltage/time curve 1079, the hyperpolarization of the axon portion diminishes as the sodium/potassium transporter reestablishes the resting potential of −70 mV.

In summary, various types of electrical signals comprising changing electrical potentials in neurons arise in particular regions of the brain and propagate within those regions as well as between regions. These signals may be impulses, oscillatory, intermittent, and/or exhibit other patterns and forms. They arise from polarization and depolarization of neurons, including dendrites, the neuron cell bodies, and axons. Particularly strong signals are generated within the cerebral cortex due to the long, parallel apical dendrites emanating from pyramidal neurons. These electrical signals correspond to interneuron and inter-brain-region communication, neuronal-network activity, and diverse higher-level types of neurological activity.

### An Overview of Brain-Region Maps, Electroencephalography (“EEG”), Transcranial Direct-Current Stimulation (“tDCS”), and Transcranial Magnetic Stimulation (“TMS”)

FIG. 11 shows a representation of a portion of the Brainnetome Atlas. The Brainnetome Atlas is one of numerous maps that have been developed in order to describe and delineate anatomical regions and associated functionalities along with connections between the various different regions. For example, the Brainnetome Atlas provides a parcellation of the human brain into 246 regions, including 210 cortical regions and 36 subcortical regions. Automated registration techniques can be used to assign Atlas labels to regions of MRI images as well as to align electrodes and other sensors with underlying regions. In FIG. 11, each of multiple different regions is indicated by different shadings, crosshatchings, or other region-filling patterns.

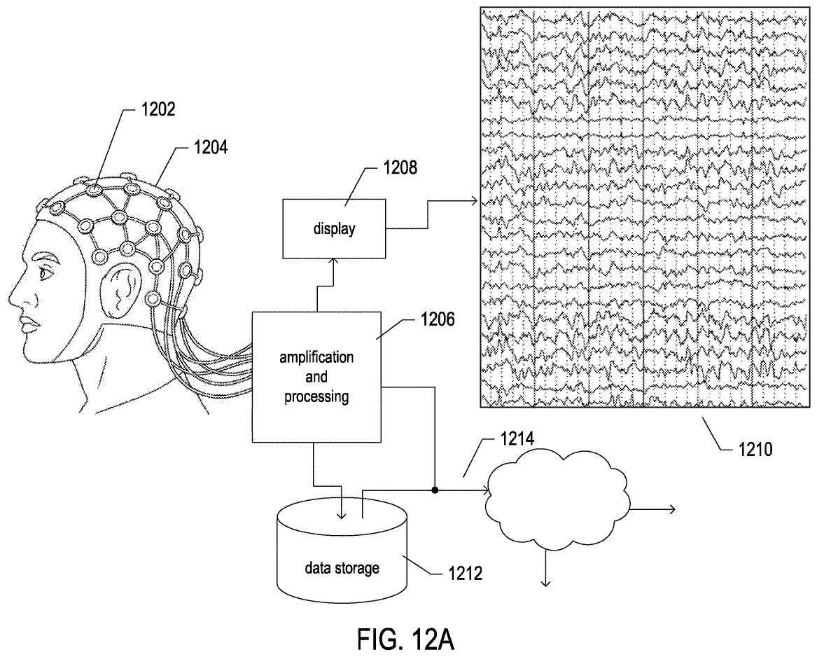

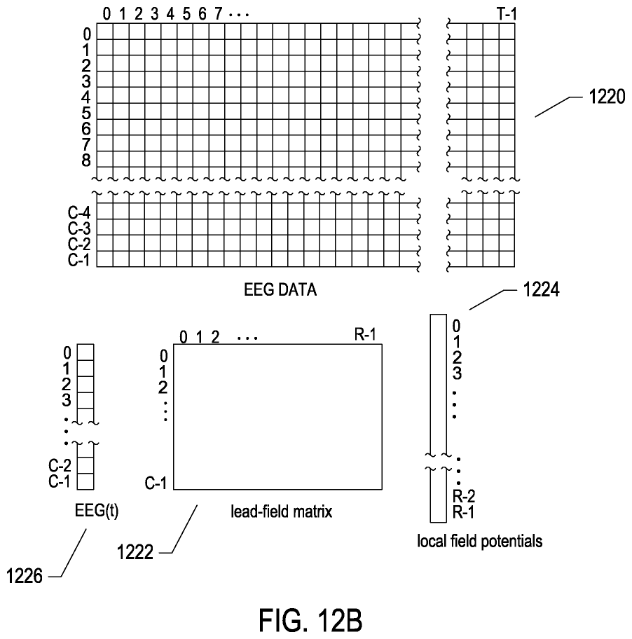

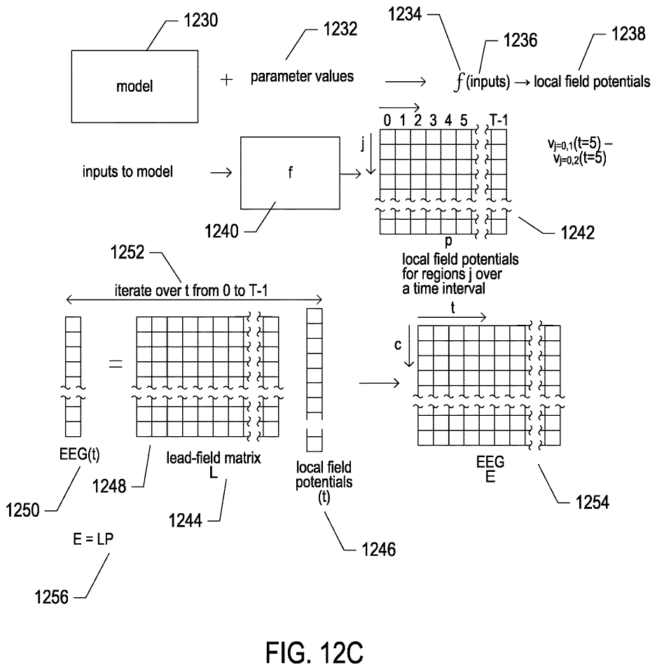

FIGS. 12A-C illustrate the electroencephalography (“EEG”) method for detecting and recording electrical activity within the brain. Voltage fluctuations within the cerebral cortex are sensed by EEG electrodes, such as EEG electrodes 1202, that are positioned at well-known locations on the scalp corresponding to underlying regions of the cerebral cortex. Each EEG electrode is connected to an input of a differential amplifier, with one amplifier for each pair of electrodes. The output of the amplifiers is processed to generate voltage/time curves for multiple channels, with the voltage/time curves indicating the voltage differences at successive time points between pairs of electrodes or between individual electrodes and a reference voltage. The voltage/time curves can then be displayed and/or digitally stored for computational processing and later display. In FIG. 12A, the voltage signals generated from electrodes attached to a skullcap 1204 are transmitted to an amplification and processing module 1206 which can output analog voltage/time curves to display equipment 1208 for printing 1210 and which can convert analog voltage signals to digital signals for display on a computer display or by other means and for digital storage in various different data-storage devices and appliances 1212. In addition, digital voltage/time curves can be transmitted 1214 to remote computers and other receiving devices for downstream display and processing.

As discussed above, the voltage fluctuations measured by the EEG method generally originate in the cerebral cortex due to synchronized depolarization and repolarization of apical dendrites emanating from pyramidal neurons. The voltage/time curves for each of the multiple EEG channels can exhibit various different frequencies of oscillation, spikes, and various different types of complex voltage-fluctuation patterns that can be correlated with various types of brain activities and pathologies.

As shown at the top of FIG. 12B, EEG data obtained from a patient over a time interval can be represented as a matrix 1220. The rows of the matrix each represent an EEG channel and are labeled with row indices 0 to C−1, where C is the number of channels output by the EEG method. The columns of the matrix represent points in time and are labeled with column indices 0 to T−1, where Tis the total number of time points in the time interval over which EEG data was collected. A different matrix 1222, referred to as the “lead-field matrix,” characterizes the responsiveness of each EEG electrode or electrode pair to every potential neural source within the brain. The rows of the lead-field matrix correspond to EEG channels, just as in the EEG data matrix 1220, and the columns of the lead-field matrix correspond to neural sources or brain regions and are labeled with column indices that range from 0 to R−1, where R is the total number of different brain regions that are considered. Multiplying a column vector 1224 containing the local field potentials of the various different R brain regions at a particular point in time by the lead-field matrix produces a column vector 1226 containing the EEG voltage signal at the particular point in time for each EEG channel.

FIG. 12C illustrates the use of the lead-field matrix to generate simulated EEG data. A computational model 1230 can be generated for simulating electrical activity in the brain. Several computational models are described in detail, below. When the computational model is initialized with parameter values 1232, it acts like a function 1234 that receives, as arguments, signal inputs 1236 and generates local field potentials for the various brain regions supported by the computational model 1238. Thus, given an initialized model 1240, inputs to the model results in the output, represented by matrix 1242, of local field potentials for each brain region j at each of T time periods. The rows of the local-field-potentials matrix P 1242 represent brain regions and the columns of matrix P represent time points. The matrix P is obtained by running the model for a time interval including T time points. As indicated in the sub-diagram 1244, multiplying a column vector of local field potentials for a particular point in time 1246 by the lead-field-matrix 1248 produces a column vector 1250 of EEG signal values. Iterating this process over each time point, as indicated by the labeled double-headed arrow 1252, produces an EEG data matrix 1254 equivalent to the EEG data matrix 1220 in FIG. 12B. A more concise, linear-algebra expression for this process 1256 indicates that multiplication of the matrix P from the left by the lead-field matrix L produces an EEG data matrix E.

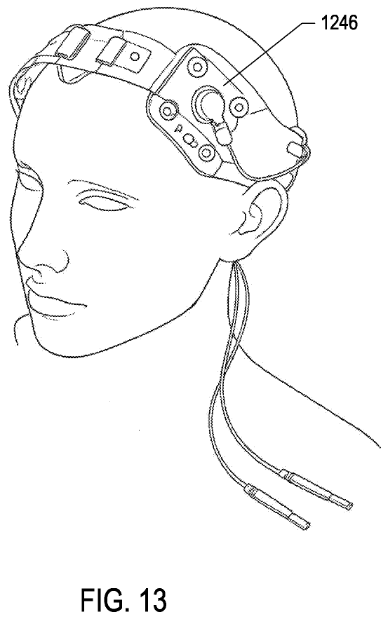

FIG. 13 illustrates application of direct-current transcranial stimulation (“tDCS”) to a patient. The tDCS method involves delivering a low-voltage direct current to regions of the cerebral cortex via electrodes 1302 applied to the scalp or skin. The current level, application, and patterns of direct-current application can be varied to produce different effects, in addition to selecting the particular brain region for stimulation. Direct-current stimulation may result in depolarization or hyperpolarization of neurons, increasing or decreasing neuronal excitability. The tDCS method has been shown to provide beneficial effects in treating depression and schizophrenia.

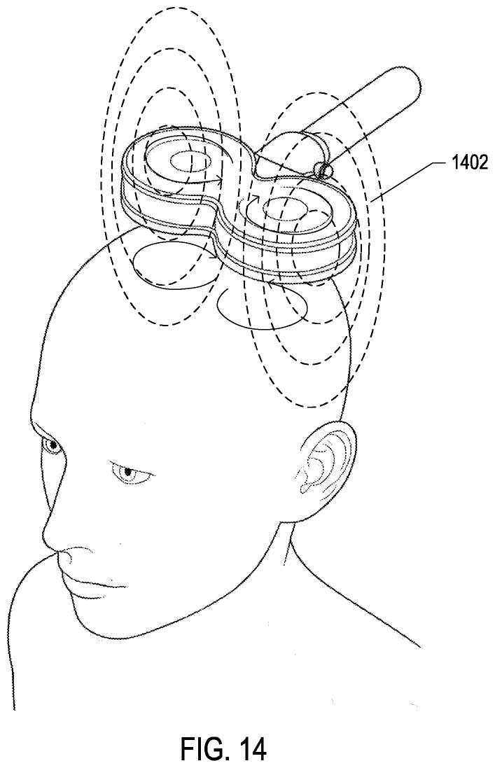

FIG. 14 illustrates application of transcranial magnetic stimulation (“TMS”) to a patient. In this method, electric pulses are input to a magnetic coil 1402 placed against the scalp. The magnetic coils produce a magnetic field that penetrates the skill and induces a secondary electric current in the underlying brain region. TMS has shown to be effective for treating depression, chronic pain, and obsessive-compulsive disorder in addition to various additional neurological and psychiatric conditions. Like application of tDCS, various parameters, such as the frequency, duration, and intensity of stimulation, can be varied to produce different effects of varying magnitude.

### An Overview of Convolution

FIGS. 15A-C illustrate the integral transform referred to as “convolution.” The convolution operation generates a third function (h*x)(t) from two functions h(t) and x(t), with the order of the two functions in the convolution operation irrelevant. In other words, the function (h*x)(t) is the same as the function (x*h)(t). Expression 1502 at the top of FIG. 15A defines the convolution operation. The variable T in the integration is a dummy variable and the variable t has a value in the domain of the functions h(t) and x(t). For example, the domain of the functions may be time and the functions produce a real-number value for each input time point. FIGS. 15A-B illustrate the convolution operation using a simple function x(t) shown in plot 1504 and the function h(t) shown in plot 1506. The convolution operation can be understood as a series of operations. The first operation is to reflect function h(t) about the t=0 axis 1508 to produce the reflected function h′(t) plotted in plot 1510. This reflection corresponds to the −τ portion of the argument t−τ to function h(in the integral on the right-hand side of expression 1502. Then, as illustrated in plot 1512, the reflected function h′(t) is translated, time point by time point, along the horizontal axis with respect to function x(t). While discrete translation steps are shown in plot 1512, the translation is continuous rather than stepwise. At each point t, the product of h(t −τ) and x(τ) is integrated over the entire domain, with the dummy variable T ranging from −∞ to ∞. For example, during the integration at time point t, as shown in plot 1514, the product h(t −τ) and x(τ) at time point τ is the product of the magnitudes of the vertical displacements 1516 and 1517. As shown in FIG. 15B, the value of the interval on the right hand side of expression 1502 is equal to the product of the areas under the curves of the reflected function h′(t) 1520 and x(t) 1522. Thus, in the plot of (h*x)(t) 1524, the value of the function 1526 at time point t 1528 is equal to the product of the areas underneath the reflected function h′(t) and function x(τ) in the overlap region of the two functions 1530. FIG. 15C illustrates the convolution of two different functions h(t) and x(t).

### An Overview of the Jansen-Rit Neural Mass Model

FIGS. 16A-C illustrate the Jansen-Rit neural mass model. This is a relatively simple computational model for modeling electrical activity in cortical units. As shown in FIG. 16A, a cortical unit 1602 refers to a volume of cerebral-cortex tissue, often associated with one or more functions. As discussed above, changing voltage potentials within and between such regions, detected by methodologies such as EEG, arise from the synchronized changing polarizations of apical dendrites emanating from pyramidal neurons. The computational model 1604 considers a population of pyramidal neurons 1606, a population of excitatory interneurons 1607, and a population of inhibitory interneurons 1608. The pyramidal neurons output excitatory signals to a first group of synapses 1610 interconnecting the pyramidal neurons with excitatory interneurons 1607 and to a second group of synapses 1611 interconnecting the pyramidal neurons with inhibitory interneurons 1608. The excitatory interneurons 1607 output excitatory signals to a third group of synapses 1612 interconnecting the excitatory interneurons with the pyramidal neurons 1606 and the inhibitory interneurons 1608 output excitatory signals to a fourth group of synapses 1613 that interconnect the inhibitory interneurons with the pyramidal neurons 1606. The pyramidal neurons additionally receive excitatory input 1614 from external cortical units and subcortical structures. The excitatory input, or external input, 1614 is represented by an average firing rate which can be random, deterministic, or a combination of random and deterministic signals that model electrical activity in interconnecting cortical units. The sum y of the inputs to the pyramidal neurons is both input to the pyramidal neurons and considered to be the output 1616 from the cortical unit. Note that, in FIG. 16A, excitatory signals are labeled with circled plus signs and inhibitory signals are labeled with circled minus signs. While this model is relatively simple, and abstracts many of the complexities of actual cortical units and brain tissue, it has been used to reliably model aggregate electrical activity in human brains.

FIG. 16B illustrates the Jansen-Rit model using a system-implementation diagram. The pyramidal neurons are represented by the contents within an area delimited by dashed line 1620, the excitatory interneurons are represented by the contents of the dashed-line rectangle 1622, and the inhibitory neurons are represented by the contents of dashed-lined rectangle 1624. The groups of synapses interconnecting the populations of neurons are represented by labeled ellipses 1626-1629. The inputs 1630-1634 to each population of neurons are average firing rates. These inputs are converted into average membrane potentials via an impulse-response function for excitatory signals 1635-1637 and an impulse-response function for inhibitory signals 1638. The output average membrane potential y generated from an input firing rate is the convolution product of the input firing rate and the impulse response function, which can also be represented by a differential equation. Output signals are generated by converting membrane potentials to firing rates via sigmoidal gain functions 1640-1642. A large group of cortical units can be interconnected via the inputs and outputs of the cortical unit and the computational model can be iterated over multiple time points within a time interval to generate the matrix of local field potentials P (1242 in FIG. 12C) that represents electrical activity with the brain and can be used to generate simulated EEG signals. Each region j is represented by one or more instances of the Jansen-Rit model illustrated in FIGS. 16A-B.

FIG. 16C provides the mathematical equations for one implementation of the Jansen-Rit model. Expression 1650 defines one implementation of the impulse-response function used to transform input firing rates to membrane potentials, where α and β are constants representing the maximal post-synaptic-potential amplitude and delays in synaptic transmission, respectively 1651. Expression 1652 restates the fact that the output membrane potential is the convolution of the input firing rate and the impulse-response function, as discussed above. The corresponding differential equation is shown as expression 1653. The sigmoidal gain function used to convert membrane potentials to firing rates is shown as expressions 1654. As indicated by expression 1655, the groups of synapses (1626-1629 in FIG. 16B) are represented in the mathematical model by constants corresponding to the number of synapses in each group, which is proportional to the strength of coupling between populations of neurons. The core mathematical expressions for the Jansen-Rit neural mass model are provided in expressions 1656. These are coupled differential equations that can be solved by various different types of methods, including matrix methods that can be used to determine eigenvalues and eigenvectors that are, in turn, used to produce expressions for the membrane potentials of the neuron populations at a time t resulting from the external input, given particular values for the various constant parameters in the mathematical expressions shown in FIG. 16C, examples of which are provided by expressions 1658. A simple example of solving differential equations is the determination of a function of a particle's position with respect to time, x=f(t) from second-order differential equations that represent the particle's acceleration in three coordinate-axis directions and from initial conditions, including the particle's initial position and initial velocity.

### An Overview of the Adaptive Moment Estimation (“ADAM”) and Bayesian Optimization Techniques

FIG. 17 illustrates an application of the ADAM optimization method. In the example shown in FIG. 17, the ADAM optimization method is used to find the parameters θ₁, θ₂, . . . , θₙ for a function ƒ( ) defined by polynomial expression 1702 at the top of FIG. 17. The function ƒ( ) can be thought of as representing a process that is carried out on inputs represented by the variables x₁, x₂, . . . , xₙ to produce a corresponding output value y. The process can be used, in a series of experiments at each of T time points, to determine the value produced by the process for a set of known input-variable values. The optimization problem addressed by the ADAM optimization method in this example is to use the experimental data that includes input values for the variables x₁, x₂, . . . , xₙ at each of T time points and the observed experimental result yₜ at each time point t to determine the values of the parameters θ₁, θ₂, . . . , θₙ so that function ƒ( ) accurately represents the process. In this illustration, a matrix D 1704 includes T rows, each row containing n input variable values x₁, x₂, . . . , xₙ for the experiment conducted at a particular time point t. The vector Y 1705 contains the observed experimental results at each of the time points. The notation “Dₜ” refers to a vector of input variable values for a particular time point or, in other words, the transpose of the row of the matrix D for that time point. Application of the ADAM optimization method is used to determine the values of the parameters θ₁, θ₂, . . . , θₙ using the observed experimental results contained in matrix D and vector Y.

In the illustrated application of the ADAM optimization method, the mean-square-error loss function 1706 is used. This function sums the squared differences between the values returned by the function ƒ( ) with a current set of parameter values and the observed experimental results. Expression 1708 defines the calculation of the partial differential of the loss function with respect to a particular parameter θᵢ. The gradient of the loss function is a vector of partial differentials for the n parameters, as indicated by expression 1710. This gradient can be squared by squaring each of its elements, as shown in expression 1712.

The ADAM optimization method is represented by control-flow diagram 1714. In step 1716, the method receives the function ƒ( ), the experimental-data matrix D, and the experimental-data vector Y, sets the function parameters to initial values, sets values of several ADAM optimization parameters, sets a first-moment vector m to the 0 vector and a second-moment vector v to the 0 vector, and finally sets the variable num_consecutive to 0. The ADAM optimization parameters include: (1) α, a step size or learning rate; (2) β₁, a decay rate for the first moment; (3) β₂, a decay rate for the second moment; and (4) ε, a small value to prevent division by 0 in the parameter-update step. Then, in the for-loop of steps 1718-1726, each dataset time point t is considered. In step 1719, local vector variable m_{new} is set to the sum of the product of β₁ and m and the product of β₁−1 and the gradient vector, local vector variable v_{new} is set to the sum of the product of β₂ and v and the product of β₂−1 and the squared gradient vector, local vector variable mc is set to m_{new} divided by 1−α′₁, local vector variable vc is set to v_{new} divided by β′₂, vector variable θ_{new} is set to vector variable θ minus the product of α and m_{c} divided by the square root of v_{c} plus ε, and the local variable change is set to the magnitude of θ_{new} minus θ. When the value stored in local variable change is less than a first threshold value, as determined in step 1720, variable num_consecutive is incremented, in step 1721, and, in step 1722, the method determines whether the value stored in num_consecutive is greater than a second threshold value. When the value stored in num_consecutive is greater than the second threshold value, the method terminates in step 1723, returning the parameter values stored in a vector variable θ_{new}. Otherwise, control flows to step 1725. The value stored in local variable change is greater than or equal to the first threshold, as determined in step 1720, local variable num_conservative is set to 0, in step 1724, after which control flows to step 1725. In step 1725, the method determines whether the currently considered time point t is less than T. If so, then in step 1726, the currently considered time point is advanced, vector variable θ is set to θ_{new}, vector variable m is set to m_{new}, and vector variable v is set to v_{new}, after which control flows back to step 1719 for a next iteration of the for-loop of steps 1718-1726. The ADAM optimization method is thus similar to a gradient-dissent method except that a portion of the previously computed gradient is added to the current gradient and the second-moment gradient is similarly updated for use in updating the parameters, as indicated by the expressions included in step 1719.

FIGS. 18A-F illustrate Bayesian optimization. FIG. 18A provides expressions that explain Baye's rule, which is the foundation for Bayesian regression and Bayesian optimization. The symbol “H” denotes an event 1802. An event is some type of sample point or occurrence that can be associated with a probability. For example, consider a process or function that can be somehow called or invoked with one or more inputs and that returns a response y, but the internal operation or analytic form of the process or function is unknown. A response y obtained from invoking the process can be considered to be an event. The numbers displayed by a pair of thrown dice is another example of an event. P(H) indicates the initial or currently understood or believed probability of the occurrence of event H 1803. This initial probability is referred to as the “prior.” The symbol “E” denotes some type of subsequent data or evidence that might change the initial belief in the probability of event H 1804. The expression “P(H|E)” denotes the conditional probability of the event H in view of the subsequent data or evidence E and is referred to as the “posterior” 1805. The expression “P(E|H)” denotes the conditional probability of observing the subsequent data or evidence E in view of the occurrence of event H, and is referred to as the “likelihood” 1806. The expression “P(E)” denotes the probability of observing the data or evidence E, regardless of the occurrence of event H, and is referred to as the “marginal likelihood” 1807. The probability of observing the data or evidence E is greater than 0 and the sum of the probabilities of all possible additional evidence is equal to 1. The probability of observing the data or evidence E is independent of the probability of H. A well-known equality in probability theory is:

$$P ( A \vert B ) P ( B ) = P ( AB ) .$$

The above expression indicates that the product of the conditional probability of A given B and the probability of B is equal to the probability of A and B. Using this well-known equality and the above-introduced posterior and likelihood, expressions 1808 illustrates the derivation of Baye's rule, given by expression 1809. This rule can be restated using the names assigned to the various probabilities in expressions 1803-1807 as shown in expression 1810. Since P(E) is independent from P(H) and is simply a constant normalizing factor for a given E, Baye's rule is often shown as a proportionality 1811. In other words, when thinking of P(E|H) and P(H) as probability density functions, their sum is also a probability density function, but one that is not normalized meaning that the area under the curve is not equal to 1. Dividing this normalized probability distribution by P(E) generates a normalized probability distribution.

FIG. 18B illustrates Bayesian linear regression. In the example shown in FIG. 18B, it is assumed that there is a function ƒ( ) that receives n inputs and returns the sum of each input multiplied by a corresponding parameter, as shown in expression 1814. The parameters are not known with certainty, but initial parameter values based on prior knowledge and belief or obtained by educated guessing are used by the Bayesian-linear-regression method. There are m experimental results that can be used to determine the values of the parameters. The experimental results include m input vectors x₁, x₂, . . . , xₘ 1815 that each contain n input values for function ƒ( ) and a vector of results y 1816 containing m results generated by function ƒ( ) from the m input vectors. The vector θ 1817 contains initial values for the n parameters of ƒ( ). Each call to the function can be modeled as shown in expression 1818, where the vector E represents normally distributed noise. Therefore, since the noise represents the only non-deterministic values in the model, the differences between the experimental results and the results produced by the function ƒ( ) with a given set of parameter values is also normally distributed, as shown by expression 1819. Thus, an expression for the probability density function for the results is given by expression 1820 based on the canonical expression for the probability density for a normally distributed random variable. The symbol “S” denotes the experimental data 1821. Using the above-described Bayes' rule, an expression for the conditional probability density for the parameters of the function given the experimental data is easily derived 1822, which is equivalent to the expression 1823. Bayesian linear regression thus provides a full probability density function for all of the parameters in aggregate or individually. From these expressions, a posterior predictive probability distribution 1824 can be derived, which provides a predicted probability density function for the result of a new data vector. An alternative method for linear regression is the method of least squares. When there is sufficient experimental data of sufficient quality, this method can be used to generate specific values for the parameters along with confidence intervals. By contrast, the Bayesian linear regression method uses not only the experimental data but also any initial beliefs with regard to the parameter values and returns a full probability density function for each parameter value.

FIG. 18C illustrates radial functions, radial kernels, radial basis functions, and function approximation using radial basis functions. A radial function takes a positive real number as an argument and returns a real number value 1826. A radial kernel takes a vector input x as input and returns the distance between the input vector x and a position vector c, as indicated by expressions 1827. A radial function φ and the radial kernels derived from the radial function φ_{c} constitute a set of radial basis functions when the radial kernels are linearly independent and form a basis for a Haar Space 1828. FIG. 18C shows one example of a set of radial basis functions 1829 referred to as inverse quadratic radial basis functions. Finally, radial basis functions can be used to approximate functions as the sum of weighted radial kernels, as indicated by expression 1830.

FIG. 18D introduces Bayesian optimization. The problem addressed by Bayesian optimization is the identification of a maximum or minimum value of a function for which particular values at particular input points can be determined at relatively high computational cost, but for which there is no closed-form expression 1832. Such functions are referred to as “black-box functions.” Note that, in general, the points are points within high-dimensional spaces and thus represented by vectors while the values generated by the black-box function are real scalar values. Bayesian optimization solves this problem, as indicated in text 1833, by modeling the function as a sampling of a Gaussian process. This involves iteratively finding the value of the function at a next point and adding the next point and associated value to a set of points and associated values representing a collection of random variables with a particular type of distribution. The distribution of the current collection of random variables in a particular iteration is used to predict the function value at arbitrary points along with a confidence interval for the prediction. Surrogate functions for the black-box function sampled from the Gaussian distribution defined by the mean function and covariance function are then used to select a next point at which to compute a corresponding value using the black-box function in order to begin a next iteration. A control-flow diagram for Bayesian optimization is provided in FIG. 18E and discussed below. When m values of the function have been computed in m iterations of the Bayesian-optimization process for m points, the vector of collected points and computed values 1834 represents a set of random variables that is distributed as a multivariate Gaussian defined by a mean function 1835 and a covariance function 1836. The elements of the mean function include the expected values of the random variables 1837 and the covariance function assigns, to each pair of points so far collected, the covariance between the computed values of these points 1838. In many implementations, one of the above-discussed inverse quadratic radial basis functions 1839 is used to generate the covariances included in the covariance function 1840.

Thus, once one or more values for one or more points have been computed using the black-box function, the mean function and covariance function based on the collected values can be computed as indicated in expressions 1842 and 1843 in FIG. 18D. This allows for sampling surrogate functions 1844 from the normal distribution characterized by the mean function and covariance function. The surrogate functions are then used to select a best next point for computing a next value using the black-box function. The next point is selected as a point that provides the greatest expected improvement in the approximation of the black-box function by the surrogate functions obtained by sampling the multivariate distribution for a next surrogate function, as indicated in expression 1845 at the bottom of FIG. 18D. In the example illustrated in FIGS. 18D-F, a minimum value of the black-box function is sought. The variable min value is the smallest value so far computed by the black-box function and vₓ is a value returned by a sampled surrogate function for point x. The point x for which the expected improvement is greatest is selected as the next point for computing a value using the black-box function, and finding point x involves use of analytical expressions derived from expression 1845 at the bottom of FIG. 18D. Such a point is generally associated with a value returned by a surrogate function that is less than the minimum value so far returned by the black-box function and that is in a region of relatively high uncertainty as determined by the confidence intervals associated with the points in the domain of the surrogate functions.

FIG. 18E provides a control-flow diagram for a routine that implements Bayesian optimization. In step 1850, the routine receives a black-box function ƒ( ) and any additional information about ƒ( ) and initial beliefs with regard to the parameter values for the surrogate functions sampled from the Gaussian process or best points at which to begin the optimization process. In step 1851, the routine selects an initial evaluation point p and initializes the set of points Ps. In step 1852, the routine uses the black-box function to compute a value v for point p, associates the value v with p in set Ps, sets local variable min to p, v, and generates the posterior distribution parameters μ(p) and σ²(p) according to expressions 1842-1843 in FIG. 18D. Following this initialization phase, the routine begins iterating the loop of steps 1854-1862. In step 1854, the routine selects a next point p for evaluation by the black-box function, as discussed above with reference to expression 1845 in FIG. 18D. In step 1855, the routine determines whether or not the optimization has converged. There are many different possible tests for convergence. Convergence may be indicated by less than a threshold amount of improvement observed for the next selected point, as discussed above with reference to expression 1845 and FIG. 18D. Convergence may also be indicated when the uncertainty across the domain of the black-box function falls below some minimum threshold. Yet another test for convergence is whether the next computed minimal value is insufficiently less than the lowest minimal value so far observed. Iterations may also be discontinued when some maximum number of iterations has been reached. If optimization has converged, then the minimal value and point at which the minimum value was observed are returned in step 1856. Otherwise, in step 1857, the routine employs the black-box function to compute a value for the next point p. When the value v is less than the value stored in local variable min, as determined in step 1858, local variable min is set top, v in step 1859. In step 1860, p in association with v is added to the set Ps and new posterior-distribution parameters are computed for the set, as discussed above with reference to expressions 1842-1843 in FIG. 18D. Again, in step 1861, convergence is tested. If convergence is detected, the routine returns the point and minimum value stored in local variable min. Otherwise, control returns to step 1854 for another iteration of the loop of steps 1854-1862.

FIG. 18F shows a sequence of steps for a hypothetical Bayesian optimization. The steps are illustrated by plots, including plot 1870 for the first step in which a first point is evaluated using the black-box function. In the first step, the evaluated points and the confidence range across the domain are plotted with respect to a pair of axes 1871 and the expected improvement across the domain 1872 is plotted below in alignment with the horizontal coordinate axis. The plots are 2-dimensional, for ease of illustration, with points falling within a single dimension corresponding to the horizontal axis and the values computed by the black-box function for the point plotted with respect to the vertical axis. As discussed above, however, Bayesian optimization is generally carried out on points in higher-dimensional spaces represented by vectors. In the first step, the black-box function was evaluated at an initial point 1873. Dashed curve 1874 represents the upper values and dashed curve 1875 represents the lower values of the confidence intervals that vertically span each point in the domain. As the value of only one point has been computed using the black-box function, the confidence intervals collapse to that single point/value. Note also that the expected improvement is 0 at that point 1876 but rises to a large value on either side of the point. In step 1877, a second point 1878 has been evaluated using the black-box function. Again, the confidence intervals collapse at that point/value. Note that the point was selected based on its distance from the first point as well as the wide confidence interval at that point corresponding to the vertical separation of the confidence curves at the point in plot 1870. Each successive step 1879-1886 involves selection and evaluation of an additional point using the black-box function. Eventually, the evaluated points and confidence intervals converge to an accurate approximation of the black-box function.

### An Overview of the Kuramoto Synchronization Model

FIGS. 19A-B illustrate the Kuramoto model for synchronization among multiple coupled oscillators. FIG. 19A illustrates oscillators and phases. In FIG. 19A, two oscillators are considered, both pendulums. The positions of the arm of the first oscillator at each of successive time points are shown in a top row 1902 of FIG. 19A and the positions of the arm of the second oscillator at each of successive time points are shown in a bottom row 1904 of FIG. 19A. The positions of the arms of the two oscillators are plotted in a central plot 1906 as two curves, a first curve 1908 for the first oscillator shown in the top row 1902 and a second curve 1910 for the second oscillator shown in the bottom row 1904. The horizontal axis of the plot 1912 represents time and the vertical axis 1914 of the plot represents the amplitude of the oscillator. At time point 1916, the arm 1917 of the first oscillator 1918 has swung all the way to the left and the first oscillator has an amplitude of −A. At time point 1919, the arm 1920 of the first oscillator is vertical and the first oscillator has an amplitude of 0. At time point 1921, the arm 1922 of the first oscillator has swung all the way to the right and the first oscillator has an amplitude of A. The amplitude is thus the signed horizontal distance between the weight of the pendulum and the vertical pendulum support. In FIG. 19A, relative times are assigned to each of the two oscillators. For the first oscillator, relative time −π/2 (1923) corresponds to time point 1916, when the oscillator arm 1917 has swung all the way to the left, relative time 0 (1924) corresponds to time point 1919, when the oscillator arm 1920 is vertical, and relative time π/2 (1925) corresponds to time point 1921, when the oscillator arm 1922 has moved all the way to the right. Thus, the portion of the curve 1908 for the first oscillator between time points 1916 and 1921 represents the change in amplitude with respect to time for the first oscillator from when the pendulum is fully extended to the left to when the pendulum arm is fully extended to the right. The portion of the curve 1908 for the first oscillator between time point 1919 and time point 1926 represents an entire cycle for the first oscillator, during which the pendulum arm swings all the way to the right from the vertical position, reverses direction and swings all the way to the left, and then returns to the vertical position. In the relative time for the first oscillator, each cycle begins at relative time 2nπ, where n is an integer that ranges from −∞ to +∞. Curve 1908 corresponds to the function y=A sin(t), where t is the relative time for the oscillator. Knowing the length of the time interval in seconds or some other unit of time between time point 1919 and time period 1926, L, allows for the determination of the frequency of the oscillation. The frequency ω is equal to 1/L or, in other words, one cycle per L units time. Thus, the curve can also be expressed using real-time as y=A sin(2πt/L+φ), where φ is the phase at time t=0. The second oscillator 1928 oscillates with the same frequency as the first oscillator, in the example shown in FIG. 19A, but is out of phase with respect to the first oscillator, meaning that the relative time for the second oscillator is different than the relative times of the first oscillator. For example, at time point 1921, the pendulum arm of the second oscillator 1929 is vertical and the relative time for the second oscillator is 0 1930. The relative time can also be described as a phase angle. The pendulum arm is vertical for phase angles 0 and π, is all the way to the right for phase angle π/2, and is all the way to the left for phase angle −π/2 or 3π/2. Phase angles range from 0 to 2π. In the example shown in FIG. 19A, as indicated in expression 1932, the phase angle for the second oscillator θ₂ is equal to the sum of the phase angle for the first oscillator θ₂ and π/2. The second oscillator can be thought of as lagging behind the first oscillator by a phase angle of π/2. Equivalently, as shown in expression 1934, the second oscillator can be thought of as lagging behind the first oscillator by a phase angle of 3π/2. Note that a given phase angle θ is equivalent to phase angles of θ+2πn, where n is an integer. In the example of FIG. 19A, both oscillators have the same amplitude range and frequency, or number of cycles per second. In a more general case, each oscillator would have a different amplitude and a different frequency from the other oscillator. The curves for the two oscillators, in that case, would not have the same peak heights and appear to be translated by a fixed distance from one another across time, but would instead exhibit a more complex relationship. The correspondence of the phase angle for an oscillator and the position of the moving part of the oscillator with respect to a reference point is arbitrary, but is often selected based on some natural correspondence between the configuration of the oscillator and a logical phase of 0 or 2π.

FIG. 19B illustrates the Kuramoto model. In a first row 1940 at the top of FIG. 19B, phase indications for five different oscillators are shown. Each oscillator is identified by an index i selected from the set {1, 2, 3, 4, 5}. Each of the oscillators has its own intrinsic natural frequency ωᵢ and phase θᵢ. However, when the oscillators are mechanically coupled, it is often observed that the oscillators, over time, fully or partially synchronize, with a partial synchronization shown in the phase indications in 1942 in which 4 of the 5 oscillators have nearly the same phases and frequencies.

The Kuramoto model is a set of coupled differential equations, one for each oscillator, as indicated by expression 1944 in FIG. 19B. The term following the summation sign tends to force the phase of oscillators toward a common phase when the coupling constant is relatively strong. When the frequency term has the same value for all of the oscillators, their change in phase with time tends toward a constant value. As indicated in text 1946, depending on the coupling constant and other parameters, the set of oscillators can completely synchronize, partially synchronize, form sets of synchronized clusters, form sets of synchronized clusters along with one or more unsynchronized oscillators, or fail to synchronize altogether. Synchronization is related to conservation of energy, and, when multiple coupled oscillators synchronize, energy dissipation is generally minimized.

### An Overview of Neural Networks

FIG. 20 illustrates fundamental components of a feed-forward neural network. Expressions 2002 mathematically represent ideal operation of a neural network as a function ƒ(x). The function receives an input vector x and outputs a corresponding output vector y 1103. For example, an input vector may be a digital image represented by a 2-dimensional array of pixel values in an electronic document or may be an ordered set of numeric or alphanumeric values. Similarly, the output vector may be, for example, an altered digital image, an ordered set of one or more numeric or alphanumeric values, an electronic document, or one or more numeric values. The initial expression of expressions 2002 represents the ideal operation of the neural network. In other words, the output vector y represents the ideal, or desired, output for corresponding input vector x. However, in actual operation, a physically implemented neural network \hat{ƒ}(x), as represented by the second expression of expressions 2002, returns a physically generated output vector ŷ that may differ from the ideal or desired output vector y. An output vector produced by the physically implemented neural network is associated with an error or loss value. A common error or loss value is the square of the distance between the two points represented by the ideal output vector y and the output vector produced by the neural network ŷ. The distance between the two points represented by the ideal output vector and the output vector produced by the neural network, with optional scaling, may also be used as the error or loss. A neural network is trained using a training dataset comprising input-vector/ideal-output-vector pairs, generally obtained by human or human-assisted assignment of ideal-output vectors to selected input vectors. The ideal-output vectors in the training dataset are often referred to as “labels.” During training, the error associated with each output vector, produced by the neural network in response to input to the neural network of a training-dataset input vector, is used to adjust internal weights within the neural network in order to minimize the error or loss. Thus, the accuracy and reliability of a trained neural network is highly dependent on the accuracy and completeness of the training dataset.

As shown in the middle portion 2006 of FIG. 20, a feed-forward neural network generally consists of layers of nodes, including an input layer 2008, an output layer 2010, and one or more hidden layers 2012. These layers can be numerically labeled 1, 2, 3, . . . , L−1, L, as shown in FIG. 20. In general, the input layer contains a node for each element of the input vector and the output layer contains one node for each element of the output vector. The input layer and/or output layer may each have one or more nodes. In the following discussion, the nodes of a first level with a numeric label lower in value than that of a second layer are referred to as being higher-level nodes with respect to the nodes of the second layer. The input-layer nodes are thus the highest-level nodes. The nodes are interconnected to form a graph, as indicated by line segments, such as line segment 2014.

The lower portion of FIG. 20 (2020 in FIG. 20) illustrates a feed-forward neural-network node. The neural-network node 2022 receives inputs 2024-2027 from one or more next-higher-level nodes and generates an output 2028 that is distributed to one or more next-lower-level nodes 2030. The inputs and outputs are referred to as “activations,” represented by superscripted-and-subscripted symbols “a” in FIG. 20, such as the activation symbol 2024. An input component 2036 within a node collects the input activations and generates a weighted sum of these input activations to which a weighted internal activation a₀ is added. An activation component 2038 within the node is represented by a function g( ), referred to as an “activation function,” that is used in an output component 2040 of the node to generate the output activation of the node based on the input collected by the input component 2036. The neural-network node 2022 represents a generic hidden-layer node. Input-layer nodes lack the input component 2036 and each receive a single input value representing an element of an input vector. Output-component nodes output a single value representing an element of the output vector. The values of the weights used to generate the cumulative input by the input component 2036 are determined by training, as previously mentioned. In general, the input, outputs, and activation function are predetermined and constant, although, in certain types of neural networks, these may also be at least partly adjustable parameters. In FIG. 20, three different possible activation functions are indicated by expressions 2042-2044. The first expression is a binary activation function and the third expression represents a sigmoidal relationship between input and output that is commonly used in neural networks and other types of machine-learning systems, both functions producing an activation in the range [0, 1]. The second function is also sigmoidal, but produces an activation in the range [−1, 1].

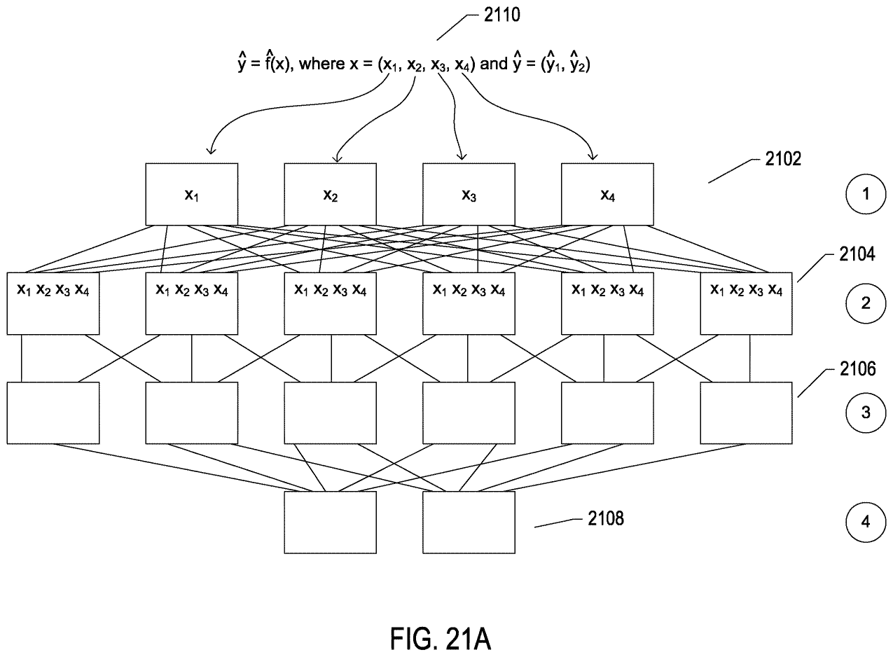

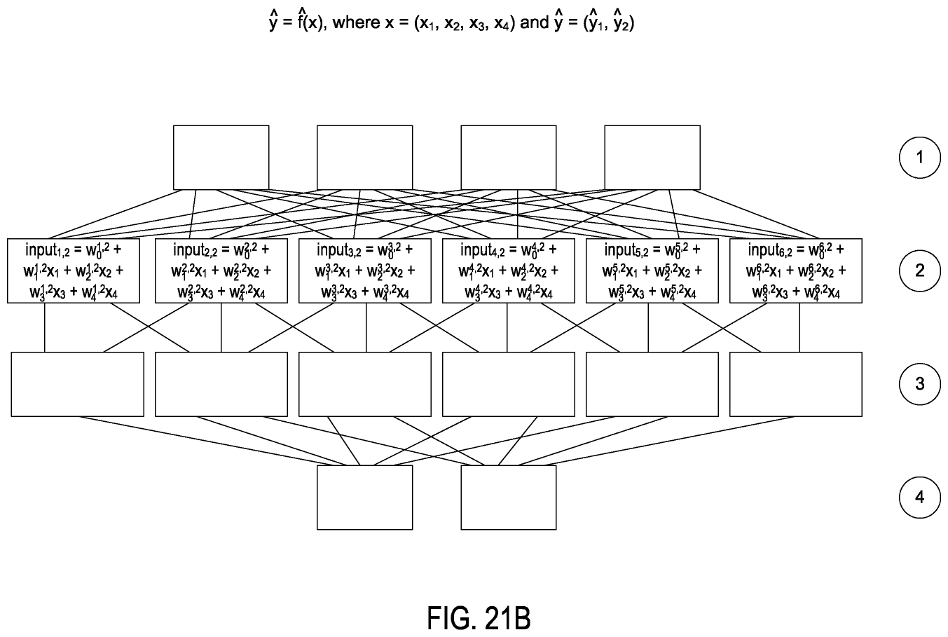

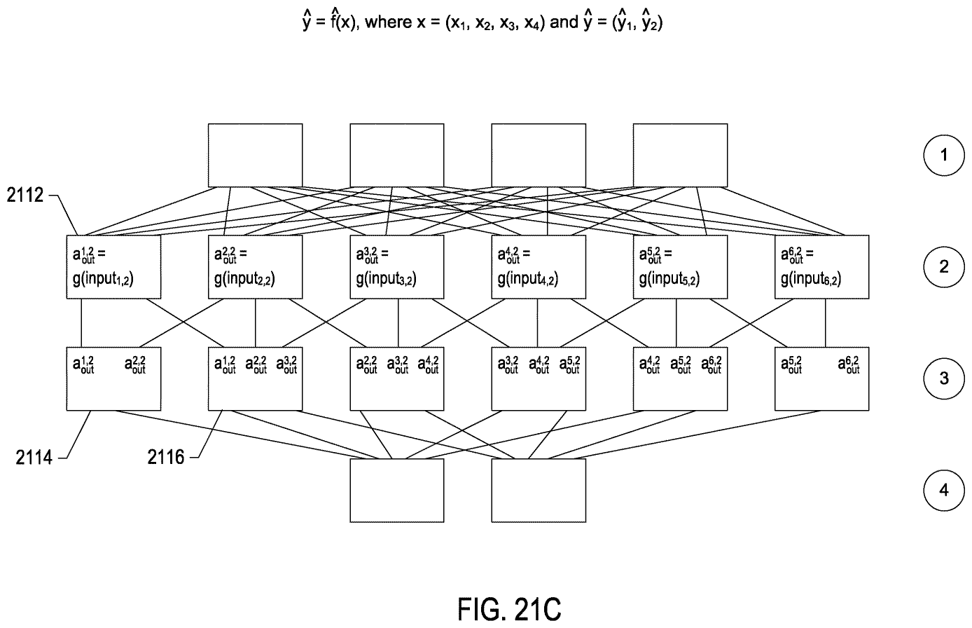

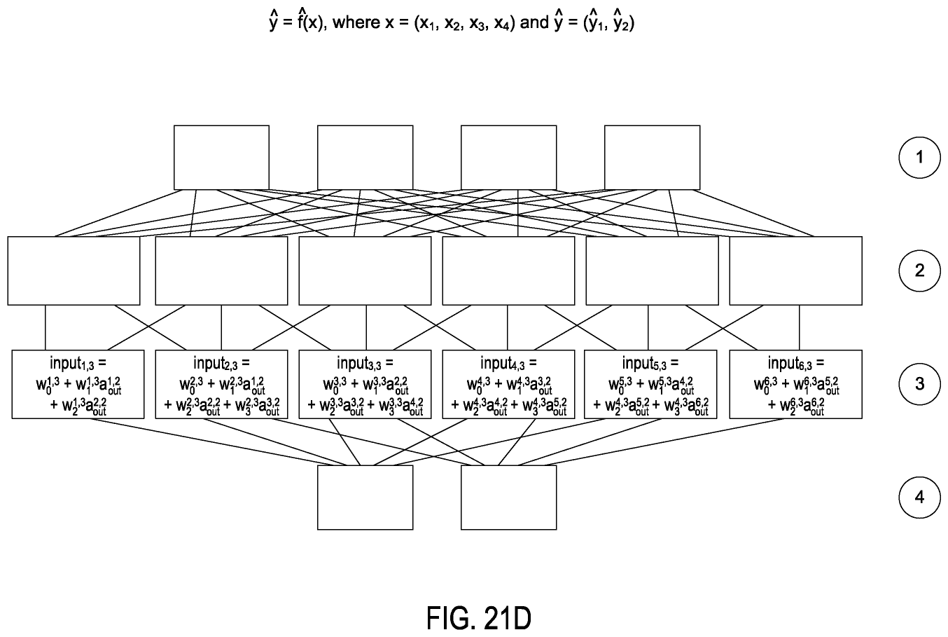

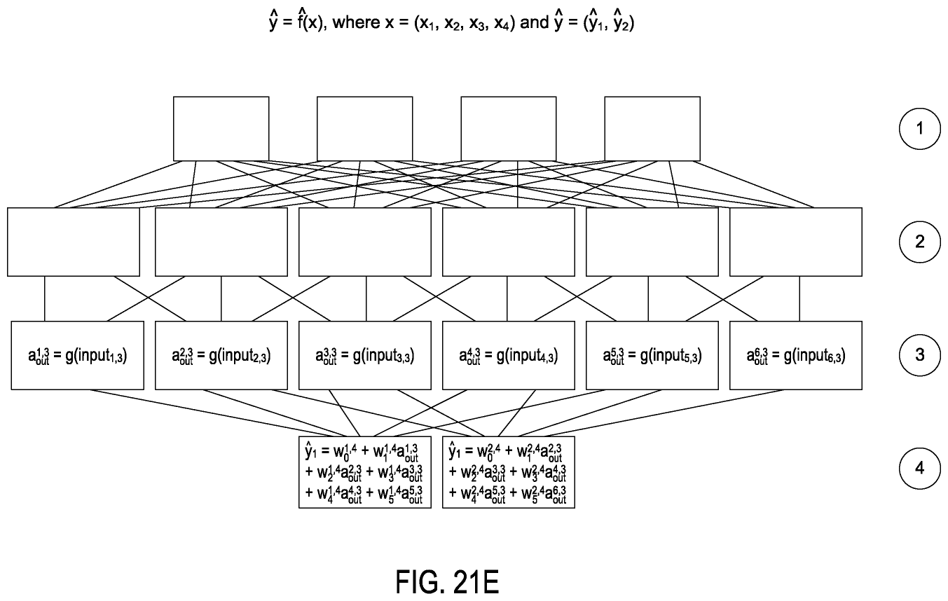

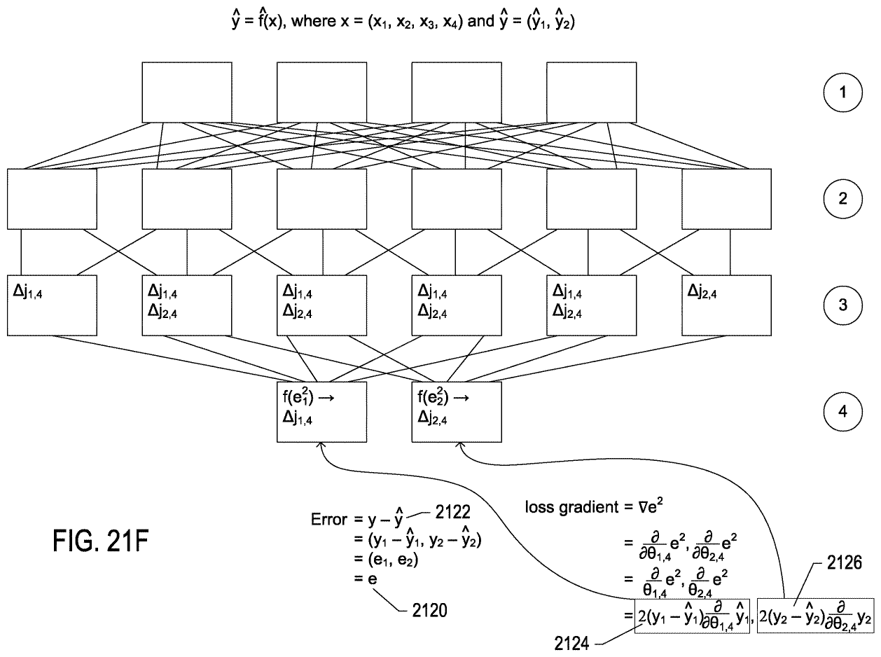

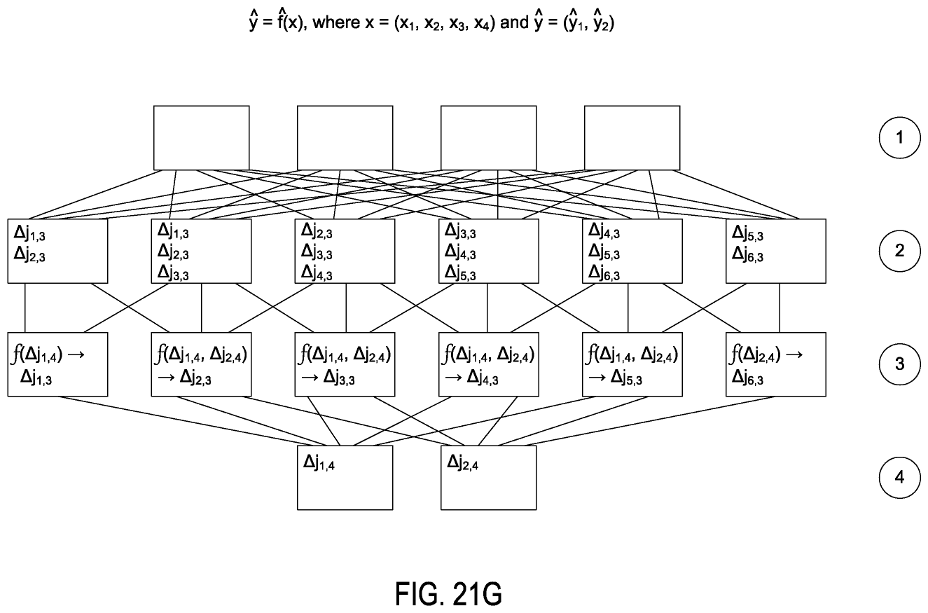

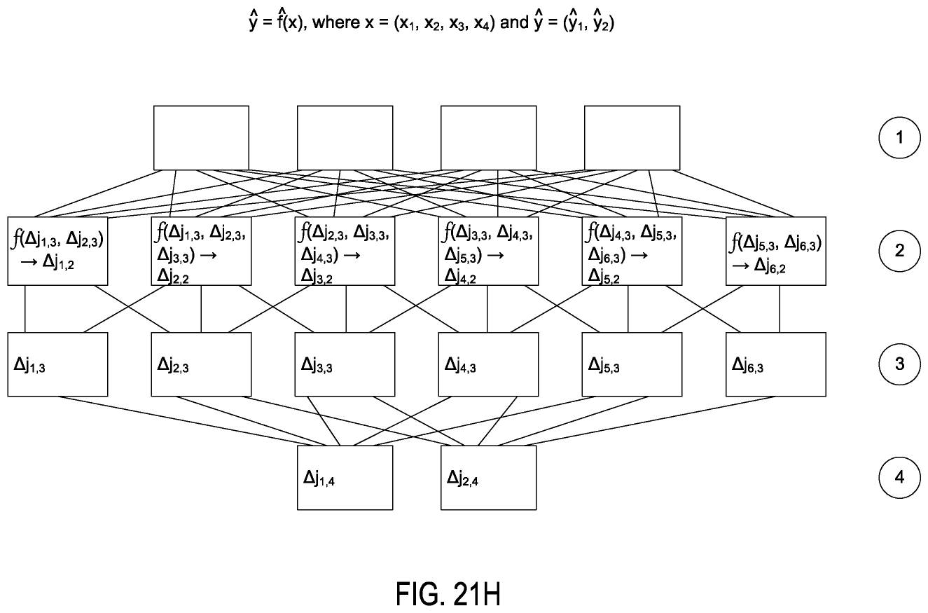

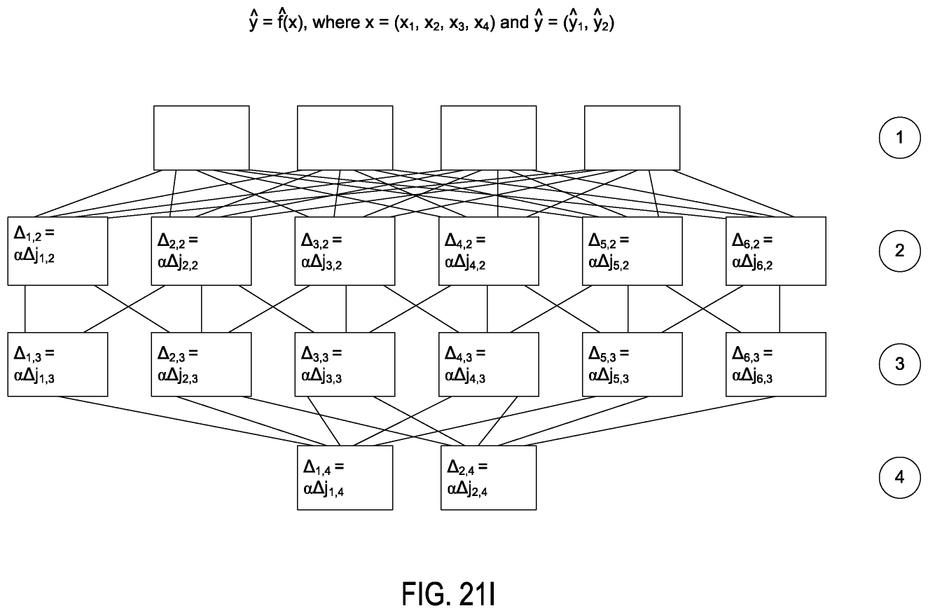

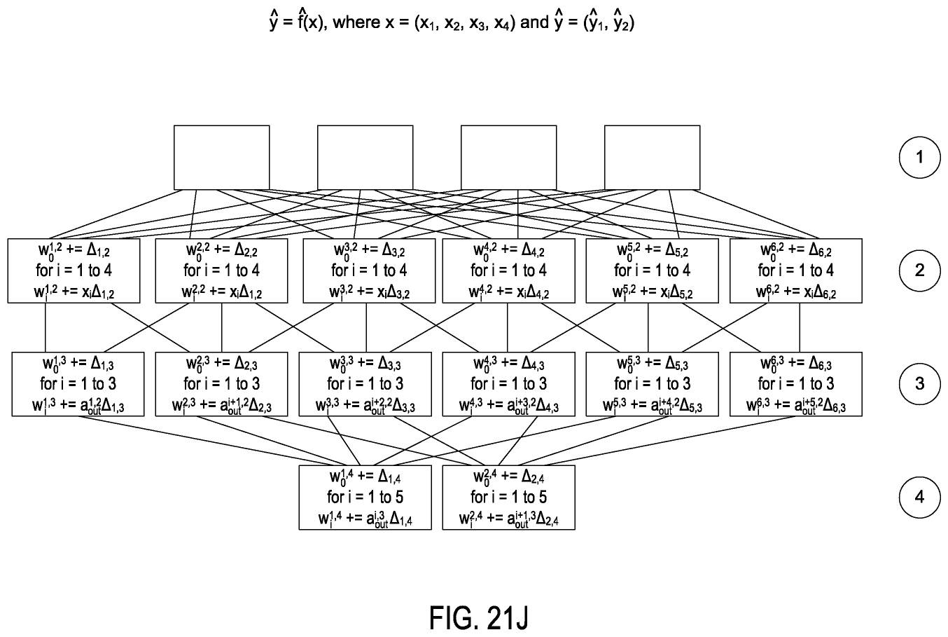

FIGS. 21A-J illustrate operation of a very small, example neural network. The example neural network has four input nodes in a first layer 2102, six nodes in a first hidden layer 2104 six nodes in a second hidden layer 2106, and two output nodes 2108. As shown in FIG. 21A, the four elements of the input vector x 2110 are each input to one of the four input nodes which then output these input values to the nodes of the first-hidden layer to which they are connected. In the example neural network, each input node is connected to all of the nodes in the first hidden layer. As a result, each node in the first hidden layer has received the four input-vector elements, as indicated in FIG. 21A. As shown in FIG. 21B, each of the first-hidden-layer nodes computes a weighted-sum input according to the expression contained in the input components (2036 in FIG. 20) of the first hidden-layer nodes. Note that, although each first-hidden-layer node receives the same four input-vector elements, the weighted-sum input computed by each first-hidden-layer node is generally different from the weighted-sum inputs computed by the other first-hidden-layer nodes, since each first-hidden-layer node generally uses a set of weights unique to the first-hidden-layer node. As shown in FIG. 21C, the activation component (2038 in FIG. 20) of each of the first-hidden-layer nodes next computes an activation and then outputs the computed activation to each of the second-hidden-layer nodes to which the first-hidden-layer node is connected. Thus, for example, the first-hidden-layer node 2112 computes activation aₒᵤₜ^{1,2} using the activation function and outputs this activation to second-hidden-layer nodes 2114 and 2116. As shown in FIG. 21D, the input components (2036 in FIG. 20) of the second-hidden-layer nodes compute weighted-sum inputs from the activations received from the first-hidden-layer nodes to which they are connected and then, as shown in FIG. 21E, compute activations from the weighted-sum inputs and output the activations to the output-layer nodes to which they are connected. The output-layer nodes compute weighted sums of the inputs and then output those weighted sums as elements of the output vector.

FIG. 21F illustrates backpropagation of an error computed for an output vector. Backpropagation of a loss in the reverse direction through the neural network results in a change in some or all of the neural-network-node weights and is the mechanism by which a neural network is trained. The error vector ŷ 2120 is computed as the difference between the desired output vector y and the output vector ŷ (2122 in FIG. 21F) produced by the neural network in response to input of the vector x. The output-layer nodes each receive a squared element of the error vector and compute a component of a gradient of the squared length of the error vector with respect to the parameters θ of the neural-network, which are the weights. Thus, in the current example, the squared length of the error vector e is equal to

$${\vert e \vert}^{2} or {e}_{1}^{2} + {e}_{2}^{2} ,$$

and the loss gradient is equal to:

$${∇}_{θ} ( {e}_{1}^{2} + {e}_{2}^{2} ) = \frac{\partial }{\partial θ} {e}_{1}^{2} , \frac{\partial }{\partial θ} {e}_{2}^{2} .$$

Since each output-layer neural-network node represents one dimension of the multi-dimensional output, each output-layer neural-network node receives one term of the squared distance of the error vector and computes the partial differential of that term with respect to the parameters, or weights, of the output-layer neural-network node. Thus, the first output-layer neural-network node receives

$${e}_{1}^{2}$$

and computes

$$\frac{\partial }{\partial {θ}_{1 , 4}} {e}_{1}^{2} ,$$

where the subscript 1,4 indicates parameters for the first node of the fourth, or output, layer. The output-layer neural-network nodes then compute this partial derivative, as indicated by expressions 2124 and 2126 in FIG. 21F. The computations are discussed later. However, to follow the backpropagation diagrammatically, each node of the output layer receives a term of the squared length of the error vector which is input to a function that returns a weight adjustment Δⱼ. As shown in FIG. 21F, the weight adjustment computed by each of the output nodes is back propagated upward to the second-hidden-layer nodes to which the output node is connected. Next, as shown in FIG. 21G, each of the second-hidden-layer nodes computes a weight adjustment Δⱼ from the weight adjustments received from the output-layer nodes and propagates the computed weight adjustments upward in the neural network to the first-hidden-layer nodes to which the second-hidden-layer node is connected. Finally, as shown in FIG. 21H, the first-hidden-layer nodes computes weight adjustments based on the weight adjustments received from the second-hidden-layer nodes. These weight adjustments are not, however, back propagated further upward in the neural network since the input-layer nodes do not compute weighted sums of input activations, instead each receiving only a single element of the input vector x.

In a next logical step, shown in FIG. 21I, the computed weight adjustments are multiplied by a learning constant α to produce final weight adjustments Δ for each node in the neural network. In general, each final weight adjustment is specific and unique for each neural-network node, since each weight adjustment is computed based on a node's weights and the weights of lower-level nodes connected to a node via a path in the neural network. The logical step shown in FIG. 21I is not, in practice, a separate discrete step since the final weight adjustments can be computed immediately following computation of the initial weight adjustment by each node. Similarly, as shown in FIG. 21J, in a final logical step, each node adjusts its weights using the computed final weight adjustment for the node. Again, this final logical step is, in practice, not a discrete separate step since a node can adjust its weights as soon as the final weight adjustment for the node is computed. It should be noted that the weight adjustment made by each node involves both the final weight adjustment computed by the node as well as the inputs received by the node during computation of the output vector ŷ from which the error vector e was computed, as discussed above with reference to FIG. 21F. The weight adjustment carried out by each node shift the weights in each node toward producing an output that, together with the outputs produced by all the other nodes following weight adjustment, results in decreasing the distance between the desired output vector y and the output vector ŷ that would now be produced by the neural network in response to receiving the input vector x. In many neural-network implementations, it is possible to make batched adjustments to the neural-network weights based on multiple output vectors produced from multiple inputs, as discussed further below.

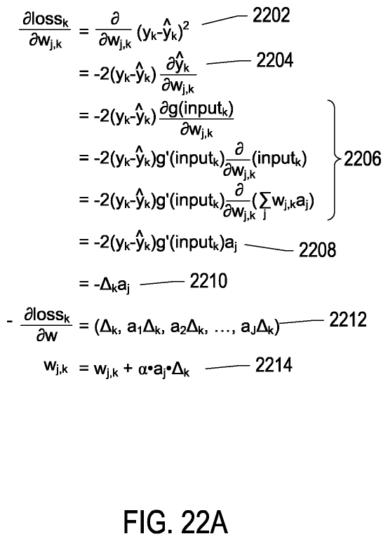

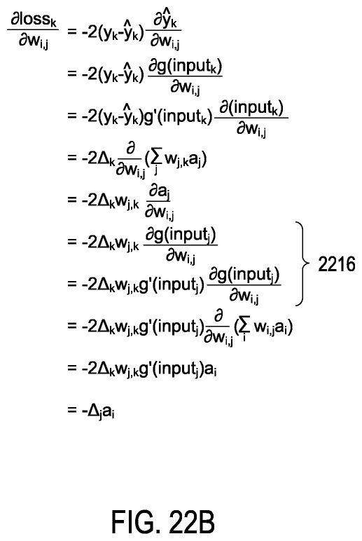

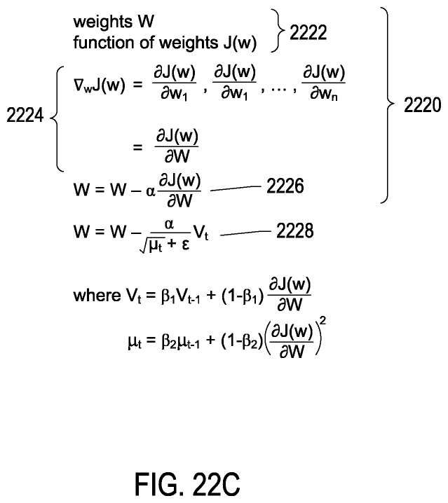

FIGS. 22A-C show details of the computation of weight adjustments made by neural-network nodes during backpropagation of error vectors into neural networks. The expression 2202 in FIG. 22A represents the partial differential of the loss, or k^{th} component of the squared length of the error vector eₖ², computed by the k^{th} output-layer neural-network node with respect to the J+1 weights applied to the formal 0^{th} input a₀ and inputs a₁-a_{J} received from higher-level nodes. Application of the chain rule for partial differentiation produces expression 2204. Substitution of the activation function for ŷₖ in the second application of the chain rule produces expressions 2206. The partial differential of the sum of weighted activations with respect to the weight for activation j is simply activation j, aⱼ, generating expression 2208. The initial factors in expression 2208 are replaced by −Δₖ to produce a final expression for the partial differential of the k^{th} component of the loss with respect to the j^{th} weight, 2210. The negative gradient of the weight adjustments is used in backpropagation in order to minimize the loss, as indicated by expression 2212. Thus, the j^{th} weight for the k^{th} output-layer neural-network node is adjusted according to expression 2214, where α is a learning-rate constant in the range [0,1].

FIG. 22B illustrates computation of the weight adjustment for the kth component of the error vector in a final-hidden-layer neural-network node. This computation is similar to that discussed above with reference to FIG. 22A, but includes an additional application of the chain rule for partial differentiation in expressions 2216 in order to obtain an expression for the partial differential with respect to a second-hidden-layer-node weight that includes an output-layer-node weight adjustment.

FIG. 22C illustrates one commonly used improvement over the above-described weight-adjustment computations. The above-described weight-adjustment computations are summarized in expressions 2220. There is a set of weights W and a function of the weights J(W), as indicated by expressions 2222. The backpropagation of errors through the neural network is based on the gradient, with respect to the weights, of the function J(W), as indicated by expressions 2224. The weight adjustment is represented by expression 2226, in which a learning constant times the gradient of the function J(W) is subtracted from the weights to generate the new, adjusted weights. In the improvement illustrated in FIG. 22C, expression 2226 is modified to produce expression 2228 for the weight adjustment. In the improved weight adjustment, the learning constant α is divided by the sum of a weighted average of adjustments and a very small additional term P and the gradient is replaced by the factor Vₜ, where t represents time or, equivalently, the current weight adjustment in a series of weight adjustments. The factor Vₜ is a combination of the factor for the preceding time point or weight adjustment Vₜ₋₁ and the gradient computed for the current time point or weight adjustment. This factor is intended to add momentum to the gradient descent in order to avoid premature completion of the gradient-descent process at a local minimum. Division of the learning constant α by the weighted average of adjustments adjusts the learning rate over the course of the gradient descent so that the gradient descent converges in a reasonable period of time.

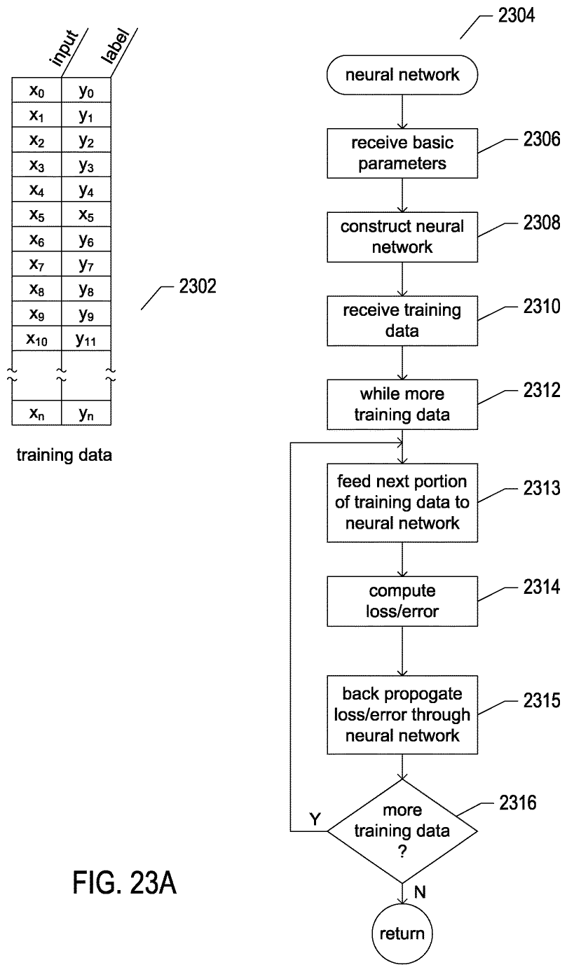

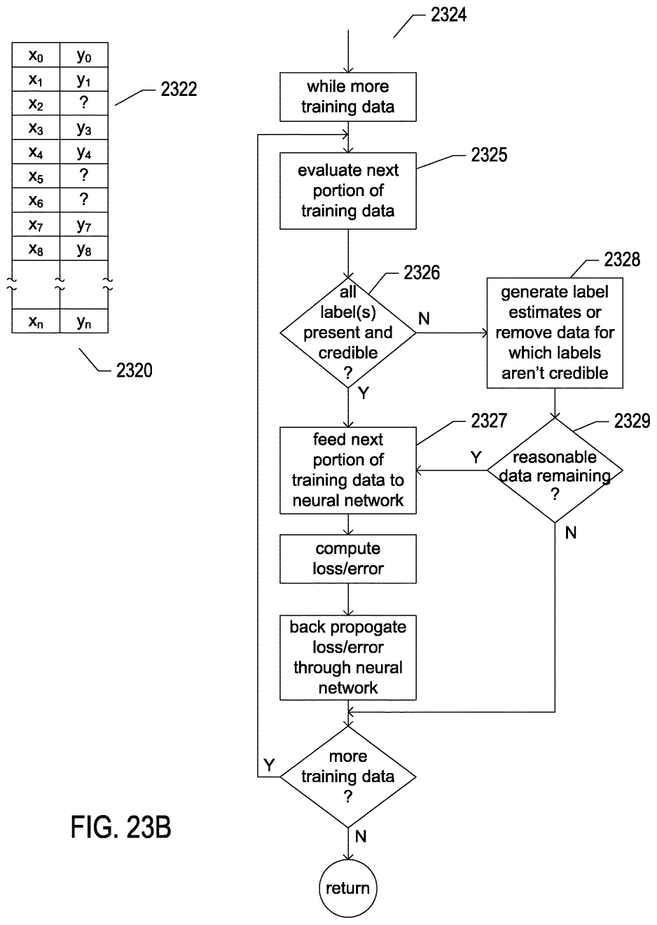

FIGS. 23A-B illustrate neural-network training. FIG. 23A illustrates the construction and training of a neural network using a complete and accurate training dataset. The training dataset is shown as a table of input-vector/label pairs 2302, in which each row represents an input-vector/label pair. The control-flow diagram 2304 illustrates construction and training of a neural network using the training dataset. In step 2306, basic parameters for the neural network are received, such as the number of layers, number of nodes in each layer, node interconnections, and activation functions. In step 2308, the specified neural network is constructed. This involves building representations of the nodes, node connections, activation functions, and other components of the neural network in one or more electronic memories and may involve, in certain cases, various types of code generation, resource allocation and scheduling, and other operations to produce a fully configured neural network that can receive input data and generate corresponding outputs. In many cases, for example, the neural network may be distributed among multiple computer systems and may employ dedicated communications and shared memory for propagation of activations and total error or loss between nodes. It should again be emphasized that a neural network is a physical system comprising one or more computer systems, communications subsystems, and often multiple instances of computer-instruction-implemented control components.

In step 2310, training data represented by table 2302 is received. Then, in the while-loop of steps 2312-2316, portions of the training data are iteratively input to the neural network, in step 2313, the loss or error is computed, in step 2314, and the computed loss or error is back-propagated through the neural network step 2315 to adjust the weights. The control-flow diagram refers to portions of the training data rather than individual input-vector/label pairs because, in certain cases, groups of input-vector/label pairs are processed together to generate a cumulative error that is back-propagated through the neural network. A portion may, of course, include only a single input-vector/label pair.

FIG. 23B illustrates one method of training a neural network using an incomplete training dataset. Table 2320 represents the incomplete training dataset. For certain of the input-vector/label pairs, the label is represented by a “?” symbol, such as in the input-vector/label pair 2322. The “?” symbol indicates that the correct value for the label is unavailable. This type of incomplete data set may arise from a variety of different factors, including inaccurate labeling by human annotators, various types of data loss incurred during collection, storage, and processing of training datasets, and other such factors. The control-flow diagram 2324 illustrates alterations in the while-loop of steps 2312-2316 in FIG. 23A that might be employed to train the neural network using the incomplete training dataset. In step 2325, a next portion of the training dataset is evaluated to determine the status of the labels in the next portion of the training data. When all of the labels are present and credible, as determined in step 2326, the next portion of the training dataset is input to the neural network, in step 2327, as in FIG. 23A. However, when certain labels are missing or lack credibility, as determined in step 2326, the input-vector/label pairs that include those labels are removed or altered to include better estimates of the label values, in step 2328. When there is reasonable training data remaining in the training-data portion following step 2328, as determined in step 2329, the remaining reasonable data is input to the neural network in step 2327. The remaining steps in the while-loop are equivalent to those in the control-flow diagram shown in FIG. 23A. Thus, in this approach, either suspect data is removed, or better labels are estimated, based on various criteria, for substitution for the suspect labels.

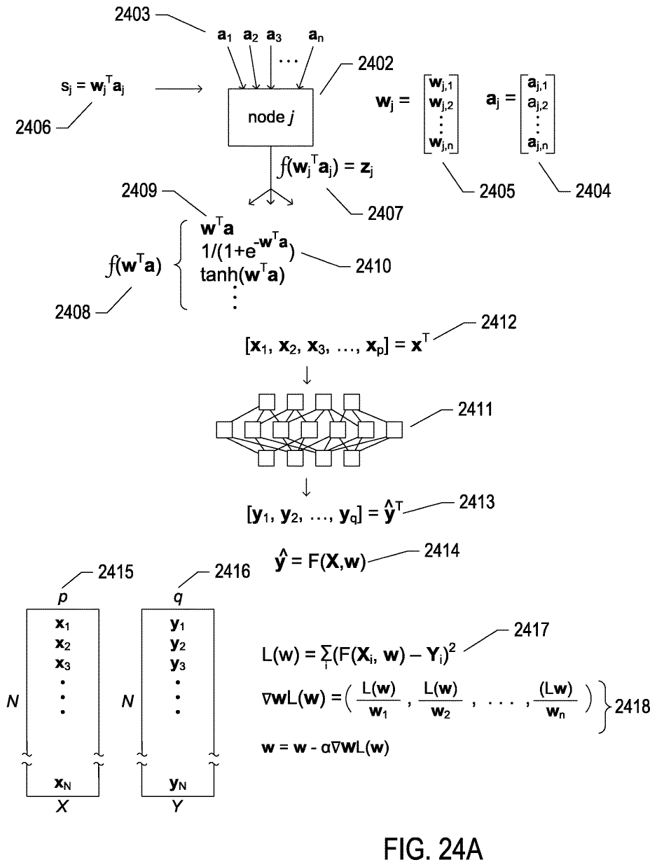

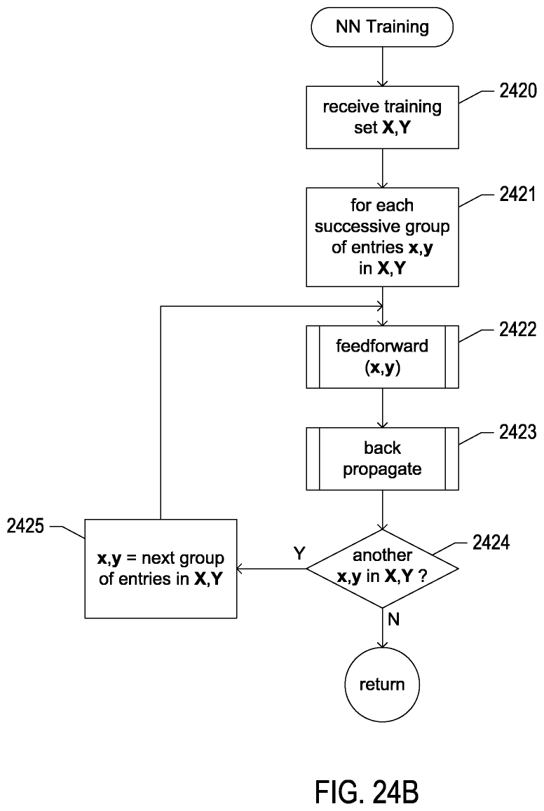

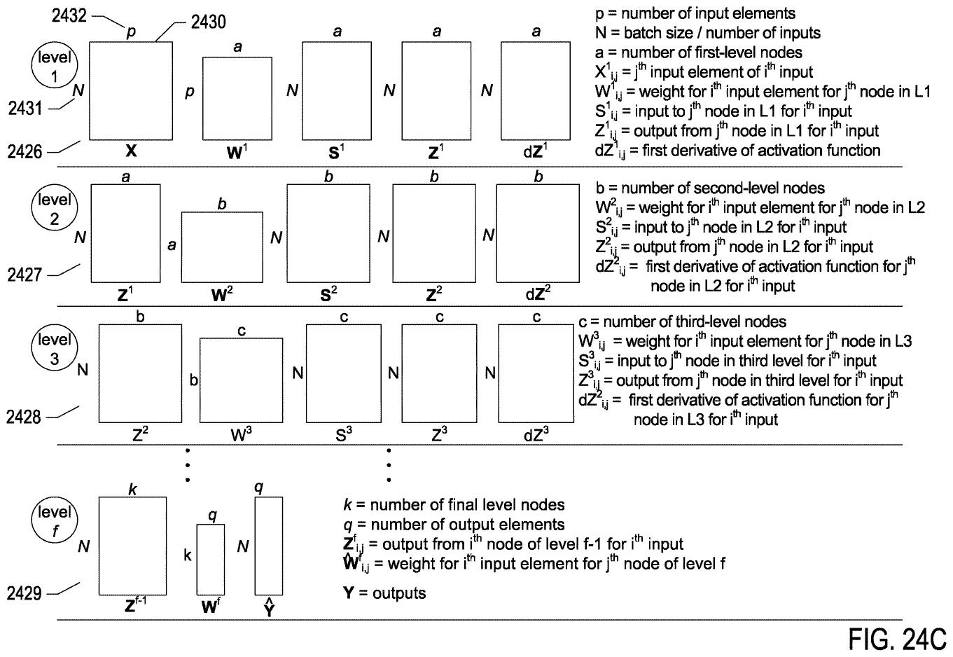

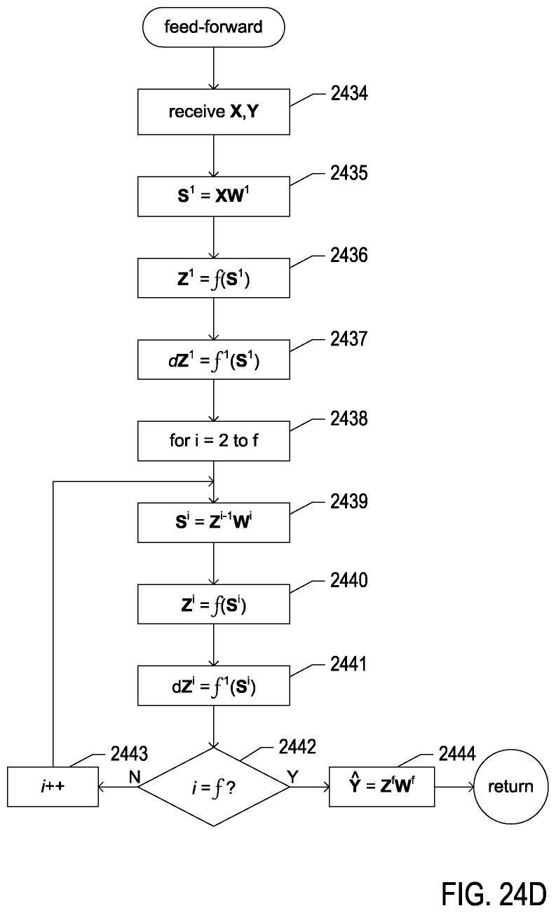

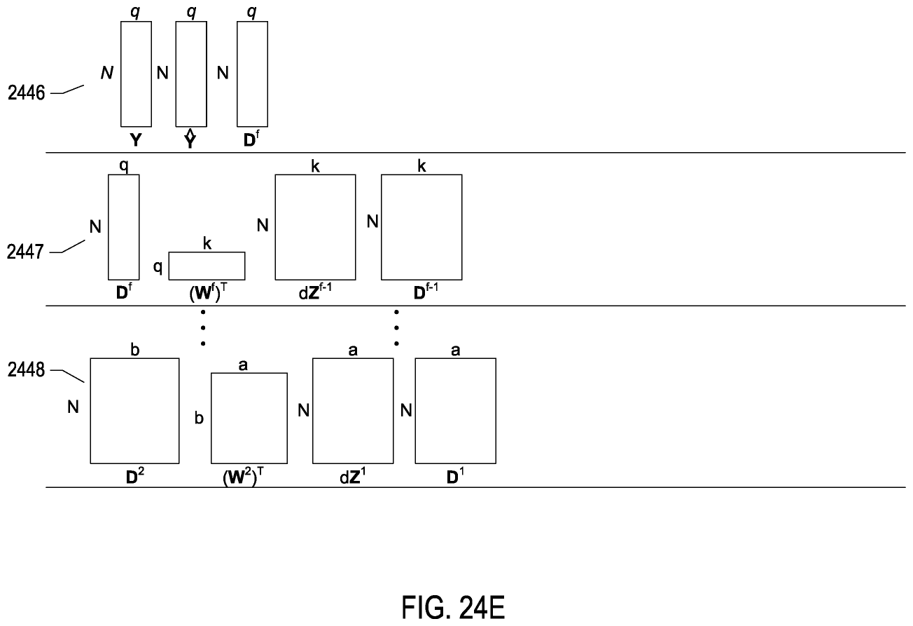

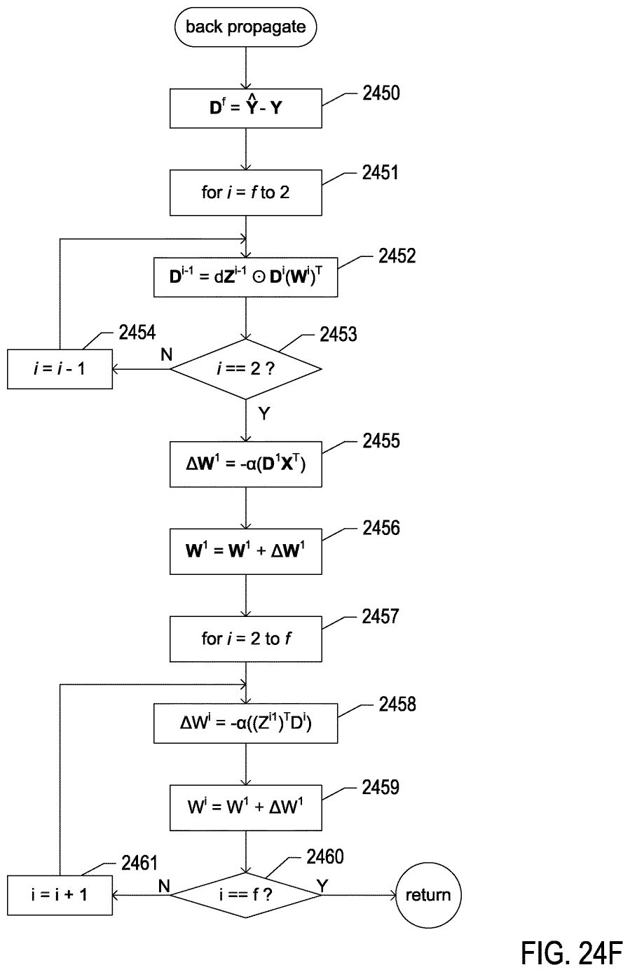

FIGS. 24A-F illustrate a matrix-operation-based batch method for neural-network training. This method processes batches of training data and losses to efficiently train a neural network. FIG. 24A illustrates the neural network and associated terminology. As discussed above, each node in the neural network, such as node j 2402, receives one or more inputs a 2403, expressed as a vector aⱼ 2404, that are multiplied by corresponding weights, expressed as a vector wⱼ 2405, and added together to produce an input signal sⱼ using a vector dot-product operation 2406. An activation function ƒ within the node receives the input signal sⱼ and generates an output signal zⱼ 2407 that is output to all child nodes of node j. Expression 2408 provides an example of various types of activation functions that may be used in the neural network. These include a linear activation function 2409 and a sigmoidal activation function 2410. As discussed above, the neural network 2411 receives a vector of p input values 2412 and outputs a vector of q output values 2413. In other words, the neural network can be thought of as a function F 2414 that receives a vector of input values x^{T} and uses a current set of weights w within the nodes of the neural network to produce a vector of output values ŷ^{T}. The neural network is trained using a training data set comprising a matrix X 2415 of input values, each of N rows in the matrix corresponding to an input vector x^{T}, and a matrix Y 2416 of desired output values, or labels, each of N rows in the matrix corresponding to a desired output-value vector y^{T}. A least-squares loss function is used in training 2417 with the weights updated using a gradient vector generated from the loss function, as indicated in expressions 2418, where α is a constant that corresponds to a learning rate.

FIG. 24B provides a control-flow diagram illustrating the method of neural-network training. In step 2420, the routine “NNTraining” receives the training set comprising matrices X and Y. Then, in the for-loop of steps 2421-2425, the routine “NNTraining” processes successive groups or batches of entries x and y selected from the training set. In step 2422, the routine “NNTraining” calls a routine “feedforward” to process the current batch of entries to generate outputs and, in step 2423, calls a routine “back propagated” to propagate errors back through the neural network in order to adjust the weights associated with each node.

FIG. 24C illustrates various matrices used in the routine “feedforward.” FIG. 24C is divided horizontally into four regions 2426-2429. Region 2426 approximately corresponds to the input level, regions 2427-2428 approximately correspond to hidden-node levels, and region 2429 approximately corresponds to the final output level. The various matrices are represented, in FIG. 24C, as rectangles, such as rectangle 2430 representing the input matrix X. The row and column dimensions of each matrix are indicated, such as the row dimension N 2431 and the column dimension p 2432 for input matrix X 2430. In the right-hand portion of each region in FIG. 24C, descriptions of the matrix-dimension values and matrix elements are provided. In short, the matrices W^{x} represent the weights associated with the nodes at level x, the matrices S^{x} represent the input signals associated with the nodes at level x, the matrices Z^{x} represent the outputs from the nodes at level x, and the matrices dZ^{x} represent the first derivative of the activation function for the nodes at level x evaluated for the input signals.

FIG. 24D provides a control-flow diagram for the routine “feedforward,” called in step 2422 of FIG. 24B. In step 2434, the routine “feedforward” receives a set of training data x and y selected from the training-data matrices X and Y. In step 2435, the routine “feedforward” computes the input signals S¹ for the first layer of nodes by matrix multiplication of matrices x and W¹, where matrix W¹ contains the weights associated with the first-layer nodes. In step 2436, the routine “feedforward” computes the output signals Z¹ for the first-layer nodes by applying a vector-based activation function ƒ to the input signals S¹. In step 2437, the routine “feedforward” computes the values of the derivatives of the activation function ƒ′, dZ¹. Then, in the for-loop of steps 2438-2443, the routine “feedforward” computes the input signals Sⁱ, the output signals Zⁱ, and the derivatives of the activation function dZⁱ for the nodes of the remaining levels of the neural network. Following completion of the for-loop of steps 2438-2443, the routine “feedforward” computes the output values ŷ^{T} for the received set of training data.

FIG. 24E illustrates various matrices used in the routine “back propagate.” FIG. 24E uses similar illustration conventions as used in FIG. 24C, and is also divided horizontally into horizontal regions 2446-2448. Region 2446 approximately corresponds to the output level, region 2447 approximately corresponds to hidden-node levels, and region 2448 approximately corresponds to the first node level. The only new type of matrix shown in FIG. 24E are the matrices D^{x} for node levels x. These matrices contain the error signals that are used to adjust the weights of the nodes.

FIG. 24F provides a control-flow diagram for the routine “back propagate.” In step 2450, the routine “back propagate” computes the first error-signal matrix D^{ƒ} as the difference between the values ŷ output during a previous execution of the routine “feedforward” and the desired output values from the training set y. Then, in a for-loop of steps 2451-2454, the routine “back propagate” computes the remaining error-signal matrices for each of the node levels up to the first node level as the Shur product of the dZ matrix and the product of the transpose of the W matrix and the error-signal matrix for the next lower node level. In step 2455, the routine “back propagate” computes weight adjustments ΔW for the first-level nodes as the negative of the constant α times the product of the transpose of the input-value matrix and the error-signal matrix. In step 2456, the first-node-level weights are adjusted by adding the current W matrix and the weight-adjustments matrix ΔW. Then, in the for-loop of steps 2457-2461, the weights of the remaining node levels are similarly adjusted.

Thus, as shown in FIGS. 24A-F, neural-network training can be conducted as a series of simple matrix operations, including matrix multiplications, matrix transpose operations, matrix addition, and the Shur product. Interestingly, there are no matrix inversions or other complex matrix operations needed for neural-network training.

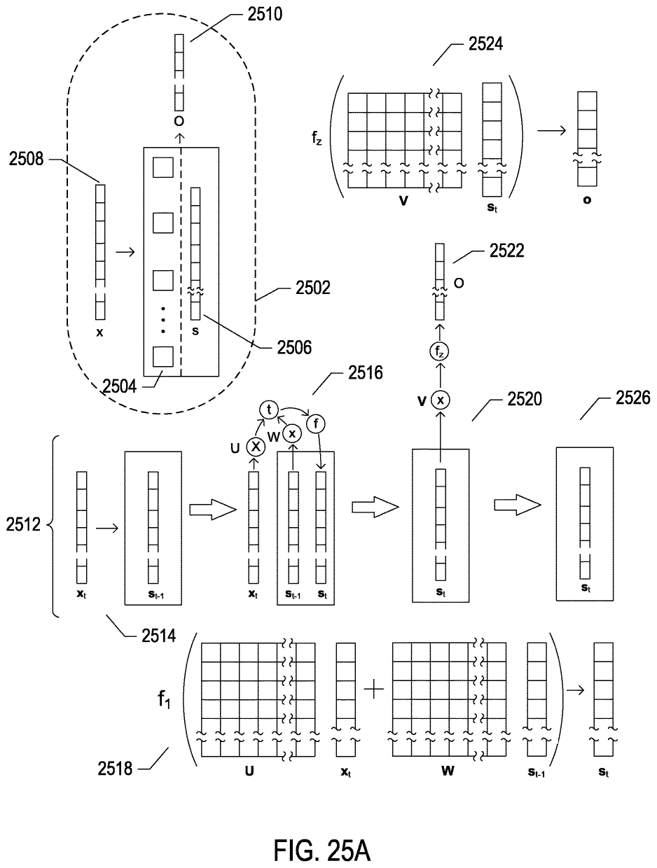

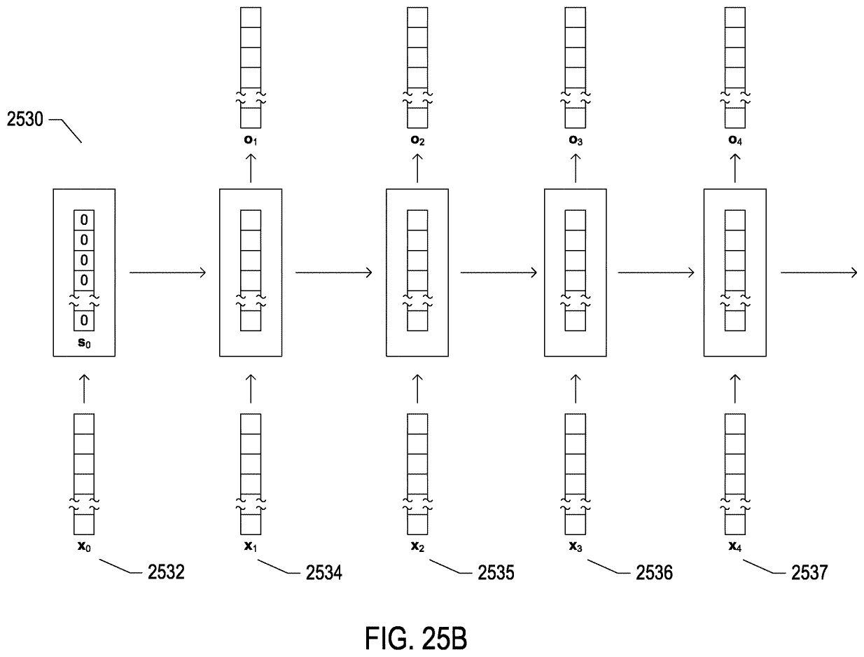

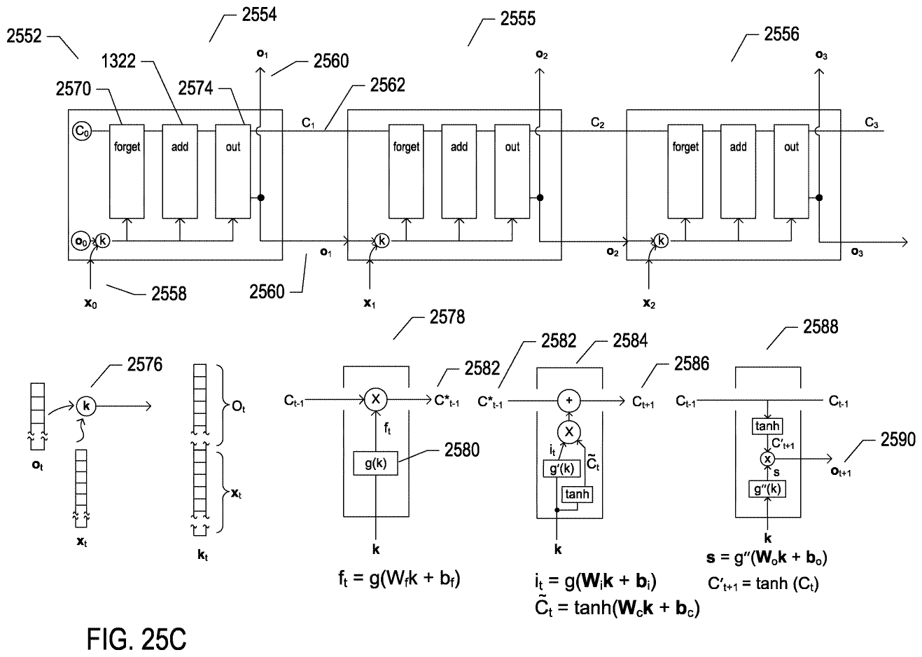

A second type of neural network, referred to as a “recurrent neural network,” is employed to generate sequences of output vectors from sequences of input vectors. These types of neural networks are often used for natural-language applications in which a sequence of words forming a sentence are sequentially processed to produce a translation of the sentence, as one example. FIGS. 25A-B illustrate various aspects of recurrent neural networks. Inset 2502 in FIG. 25A shows a representation of a set of nodes within a recurrent neural network. The set of nodes includes nodes that are implemented similarly to those discussed above with respect to the feed-forward neural network 2504, but additionally include an internal state 2506. In other words, the nodes of a recurrent neural network include a memory component. The set of recurrent-neural-network nodes, at a particular time point in a sequence of time points, receives an input vector x 2508 and produces an output vector 2510. The process of receiving an input vector and producing an output vector is shown in the horizontal set of recurrent-neural-network-nodes diagrams interleaved with large arrows 2512 in FIG. 25A. In a first step 2514, the input vector x at time t is input to the set of recurrent-neural-network nodes which include an internal state generated at time t−1. In a second step 2516, the input vector is multiplied by a set of weights U and the current state vector is multiplied by a set of weights W to produce two vector products which are added together to generate the state vector for time t. This operation is illustrated as a vector function ƒ₁ 2518 in the lower portion of FIG. 25A. In a next step 2520, the current state vector is multiplied by a set of weights V to produce the output vector for time t 2522, a process illustrated as a vector function ƒ₂ 2524 in FIG. 25A. Finally, the recurrent-neural-network nodes are ready for input of a next input vector at time t+1, in step 2526.

FIG. 25B illustrates processing by the set of recurrent-neural-network nodes of a series of input vectors to produce a series of output vectors. At a first time to 2530, a first input vector x₀ 2532 is input to the set of recurrent-neural-network nodes. At each successive time point 2534-2537, a next input vector is input to the set of recurrent-neural-network nodes and an output vector is generated by the set of recurrent-neural-network nodes. In many cases, only a subset of the output vectors is used. Back propagation of the error or loss during training of a recurrent neural network is similar to back propagation for a feed-forward neural network, except that the total error or loss needs to be back-propagated through time in addition to through the nodes of the recurrent neural network. This can be accomplished by unrolling the recurrent neural network to generate a sequence of component neural networks and by then backpropagating the error or loss through this sequence of component neural networks from the most recent time to the most distant time period.

Finally, for completeness, FIG. 25C illustrates a type of recurrent-neural-network node referred to as a long-short-term-memory (“LSTM”) node. In FIG. 25C, a LSTM node 2552 is shown at three successive points in time 2554-2556. State vectors and output vectors appear to be passed between different nodes, but these horizontal connections instead illustrate the fact that the output vector and state vector are stored within the LSTM node at one point in time for use at the next point in time. At each time point, the LSTM node receives an input vector 2558 and outputs an output vector 2560. In addition, the LSTM node outputs a current state 2562 forward in time. The LSTM node includes a forget module 2570, an add module 2572, and an out module 2574. Operations of these modules are shown in the lower portion of FIG. 25C. First, the output vector produced at the previous time point and the input vector received at a current time point are concatenated to produce a vector k 2576. The forget module 2578 computes a set of multipliers 2580 that are used to element-by-element multiply the state from time t−1 in order to produce an altered state 2582. This allows the forget module to delete or diminish certain elements of the state vector. The add module 2134 employs an activation function to generate a new state 2586 from the altered state 2582. Finally, the out module 2588 applies an activation function to generate an output vector 2140 based on the new state and the vector k. An LSTM node, unlike the recurrent-neural-network node illustrated in FIG. 25A, can selectively alter the internal state to reinforce certain components of the state and deemphasize or forget other components of the state in a manner reminiscent of human short-term memory. As one example, when processing a paragraph of text, the LSTM node may reinforce certain components of the state vector in response to receiving new input related to previous input but may diminish components of the state vector when the new input is unrelated to the previous input, which allows the LSTM to adjust its context to emphasize inputs close in time and to slowly diminish the effects of inputs that are not reinforced by subsequent inputs. Here again, back propagation of a total error or loss is employed to adjust the various weights used by the LSTM, but the back propagation is significantly more complicated than that for the simpler recurrent neural-network nodes discussed with reference to FIG. 25A.

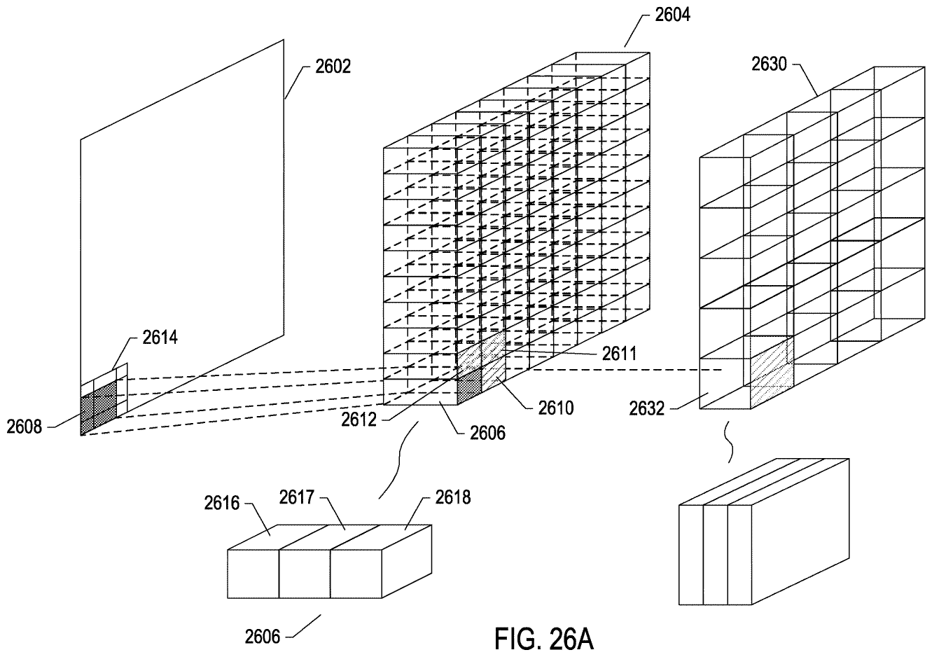

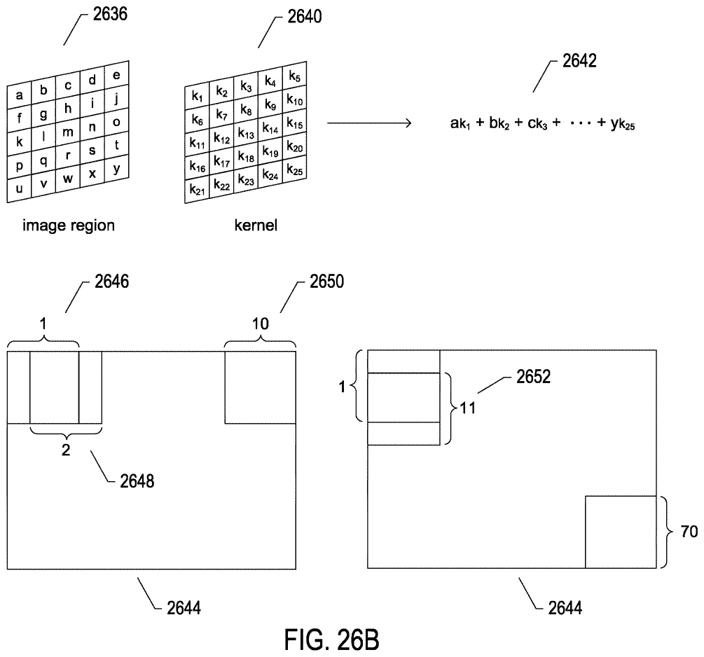

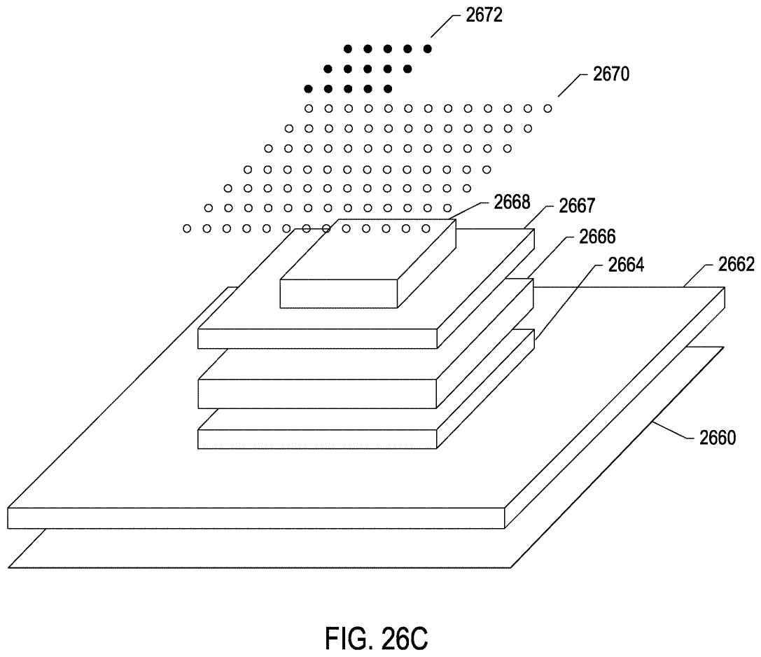

Figure FIGS. 26A-C illustrate a convolutional neural network. Convolutional neural networks are currently used for image processing, voice recognition, and many other types of machine-learning tasks for which traditional neural networks are impractical. In FIG. 26A, a digitally encoded screen-capture image 2602 represents the input data for a convolutional neural network. A first level of convolutional-neural-network nodes 2604 each process a small subregion of the image. The subregions processed by adjacent nodes overlap. For example, the corner node 2606 processes the shaded subregion 2608 of the input image. The set of four nodes 2606 and 2610-2612 together process a larger subregion 2614 of the input image. Each node may include multiple subnodes. For example, as shown in FIG. 26A, node 2606 includes 3 subnodes 2616-2618. The subnodes within a node all process the same region of the input image, but each subnode may differently process that region to produce different output values. Each type of subnode in each node in the initial layer of nodes 2604 uses a common kernel or filter for subregion processing, as discussed further below. The values in the kernel or filter are the parameters, or weights, that are adjusted during training. However, since all the nodes in the initial layer use the same three subnode kernels or filters, the initial node layer is associated with only a comparatively small number of adjustable parameters. Furthermore, the processing associated with each kernel or filter is more or less translationally invariant, so that a particular feature recognized by a particular type of subnode kernel is recognized anywhere within the input image that the feature occurs. This type of organization mimics the organization of biological image-processing systems. A second layer of nodes 2630 may operate as aggregators, each producing an output value that represents the output of some function of the corresponding output values of multiple nodes in the first node layer 2604. For example, a second-layer node 2632 receives, as input, the output from four first-layer nodes 2606 and 2610-2612 and produces an aggregate output. As with the first-level nodes, the second-level nodes also contain subnodes, with each second-level subnode producing an aggregate output value from outputs of multiple corresponding first-level subnodes.

FIG. 26B illustrates the kernel-based or filter-based processing carried out by a convolutional neural network node. A small subregion of the input image 2636 is shown aligned with a kernel or filter 2640 of a subnode of a first-layer node that processes the image subregion. Each pixel or cell in the image subregion 2636 is associated with a pixel value. Each corresponding cell in the kernel is associated with a kernel value, or weight. The processing operation essentially amounts to computation of a dot product 2642 of the image subregion and the kernel, when both are viewed as vectors. As discussed with reference to FIG. 26A, the nodes of the first level process different, overlapping subregions of the input image, with these overlapping subregions essentially tiling the input image. For example, given an input image represented by rectangles 2644, a first node processes a first subregion 2646, a second node may process the overlapping, right-shifted subregion 2648, and successive nodes may process successively right-shifted subregions in the image up through a tenth subregion 2650. Then, a next down-shifted set of subregions, beginning with an eleventh subregion 2652, may be processed by a next row of nodes.

FIG. 26C illustrates the many possible layers within the convolutional neural network. The convolutional neural network may include an initial set of input nodes 2660, a first convolutional node layer 2662, such as the first layer of nodes 2604 shown in FIG. 26A, and aggregation layer 2664, in which each node processes the outputs for multiple nodes in the convolutional node layer 2662, and additional types of layers 2666-2668 that include additional convolutional, aggregation, and other types of layers. Eventually, the subnodes in a final intermediate layer 2668 are expanded into a node layer 2670 that forms the basis of a traditional, fully connected neural-network portion with multiple node levels of decreasing size that terminate with an output-node level 2672.

### The Currently Disclosed Methods and Systems

As discussed in preceding sections of this document, there are a number of different therapeutic methods that have been devised for stimulating different types of electrical activity in the human brain in order to ameliorate numerous different conditions, including depression, obsessive-compulsive disorder, schizophrenia, chronic pain, and various additional neurological and psychiatric conditions. These therapeutic methods include the above-discussed transcranial direct current stimulation (“tDCS”), transcranial magnetic stimulation (“TMS”), additional related methods, including transcranial alternating current stimulation (“tACS”), stimulation via various types of sensory input, drug-based treatments, and surgical interventions. However, the physiology, molecular biology, and dynamics of brain function and mental processes, despite decades if not centuries of research, are still not well understood. As a result, it remains difficult or impossible to accurately and deterministically select, plan, and develop effective treatments for specific conditions manifested in specific individuals. Currently, treatment selection and planning are largely empirical and often essentially hit-or-miss. However, unlike research conducted on lab rodents or other such subjects, treatment planning and selection based on experimentation are burdensome and expensive for patients and for practitioners. Furthermore, it may actually be detrimental to unnecessarily expose patients to various types of stimulation and other treatments in order to attempt to find effective treatments and treatment plans. For these reasons, treatment providers and patients have recognized the need for more cost-effective, safer, and more time-efficient treatment-selection and treatment-planning methods.

The currently disclosed methods and systems are intended to address the problems mentioned in the preceding paragraph. In particular, the currently disclosed methods and systems feature development of a personalized computational model for a patient's brain-electrical-activity response to application of transcranial electrical or magnetic stimuli to selected brain regions as well as a method to calibrate this computational model based on patient observations. This computational model can be used to facilitate time-efficient, cost-effective, and safe selection of treatments and development of specific treatment plans with minimum experimentation. The currently disclosed methods and systems are thus well-targeted to addressing current problems in treating neurological and psychiatric conditions and will be important in advancing the treatment of neurological and psychiatric conditions and disorders. Many of the considerations and innovations involved with, and incorporated in, the methods and systems disclosed in this document can be more generally applied to a wide variety of different types of conditions and disorders, monitoring and treatment methodologies and instrumentation, and treatment planning.

FIGS. 27A-B provide a control-flow diagram for a routine “treatment determination,” which illustrates significant features of the currently disclosed methods and systems. In step 2702, the routine “treatment determination” receives initial patient data and other information needed for applying a stimulus/therapeutic input to the patient. When this information includes magnetic resonance imaging (“MRI”) data or other anatomical data, as determined in step 2704, the anatomical data, EEG data collected from the patient, and/or other observations are used to construct an initial computational model referred to as a “whole-brain network model” (“WBNM”), in step 2706. Otherwise, EEG data collected from the patient and/or other observations are used to generate the WBNM, in step 2708. The WBNM is discussed, in detail, below. Following generation of the WBNM, the WBNM is calibrated or optimized, in step 2710. This step involves minimizing the differences between EEG signals observed in the patient and simulated EEG signals output by the WBNM. The optimization process is discussed, in detail, below. In step 2712, the routine “treatment determination” generates a modified WBNM (“mWBNA”) by modifying certain of the differential equations that form a basis for the WBNM. This modification is discussed, in detail, below. Then, in step 2714, the mWBNM is optimized, as further discussed below. Following completion of step 2714, a computational model needed for treatment-plan determination is in hand. In step 2716, the mWBNM is used to determine a set of control parameters for applying the stimulus/therapeutic input to the patient by finding a set of control parameters that minimize a severity level generated from simulated EEG data and/or other simulated observations following simulated application of the stimulus/therapeutic input to the patient. Note that the severity level may be computed for a specific neurological or psychiatric condition or may be a more generic indication of neurological health. In other words, the currently disclosed methods and systems use the computational model to find an initial set of control parameters. Control parameters vary for different types of stimulus and different types of devices and systems used to apply stimuli. They are parameters that define the applied stimuli, including duration, frequency, signal strength, application location or locations, and other such parameters. This is, of course, far more cost-effective, time-efficient, and safer than carrying out actual experiments on the patient in order to determine a set of control parameters. Currently, when an experimental approach is used to determine an initial set of control parameters, the time delays between experimental application of stimuli/therapeutic inputs and determination of the effects of those stimuli/therapeutic inputs can be relatively long and imprecise, often involving oral or written patient feedback and/or lengthy patient observations. Were an optimized mWBNM not available for generating simulated EEG data, step 2716 would likely not result in cost-effectiveness, time efficiency, or safety. It is the combination of steps 2708, 2710, 2712, 2714, and 2716 that provides the initial improvements to treatment planning with respect to currently available methods.

With the initial computational generated treatment parameters in hand, the routine “treatment determination” then can undertake limited experimentation, starting in step 2718, to improve the parameters/treatment. In step 2718, local variable num is set to the maximum number of stimulus/therapeutic-input applications that can be carried out on the particular patient. This number may be determined, in part, from the initially received patient data. Local variable best_control_parameters is set to the list of control-parameter values determined in step 2716. Local variable cur_control_parameters is set to the same list of control-parameter values. Local variable best_treatment_efficiency is set to a large numerical value maxF. A numerical value representing treatment efficacy is 0 for maximum efficiency and increases as the represented efficiency decreases. Finally, a patient-specific in vivo model IVM that returns an estimated treatment efficacy for an input set of control parameters is generated, as further discussed below.

Turning to FIG. 27B, the routine “treatment determination” begins to execute the while-loop of steps 2720-2727, which iterates until the value stored in local variable num falls below 1. When the value stored in local variable num is greater than 1, as determined in step 2720, the routine “treatment determination” uses the parameter values stored in local variable cur_control_parameters to apply a stimulus/therapeutic input to the patient in step 2721. In step 2722, the severity level exhibited by the patient following application of the stimulus/therapeutic input is determined using EEG and/or other observational methods, allowing the routine “treatment determination” to determine the current treatment efficacy associated with the parameter values contained in local variable cur_control_parameters. When the determined current treatment efficacy has a value less than the value stored in local variable best_treatment_efficiency, as determined in step 2723, local variable best_treatment_efficiency is set to the current treatment efficacy determined in step 2722 and local variable best_control_parameters is set to the parameter values stored in local variable cur_control_parameters in step 2724. In step 2725, local variable num is decremented. In step 2726, the current treatment efficacy and IVM are used to generate a new in vivo model newIVM. The new model newIVM is then used to generate a new set of control parameters that are stored in local variable cur_control_parameters. When local variable num contains a value greater than 1, as determined in step 2727, control returns to step 2721 for a next iteration of the while-loop of steps 2720-2727. Otherwise control flows to step 2728. When the parameter “mode” is not equal to conservative, as determined in step 2728, control flows to step 2730 in which the routine “treatment determination” stores the control values contained in local variable cur_control_parameters for the patient for subsequent use in treatments and treatment planning and then uses these control parameters to apply a therapeutic stimulus to the patient. When the parameter “mode” is equal to conservative, as determined in step 2728 and when local variable best_treatment_efficiency is equal to maxF, as determined in step 2732, indicating that there have been no improvements made to the control parameters determined in step 2716, control flows to step 2730 for application of a therapeutic stimulus to the patient and storing of the current control parameters for the patient. Otherwise, in step 2734, the routine “treatment determination” stores the control parameters contained in local variable best_control_parameters for subsequent use and uses these control parameters to apply a therapeutic stimulus to the patient. Thus, the routine “treatment determination” both computationally estimates control parameters for applying a stimulus/therapeutic input to the patient and can then refine the initial control parameters by limited experimentation. Ultimately, whether using the initially computed control parameters or refining control parameters through limited experimentation, the routine “treatment determination” applies a therapeutic stimulus to the patient in addition to storing the control parameters used to apply the therapeutic stimulus for future reference. Thus, the currently disclosed methods not only use the mWBNM to compute initial control parameters and thus provide a cost-effective, time-efficient, and safe approach to treatment planning, but also allow for limited experimentation in order to refine the control parameters and provide the most accurate therapeutic stimulus based on limited experimentation.

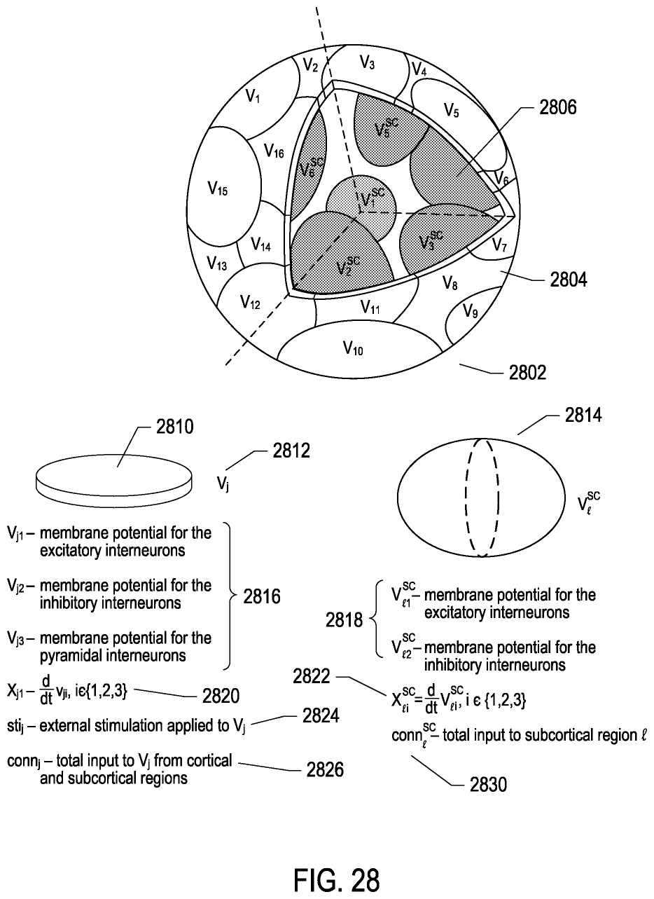

FIGS. 28-31 provide a complete description of the mathematical model underlying the WBNM. The mathematical model is, like the above-discussed Jansen-Rit neural mass model, defined by a system of coupled differential equations. The coupled differential equations are solved by various different types of methods, including matrix methods that can be used to determine eigenvalues and eigenvectors that are used, in turn, to produce a computational model for computing the membrane potentials of the neuron populations at a time t resulting from external inputs to the system. Thus, the acronym “WBNM,” in the current discussion, refers to a computational model that can be initialized with parameter values and executed, on one or more computer systems, to produce numerical values for the membrane potentials of the neuronal populations of the cortical regions and subcortical regions. The numerical values for membrane potentials produced by the WBNM depend on the values of various different parameters, discussed below, in addition to external signal inputs. The WBNM can be used to step through successive time points within a time interval to generate the membrane potentials for the neuronal populations at each time point, which leads to a dynamic model of electrical activity within the brain. This, in turn, can be used to generate simulated EEG signals corresponding to the dynamic model of electrical activity, as discussed above and further discussed below.

FIG. 28 illustrates general concepts and certain notation related to the whole-brain network model (“WBNM”). Sphere 2802 is an abstract representation of the human brain. It contains a thin outer shell representing the cerebral cortex 2804 and shaded internal volumes representing subcortical regions of the human brain, including subcortical region 2806. Of course, the human brain is not a sphere and the subcortical regions are not simple spherical or ellipsoidal volumes, but the simple abstract diagram 2802 is useful in describing nomenclature used in the WBNM. A set of membrane potentials for each cortical region 2010 is denoted by the notation vⱼ 2812, where j is an index unique to the cortical region. A set of membrane potentials for each subcortical region 2814 is denoted by the notation vₗ^{SC}, where l is an index unique to the subcortical region. Second indices are used to indicate particular populations of neurons within the regions: (1) second index 1 indicates excitatory interneurons; (2) second index 2 indicates inhibitory neurons; and (3) second index 3 indicates pyramidal neurons. The notations for the specific neuron populations are shown in expressions 2816 and 2818. Similar notation is used for the first derivative with respect to time of the region-specific membrane potentials, with the symbol “x” replacing the symbol “v,” as shown in expressions 2820 and 2822. The notation stiⱼ denotes the external stimulation applied to a cortical region j, as indicated by expression 2824. External stimulation includes therapeutic stimuli such as the above-discussed tDCS and TMS stimuli. Finally, notations for the total input to cortical and subcortical regions replace symbol “v” with “conn” and use a single region index, as indicated by expressions 2826 and 2830.

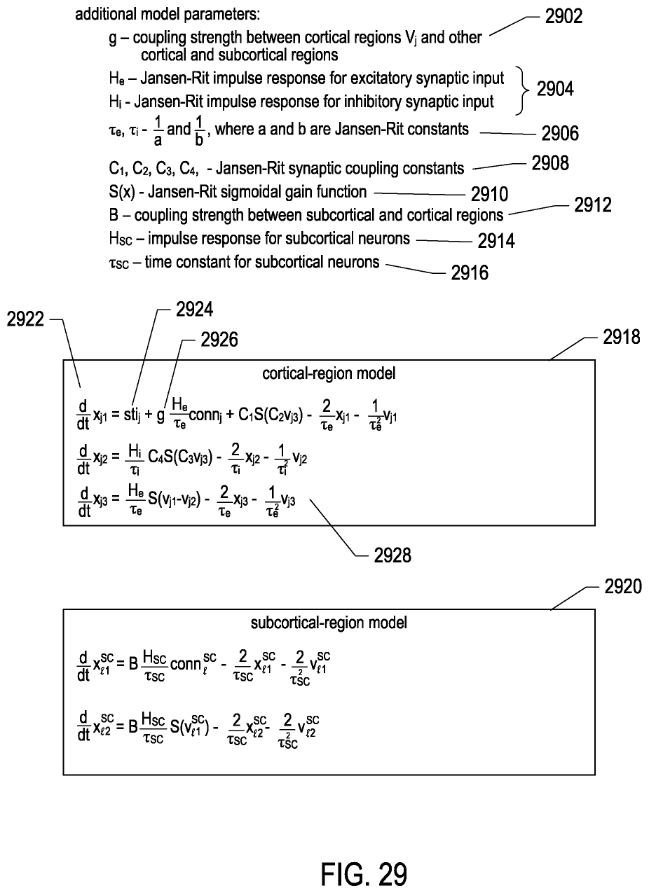

FIG. 29 lists additional WBNM model parameters and provides the coupled differential equations that together comprise the basis of the WBNM. The additional model parameters include a coupling strength between cortical regions and other cortical and subcortical regions 2902, the above-discussed Jansen-Rit impulse responses for auditory and inhibitory synaptic inputs 2904, two time constants derived from Jansen-Rit constants 2906, the Jansen-Rit synaptic coupling constants 2908, the Jansen-Rit sigmoidal gain function 2910, a coupling strength between subcortical and cortical regions 2912, an impulse response for subcortical neurons 2914, and a time constant for subcortical neurons 2916. Three second-order differential equations similar to the Jansen-Rit differential equations together comprise the cortical-region portion of the WBNM 2918 and two second-order differential equations similar to the Jansen-Rit differential equations together comprise the subcortical-region portion of the WBNM 2920. Differential equation 2922 is similar to the first of the differential equations of the Jansen-Rit model in the set of equations 1656 shown in FIG. 16C. However, the WBNM equation includes a term for the external stimulus 2924 and a term for the input from other cortical and subcortical regions 2926. The final two differential equations in the set of equations 1656 in FIG. 16C are combined to generate equation 2928 and certain terms are distributed differently in the two sets of equations. Importantly, unlike the previously discussed Jansen-Rit model, the WBNM includes the effects of synchronization between brain regions, encapsulated in the conn terms representing total input from other cortical and subcortical regions, as discussed below, and the WBNM considers the effects of external therapeutic stimuli and other external inputs. Furthermore, the WBNM includes a separate model portion for subcortical regions.

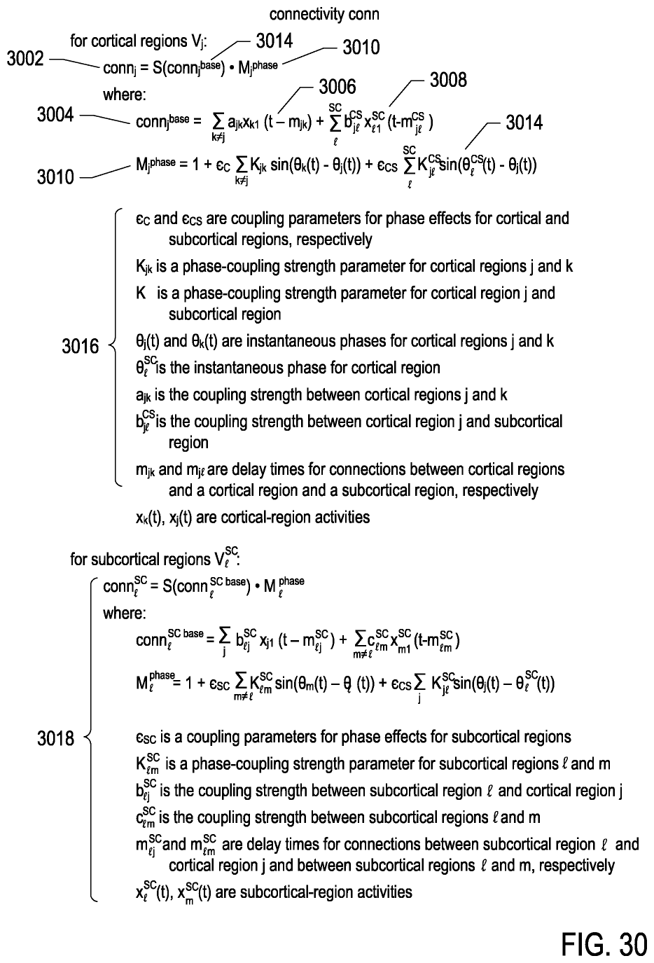

FIG. 30 provides details for the connectivity terms connⱼ and connₗ^{SC} in the cortical-region model portion 2918 and the subcortical-region model portion 2920 in FIG. 29. The connⱼ term 3002 represents the total input to a cortical region and includes a cumulative firing rate 3004 that includes firing-rate contributions from other cortical regions 3006 and firing-rate contributions from subcortical regions 3008. The cumulative firing rate term 3002 is multiplied by a phase-coupling-modulation term 3010 that considers synchronization between brain regions according to the above-discussed Kuramoto module, where the synchronization considers other cortical regions 3012 and subcortical regions 3014. The definitions and explanations for the various terms used in the expressions for the connectivity term connⱼ are provided in expressions 3016. Expressions 3018 define the connₗ^{SC} connectivity term for the subcortical regions.

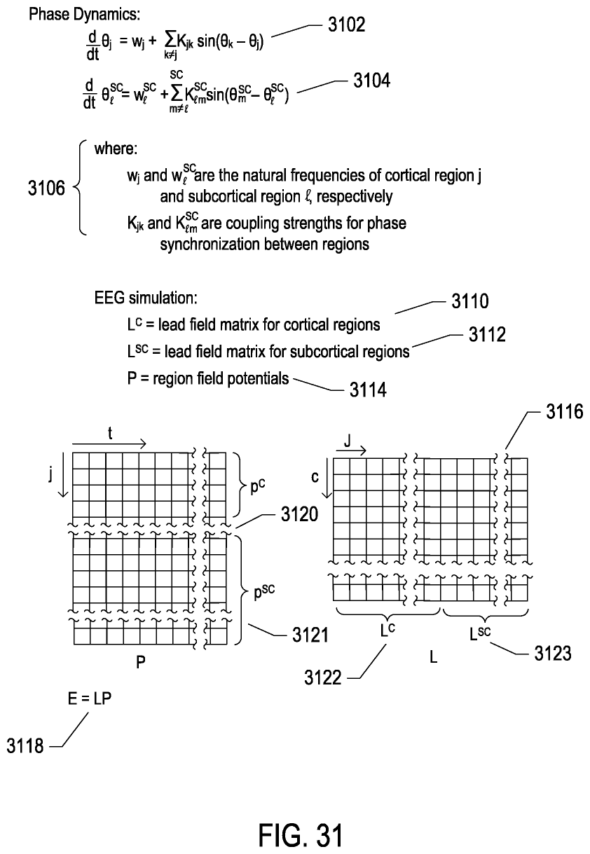

FIG. 31 illustrates the phase dynamics incorporated into the above-discussed connectivity terms. A first expression 3102 represents a series of coupled differential equations for synchronization of the cortical regions and a second expression 3104 represents a set of coupled differential equations for the subcortical regions. These expressions are directly related to the above-discussed Kuramoto model and the various terms and constants used in the coupled differential equations are defined in expressions 3106. Finally, in the lower portion of FIG. 31, the overall approach to simulating EEG signals from the WBNM is illustrated. As discussed above with reference to FIGS. 12B-C, given a lead-field matrix for the cortical regions 3110, a lead-field matrix for the subcortical regions 3112, and a P matrix of region membrane potentials 3114 generated by computing the membrane potentials of the neuron populations of cortical and subcortical regions for successive time points using a WBNM, a simulated EEG signal is generated by multiplying the P matrix from the left by the L matrix 3118, where the L matrix is obtained as a horizontally partitioned matrix by combining the lead-field matrices 3120-3120 for the cortical and subcortical regions and the P matrix is a vertically partitioned matrix that includes two submatrices 3122-3123 corresponding to the membrane potentials generated for the cortical and subcortical regions.

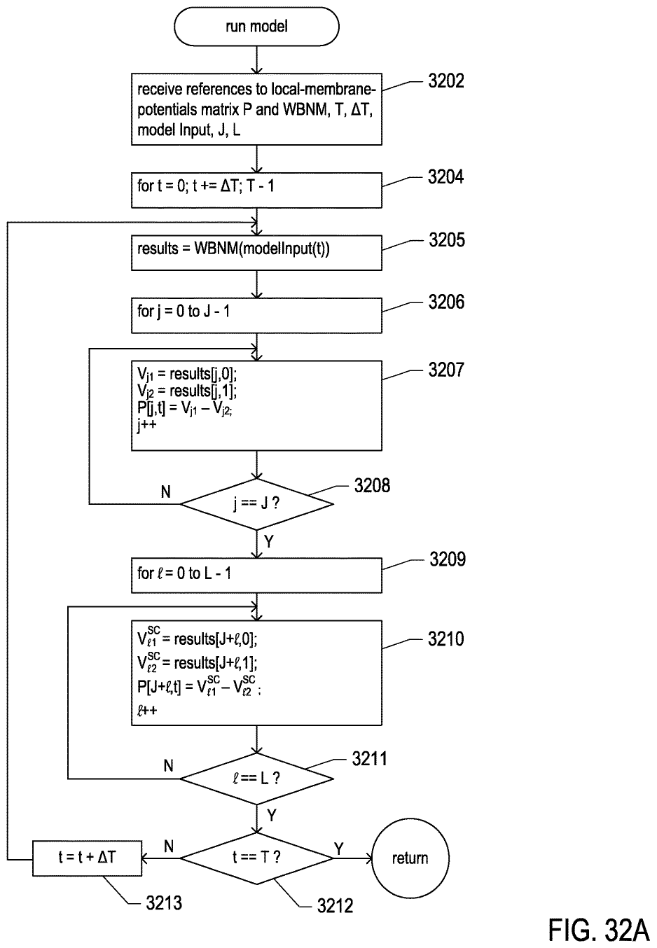

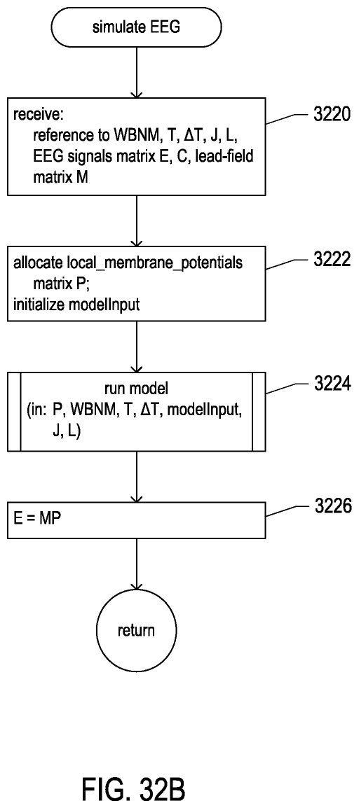

FIGS. 32A-B provide control-flow diagrams for a routine “simulate EEG” that generates simulated EEG signals observed for a patient for whom a WBNM has been generated. In general, a modified optimized WBNM is used in treatment planning according to the currently disclosed methods and systems, but the initial WBNM, optimized WBNM, and the modified optimized WBNM can all be used to generate simulated EEG signals. FIG. 32A provides a control-flow diagram for a routine “run model” that generates membrane potentials for cortical and subcortical regions for each time point within a time interval, storing the membrane potentials in a local-membrane-potentials matrix P, discussed above with reference to FIG. 12C. In step 3202, the routine “run model” receives a reference to a local-membrane-potentials matrix P, a reference to a WBNM, the number of time steps Tin the time interval, the length of time between time steps ΔT, a reference to a function modelInput, the number of cortical regions J, and the number of subcortical regions L. In the outer for-loop of steps 3204-3213, the routine “run model” considers each time point t in the time interval. In step 3205, the routine “run model” inputs the current input signals, obtained by calling the function modelInput with the currently considered time point, to the WBNM, which returns the membrane potentials for the neuron populations in each of the cortical and subcortical brain regions in the anatomical model incorporated into the WBNM. The returned membrane potentials are stored in local variable results. Then, in the inner for-loop of steps 3206-3208, each cortical region indexed by index j is considered. In step 3707, for the currently considered cortical region j, the excitatory and inhibitory interneuron membrane potentials are extracted from the local variable results and the inhibitory membrane potential is subtracted from the excitatory membrane potential to generate the cumulative membrane potential for cortical region j, which is stored into the matrix P. The inner for-loop iterates until all of the cortical regions have been considered, as determined in step 3208. Similarly, in a second inner for-loop of steps 3209-3211, the cumulative membrane potentials for subcortical regions are determined and stored in the matrix P. The second inner for-loop iterates until all of the subcortical regions have been considered, as determined in step 3211. The outer for-loop of steps 3204-3213 continues until, as determined in step 3212, all of the time steps in the time interval have been considered. The current time point t is incremented prior to the beginning of a next iteration of the outer for-loop in step 3213.

FIG. 32B provides a control-flow diagram for the routine “simulate EEG” which generates simulated EEG signals for a time interval using a WBNM. In step 3220, the routine “simulate EEG” receives a reference to the WBNM, the number of time points in the time interval T, the length of time between time points ΔT, the number of cortical regions J, the number of subcortical regions L, a reference to an EEG signals matrix E, the number of channels c in the matrix E, and a lead-field matrix M (note that “M” is used instead of “L” to avoid confusion with the number of subcortical regions L). In step 3222, the routine “simulate EEG” allocates a local-membrane-potentials matrix P and initializes the function modelInput. As mentioned above, this function returns the input signals to the WBNM for each time point in the time interval. The input signals may include therapeutic stimulus inputs, when the simulated EEG is desired for evaluating a severity level, and generally includes some type of background signal representing normal brain activity. In step 3224, the routine “simulate EEG” calls the routine “run model,” discussed above with reference to FIG. 32A, to generate the local membrane potentials generated by the WBNM for the time interval and store them in the matrix P. The time series of local membrane potentials often include oscillating components that reflect synchronized depolarization and hyperpolarization of interneurons in two or more brain regions. Finally, in step 3226, the matrix P is multiplied from the left by the lead-field matrix M to produce signals that are stored in the EEG signals matrix E, as discussed above with reference to FIG. 31.

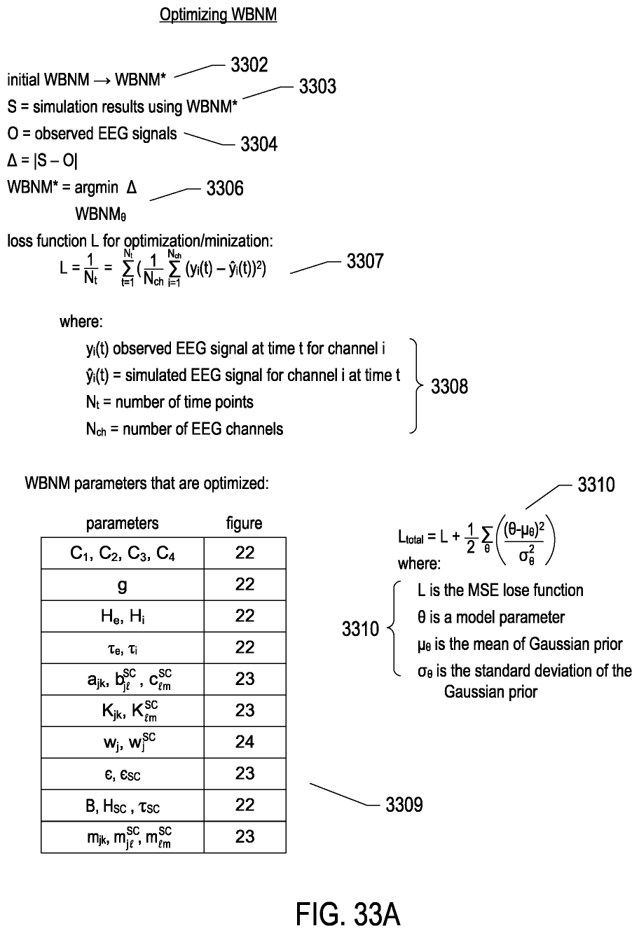

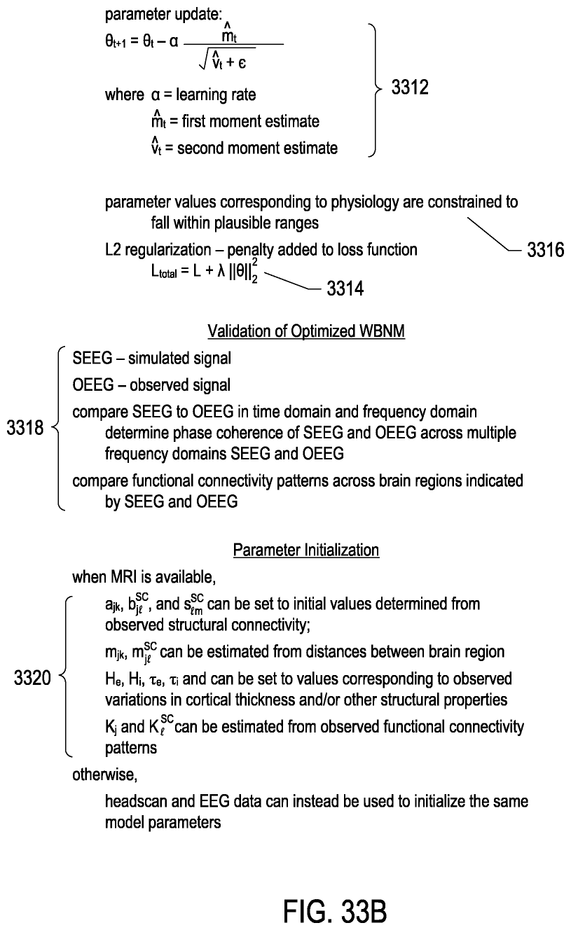

FIGS. 33A-B illustrate calibration or optimization of the WBNM mentioned above with respect to step 2710 in FIG. 27A. The calibration or optimization step receives an initial WBNM and returns an optimized WBNM* that more accurately reproduces observed EEG signals 3302. Given that the symbol “S” denotes simulation results obtained from the optimized WBNM* 3303, “O” denotes observed EEG signals from the patient 3304, and A denotes a computed difference magnitude between the simulation results and observed signals 3305, the calibration or optimization step seeks to minimize A with respect to the WBNM* parameters 3306. A mean-squared-error loss function L for the optimization is shown as expression 3307, the loss function computed over the time points of an interval and over all EEG-signal channels. Expressions 3308 defines the variables and constants used in expression 3307. Table 3309 lists, in a first column, the parameters that may be optimized in the currently disclosed calibration step, with a figure number shown, in a second column, for each parameter to indicate where the parameter is illustrated and explained in the current document. In one implementation, a type of Bayesian optimization, discussed above, that incorporates the ADAM method, also discussed above, is used for the calibration step, with a total loss function defined by expression 3309 and expressions 3310. The total loss function employs loss function 3306 as well as the sum of squared deviations of model parameters from the mean of the parameters in the Gaussian prior.

Turning to FIG. 33B, parameter updates carried out by the ADAM optimizer are illustrated by expressions 3312. A regularization penalty is added to the loss function as indicated by expression 3314 to inhibit large-parameter-value results and the parameter values corresponding to physiological characteristics are constrained to fall within possible ranges 3316.

The optimized WBNM is generally validated using a variety of different methods. For example, as indicated by text 3318 in FIG. 33B, the simulated and observed signals can be compared in the time and frequency domains, phase coherence can be analyzed across multiple frequency domains, and functional conductivity patterns across brain regions may be analyzed. When MRI data is available, various of the model parameters can be initialized according to the MRI data, as indicated by text 3320 in FIG. 33B. This includes initializing coupling-strength parameters based on observed structural conductivity, initializing delay-time parameters based on observed distances between brain regions, initializing impulse-response parameters and other parameters based on cortical thickness and/or other structural properties of the brain anatomy, and initializing phase-coupling-strength parameters based on observed functional connectivity patterns. When MRI data is not available, parameter initialization may be based on head-scan and/or EEG data.

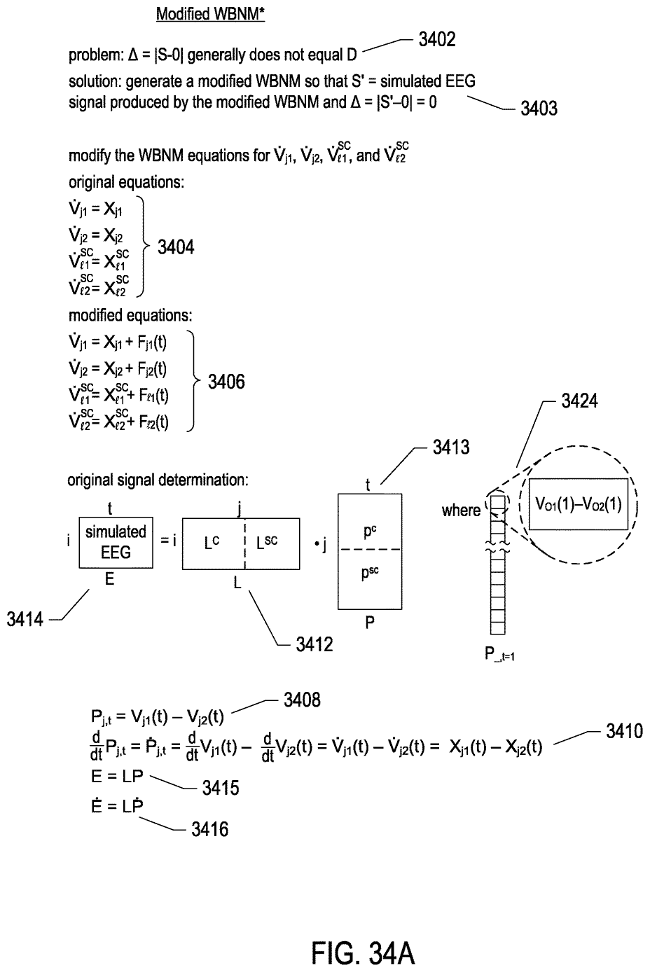

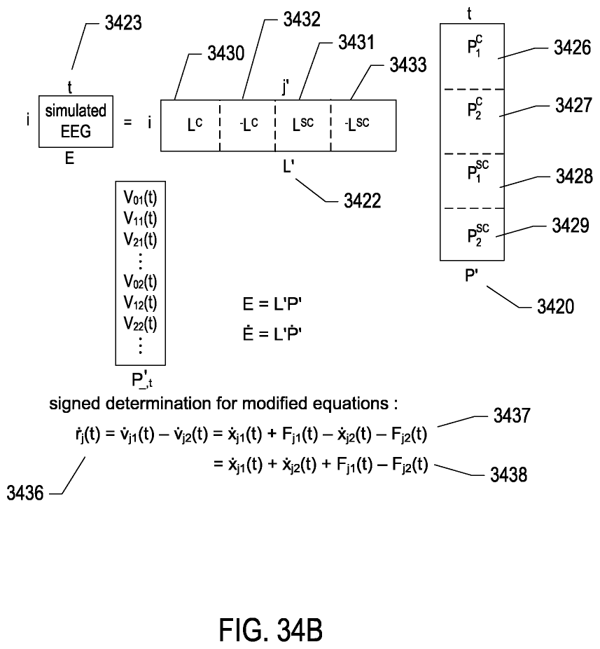

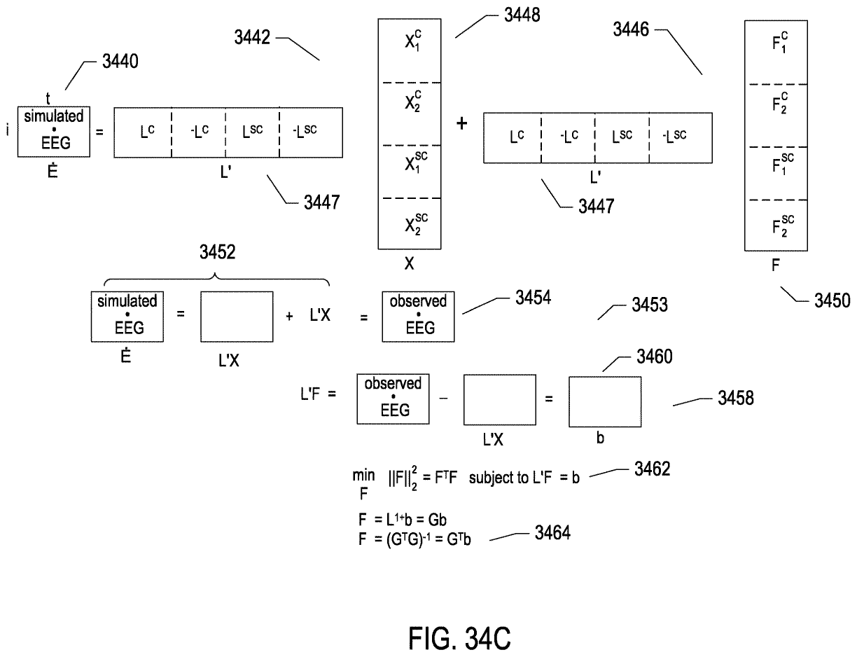

FIGS. 34A-C illustrate modification of the optimized or calibrated WBNM* to produce a more accurate modified model, mWBNM, that generates simulated EEG signals under a null stimulation that exactly match the patient's observed EEG under non-stimulating conditions. This is necessary in order to accurately compute severity levels and treatment efficiencies on which the currently disclosed treatment-planning and treatment-application methods depend, as discussed above with reference to FIGS. 27A-B. The model mWBNM provides an accurate baseline from which to measure the effects of control parameters, ensures that changes observed due to the application of stimuli reflect real effects rather than artifacts of model perfection, and facilitates reliable optimization of control parameters.

The problem addressed by modification of the optimized or calibrated WBNM* is that the difference between the simulated and observed EEG signals is non-zero 3402. Thus, modifications are made to the coupled differential equations on which the WBNM is based in order to force the model to exactly reproduce the patient's EEG signals 3403. In one implementation, the original expressions 3404 for the time differentials of the excitatory and inhibitory membrane potentials are modified to introduce adjustment terms, as shown in equations 3406, where the adjustment terms are denoted by the symbol “F” with the same double indexes as used for the interneuron-population membrane potentials of the cortical and subcortical regions. In the initial and calibrated WBNM, the membrane potentials of the cortical and subcortical regions are computed as the differences between the excitatory-interneuron membrane potentials and the inhibitory-interneuron membrane potentials 3408. Similarly, the time derivatives of the membrane potentials can be computed as the differences between the excitatory-interneuron-membrane-potential time derivatives and the inhibitory-interneuron-membrane-potential time derivatives 3410.

Using a horizontally partitioned lead-field matrix 3412 and a vertically partitioned local-membrane-potential matrix 3413, as shown at the top of FIG. 34B, the simulated EEG signals contained in a simulated-EEG matrix E 3414 is computed by multiplying the vertically partitioned local-membrane-potential matrix P from the left by the horizontally partitioned field matrix L. This corresponds to expression 3415 in FIG. 34A. The time differential of the simulated EEG can similarly be computed, as represented by expression 3416. An alternative approach is to use a modified local-membrane-potential matrix P′ 3420 and a modified field matrix L′ 3422 to compute the simulated EEG signals 3423. In this approach, the subtraction of the inhibitory membrane potentials from the excitatory potentials is not carried out prior to generating matrix P′, as in the case of matrix P (3424 in FIG. 34A), but is instead carried out during the multiplication of the modified matrix P′ by the modified matrix L′. Note that the modified matrix P is vertically partitioned into four partitions: (1) the excitatory-cortical-interneuron membrane potentials 3426; (2) the inhibitory-cortical-interneuron membrane potentials 3427; (3) the excitatory-subcortical-interneuron membrane potentials 3428; and (4) the inhibitory subcortical-interneuron membrane potentials 3429. The modified lead-field matrix L′ is correspondingly horizontally partitioned into four partitions including two positive partitions 3430-3431 and two negative partitions 3432 and 3433. Thus, while the modified matrices are multiplied, the resulting region membrane potentials are computed as the difference between the excitatory-neurons potentials and the inhibitory-neuron potentials.

The time derivative of the membrane potentials 3436 is computed according to expression 3437, with the right-hand side of expression 3437 rearranged to generate expression 3438. Unlike for the unmodified model, where the time derivative potentials are calculated as the difference between the two terms (3410 in FIG. 34A), the computation for the modified model involves four terms, two of which are the adjustment parameters introduced according to equations 3406 and 34A. The computations of the time differential of the simulated EEG signals 3440 in the modified model, as shown in FIG. 34C, is therefore carried out as the sum of two matrix multiplications 3442 and 3446. Both multiplications involve left multiplication by the L′ matrix 3447. The first multiplication involves a vertically partitioned matrix X 3448, corresponding to the two first terms of expression 3438 in FIG. 34B, and the second multiplication involves a vertically partitioned matrix F 3450, corresponding to the final two adjustment terms of expression 3438 in FIG. 34B. The computation of the time derivative of the simulated EEG signals is simplified as a first portion 3452 of equation 3453. In order to determine the values for the adjustment parameters, the time differential of the simulated EEG signals is set equal to the time differential of the observed EEG signals 3454. A rearrangement of the latter portion of the equation 3453 produces equation 3458, where the product of L′ and F, L′F, is alternatively represented by matrix b 3460. To find the values for the adjustment parameters, the squared adjustment parameters are minimized subject to the equivalence of matrix b and L′F, as indicated by expression 3462. This can generally be analytically solved using the Moore-Penrose inverse G of the modified lead-field matrix L′ as indicated in expression 3464. Other minimization techniques can be alternatively used to determine the values of the adjustment parameters when an inverse G cannot be found.

In the discussion of FIGS. 27A-B, which provide a control-flow diagram illustrating a general approach to treatment planning and stimulus application embodied by the currently disclosed methods and systems, the initial values for model control parameters are obtained by minimizing the severity level associated with simulated EEG data and new control-parameter values are selected for experimental stimulus application using treatment-efficacy values generated using severity levels computed from observed EEG signals. The final portion of the current document discusses how severity levels are computed from simulated and observed EEG signal data using a severity-level function implemented, in one implementation of the currently disclosed methods and systems, by a deep convolutional neural network. Note that the severity level may be computed for a specific neurological or psychiatric condition or may be a more generic indication of neurological health.

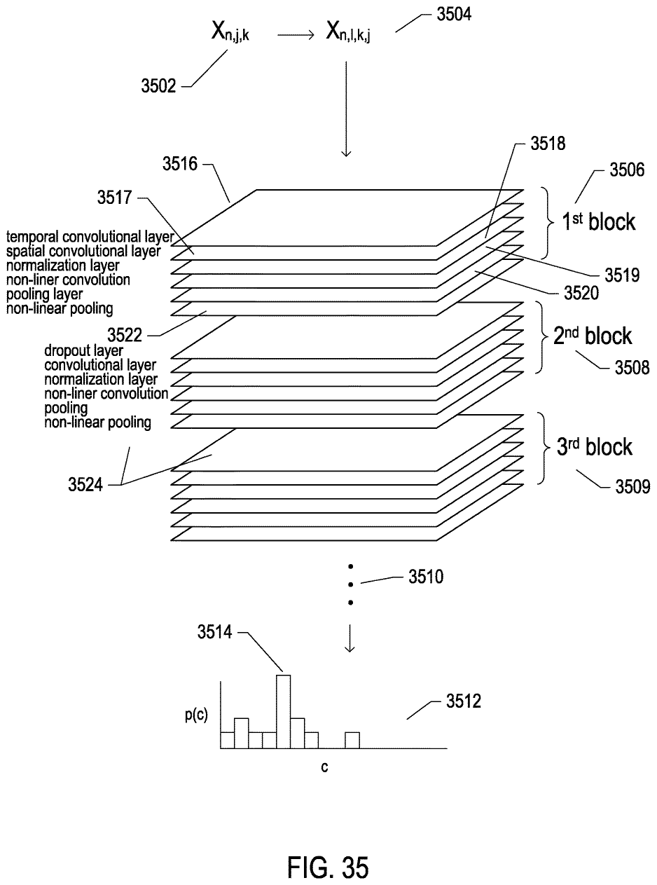

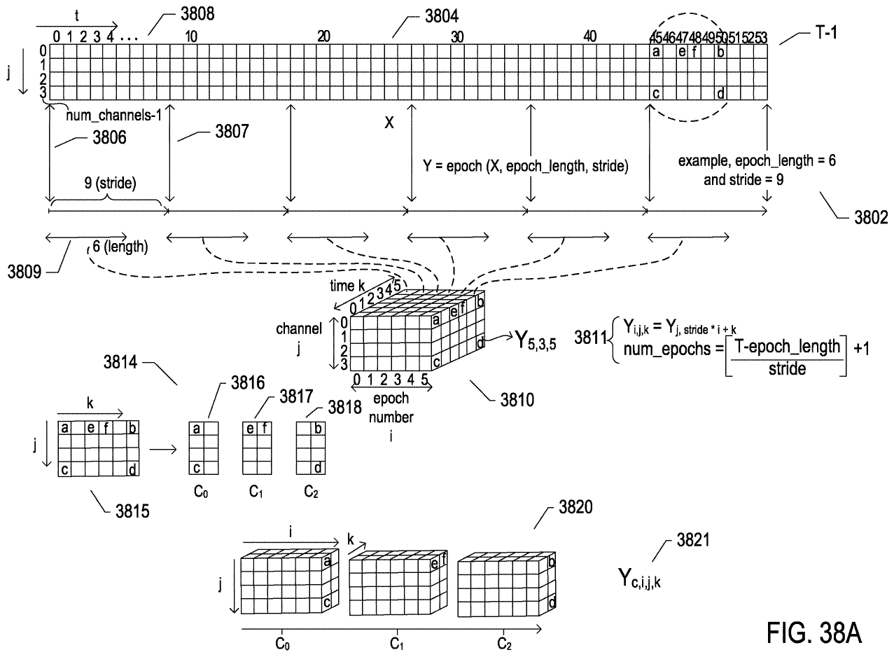

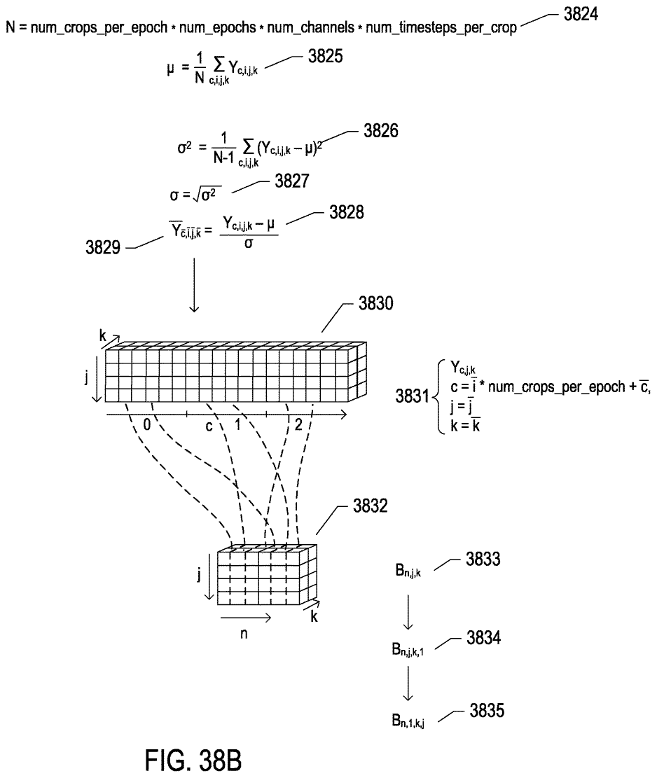

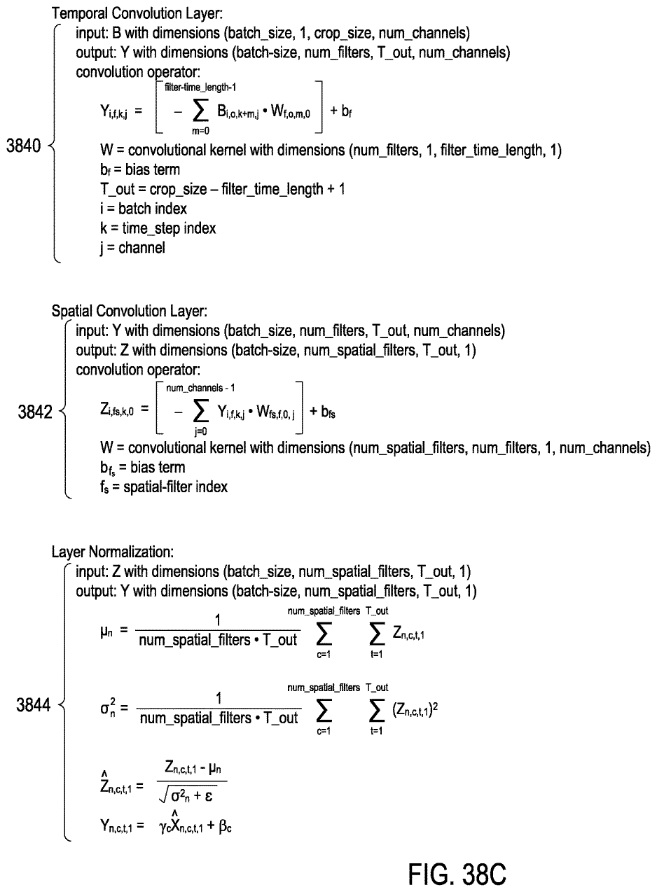

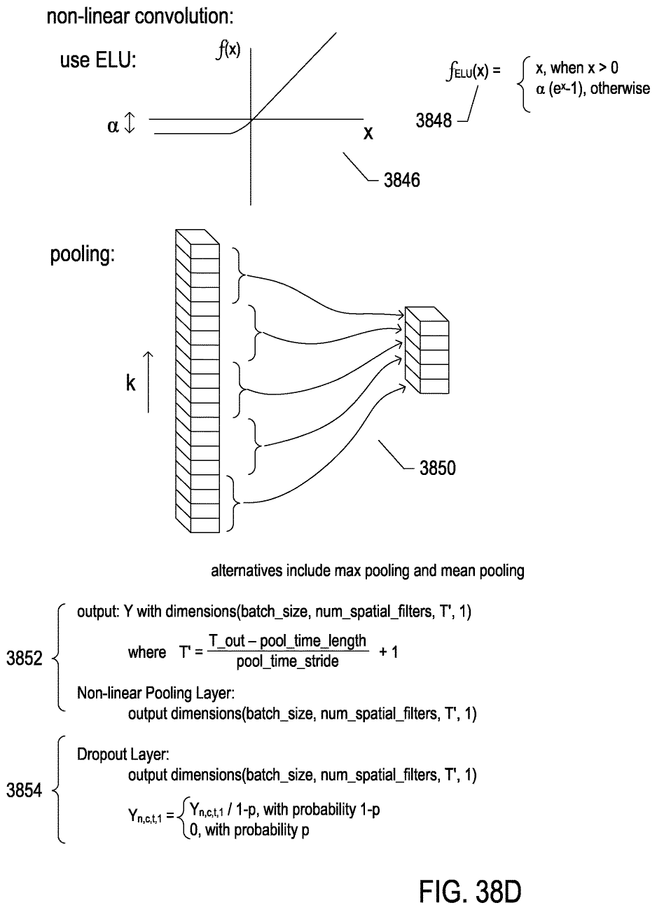

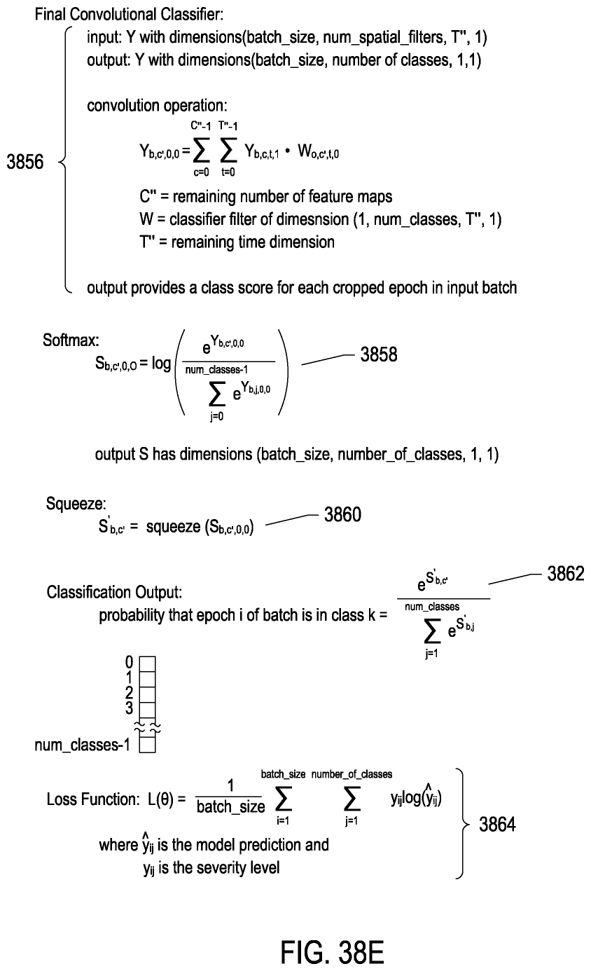

FIG. 35 illustrates one implementation of the convolutional neural network that implements the example severity-level function disclosed in the current document. A batch or sample 3502 prepared from EEG signal data is expanded to four dimensions 3504 by introducing a new singleton dimension as the second dimension (1 in indices n, 1, k, j of batch or sample X_{n,1,k,j}) so that certain already existing convolutional neural networks that expect 4-dimensional-tensor inputs can be employed. The convolutional neural network used in the described implementation includes a first block of layers 3506 and one or more additional blocks 3508-3509, with ellipsis 3510 indicating the possibility of additional blocks. The output of the convolutional neural network is a probability distribution 3512 that indicates the probabilities of the sample or batch having each of the various different possible categories. A category with greatest probability 3514 can be selected as the category associated with the sample or batch. The first block of layers includes a temporal convolution layer 3516, a spatial convolutional layer 3517, a normalization layer 3518, a non-linear convolution layer 3519, a pooling layer 3520, and a non-linear pooling layer 3522. Each successive block includes similar layers as well as a first dropout layer 3524. These layers are briefly described in FIGS. 38C-E.

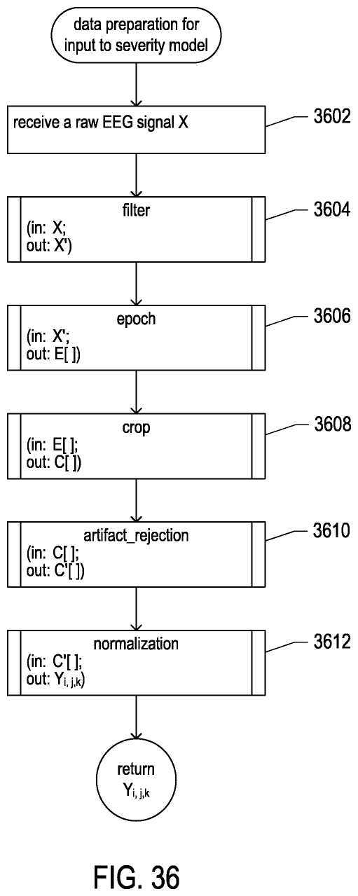

FIG. 36 shows a simple control-flow diagram illustrating the steps taken to prepare data for input to the convolutional neural network used to determine the severity level associated with simulated or observed EEG signals. In step 3602, the data-preparation routine receives raw EEG signals X, where X has the form of the EEG data matrix 1220 shown in FIG. 12B. In step 3604, the raw data is filtered to produce a filtered data stored in matrix X′. Filtering removes unwanted noise and artifacts from the raw data, including electrical interference from the environment and/or recording equipment, physiological noise from the patient's body unrelated to the electrical brain activity that is desired to be monitored, and artifacts related to a patient's body movements. As discussed below, bandpass filtering is often used as a component of the filtering process. In step 3606, the filtered data is processed to generate a sequence of relatively short segments, represented by an array E[ ] of short segments referred to as “epochs.” In step 3608, one or more smaller segments are extracted from each of the epochs to produce crops, stored in an array C[ ] of crops, via a process referred to as “cropping.” In a fourth step 3610, an additional artifact-removal process or processes are undertaken, implemented by techniques such as independent component analysis and/or template matching. In a fifth step 3612, the crop data is normalized to standardize the EEG signals across channels and time steps. The result of the data-preparation steps is a 3-dimensional tensor Y_{i,j,k} where index i refers to a particular crop, index j refers to a particular EEG channel, and index k refers to the crop size. When training the convolutional neural network, small subsets of the training data referred to as “batches” are used for training, as further discussed below.

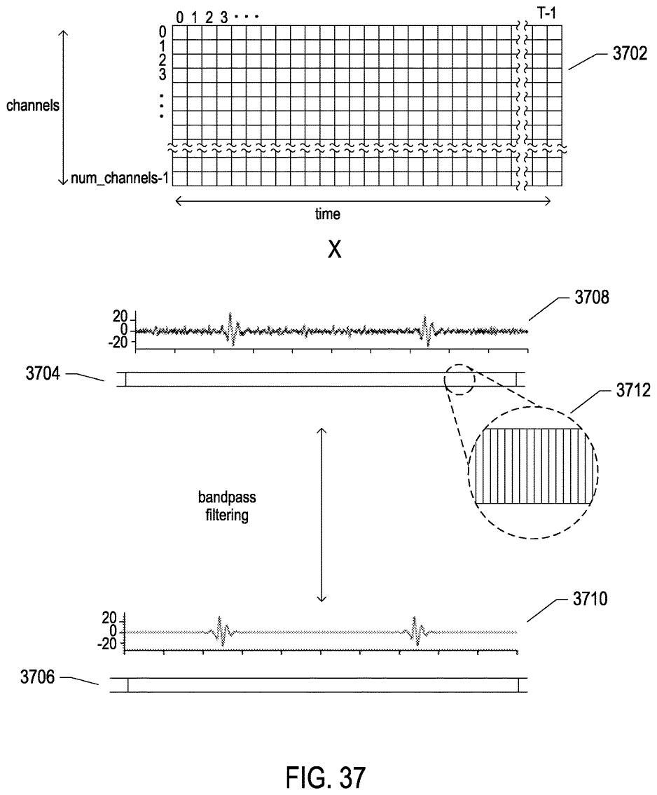

FIG. 37 illustrates bandpass filtering. The multi-channel-sensor signal is viewed as a 2-dimensional matrix 3702 in which each row represents a channel and each column represents a time point. Bandpass filtering is applied to each channel or signal component 3704 to produce a bandpass-filtered signal component 3706. In FIG. 37, the raw signal component is plotted in a 2-dimensional plot 3708 and the bandpass-filtered signal component is plotted in a 2-dimensional plot 3710 to illustrate the effects of bandpass filtering. Bandpass filtering can be carried out using convolution of Fourier transforms and by other means and selects a specific range of frequencies for the output bandpass-filtered signal. As indicated by inset 3712, a signal component may include many time points separated by very short time intervals to provide sufficient resolution.

FIGS. 38A-E illustrate severity-level determination. FIGS. 38A-B illustrate additional processing steps, mentioned above with reference to FIG. 36, used to generate signal samples and batches. As shown in diagram 3802 at the top of FIG. 38A, a filtered EEG signal 3804 is partitioned into multiple contiguous partitions, each partition indicated by a pair of double-headed arrows, such as double-headed arrows 3806-3807 that together identify the first partition 3808 consisting of 9 time points. The partitioning is carried out using a fixed stride equal to 9. Epochs of length 6 are selected from each partition, where the epochs are indicated by horizontal double-headed arrows, such as double-headed arrow 3809 indicating the first epoch in the first partition 3808. The epochs are then assembled into the tensor Y_{i,j,k} 3810, where index i refers to a particular epoch, index j refers to a particular EEG channel, and index k refers to an offset within an epoch. Expressions 3811 indicate the relationship between the tensor and the filtered EEG signal and a computation of the number of epochs in the tensor based on the number of time points in the filtered EEG signal, the length of the epochs, and the stride. Of course, the very short stride and epoch length facilitate illustration, but longer strides in length would generally be used. Diagram 3814 illustrates partitioning of an epoch 3815 into three crops 3816-3818. Each crop includes data from two successive time points extracted from the epoch. Diagram 3820 shows cropping of tensor 3810 to produce the 4-dimensional tensor Y_{c,i,j,k} 3821, where the initial index c refers to the cluster number within the epoch indexed by the second index i.

Turning to FIG. 38B, the total number of data points N within the tensor Y_{c,i,j,k} 3821 is computed according to expression 3024. The mean data-point value is computed according to expression 3825, the variance for the N data points is estimated according to expression 3826, and the standard deviation is obtained from the variance according to expression 3827. Normalization of the data values within the tensor Y_{c,i,j,k} 3821 is then carried out according to expression 3828. The 4-dimensional tensor obtained by normalization 3829 can be compressed into a 3-dimensional tensor Y_{c,j,k} 3830 according to expressions 3831. A 3-dimensional batch tensor 3832, symbolically represented as B_{n,j,k} 3833, where index n refers to a particular batch, is generated by selecting the data values for one or more time points from each cluster to form each batch. The batch tensor is expanded to add a fourth dimension with a single element 3834 in order to produce a tensor with the number of dimensions needed for certain third-party processing routines, and the dimensions are shuffled to produce the 4-dimensional batch tensor 3835 that contains data in a form that can be input to the convolutional neural network, described below.

FIGS. 38C-E illustrate the layers of the convolutional neural network introduced with reference to FIG. 35. The temporal convolution layer (3516 in block 2506 in FIG. 35 and in additional blocks) captures the local temporal patterns in the input EEG data by applying a series of filters to the data, where each filter is designed to detect specific temporal features and is applied via a convolutional operator. This layer helps the convolutional neural network to learn temporal relationships. The temporal convolution layer is described by expressions 3840 in FIG. 38C. The spatial convolution layer (3517 in block 2506 in FIG. 35 and in additional blocks) applies a number of spatial filters to the output from the temporal convolution layer. The spatial convolution layer is described by expressions 3842 in FIG. 38C. The batch-normalization layer (3518 in block 2506 in FIG. 35 and in additional blocks) carries out normalization via a computed batch mean and variance. The batch normalization-layer is described by expressions 3844 in FIG. 38C.

Turning to FIG. 38D, the non-linear convolution layer (3519 in block 2506 in FIG. 35 and in additional blocks) employs the ELU activation function plotted in plot 3846 and defined in expression 3848. The pooling layers (3520-3522 in block 2506 in FIG. 35 and in additional blocks) compress a sample or batch along the temporal or time-step dimension, as indicated by diagram 3850. The value used to represent a set of contiguous data points in the time-step dimension may be the maximum value of a data point in the set of contiguous data points, a mean value of the data points in the set of continuous data points, or another type of computed value. The pooling layers are described by expressions 3852 in FIG. 38 D. The dropout layer in each of the second through final blocks of the convolutional neural network randomly sets various data points to 0 and is used only during training. The dropout layer is described by expressions 3854 in FIG. 38D. Turning to FIG. 38E, the convolutional neural network includes a final convolutional layer, or classifier layer, described by expressions 3856. The classifier layer generates a value for each different class using the log-softmax function, as indicated by expression 3858. A squeeze operation compresses output S of the log-softmax function to a 2-dimensional tensor 3860. As indicated by expression 3862, these values are used to generate a probability distribution (plot 3512 in FIG. 35) that is used to assign a severity level, or severity class, to a sample or batch. Finally, a cross-entropy loss function 3564 is used during training of the convolutional network.

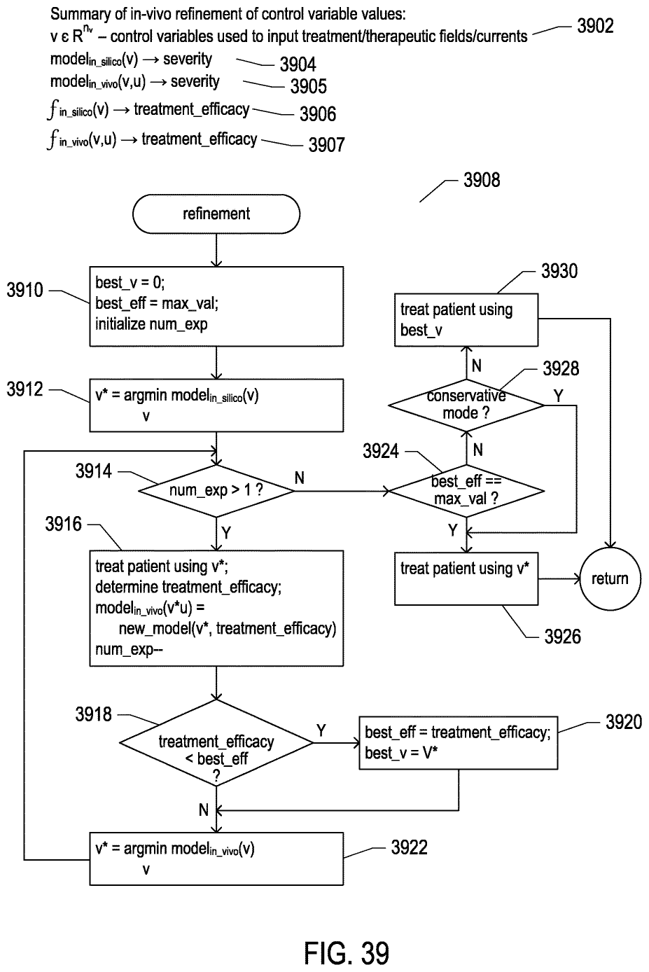

A general control-flow diagram for treatment planning and application is discussed above with reference to FIGS. 27A-B. FIG. 39 provides a control-flow diagram for a routine “refinement” that represents an alternate description of steps 2718-2732 in FIGS. 27A-B, which show the in vivo refinement of control variables used for treatment application via limited experimentation. The symbol v used in FIG. 39 represents the n control variables for application of treatments to a patient, as indicated by expression 3902. Variable v is thus a vector variable, as is variable u, introduced below. Each control variable can be considered to have a floating-point or integer value. There are two models, both comprising a modified WBNM together with a severity-level function: (1) model_{in_silico} 3904, a computational model that takes control-variable values v as an argument, that is based on a modified WBNM, and that computes a severity-level indication used to return a treatment efficacy; and (2) model_{in_vivo} 3905, a computational model that takes both control-variable values v and hidden-variable values u as arguments, that is based on a modified WBNM, and that computes a severity-level indication used to return a treatment efficacy. The variable u represents unobservable patient-specific information acquired via experimentation. The function ƒ_{in_silico}(v) returns the treatment-efficacy value returned by model_{in_silico}. The function ƒ_{in_silico}(v) does not consider experimental results but is based on the change in severity levels between a reference severity level and the severity level determined for the simulated EEG signal generated after application of a simulated therapeutic stimulus. By contrast, the function ƒ_{in_vivo}(v, u) is modified after each experimental application of a therapeutic stimulus to produce an estimated severity level in conformance with the treatment efficacies observed after each experiment. As discussed below, in detail, this is accomplished by evolving a transform applied to the control-variable values v. Thus, model_{in_vivo} differs from model_{in_silico} in using ƒ_{in_vivo}(v, u) rather than ƒ_{in_silico}(v) to determine a predicted treatment efficacy for a set of control-variable values.

The control-flow diagram for the routine “refinement” 3908 is shown in the lower portion of FIG. 39. In step 3910, local variable best v is set to 0, local variable best_eff is set to some large value max_val and local variable num_exp is set to the maximum allowable number of therapy applications. In step 3912, a next set of control variables v* is obtained by optimizing the control variables with respect to the severity level produced by model_{in_silico}. When the value of local variable num_exp is greater than 1, as determined in step 3914, then, in step 3916, a therapeutic treatment is applied using control variables v*, the efficacy of the treatment is determined, a new model_{in_vivo} is generated from v* and the determined treatment efficacy by updating ƒ_{in_vivo}, as discussed below, and local variable num_exp is incremented. When the observed treatment efficacy is less than the value stored in local variable best_eff as determined in step 3918, best_eff is set to the observed treatment efficacy and best v is set to v*, in step 3920. In step 3922, new control variables are generated using the new model_{in_vivo} generated in step 3916. Application of treatments and generation of a new model_{in_vivo} continue until the value stored in num_exp falls to 1, as detected in step 3914, leading to flow of control to step 3924, where it is determined whether or not local variable best_eff still has the initial value max_val. If so, there has been no experimentation, and the control variables determined in step 3912 are used to treat the patient in step 3926. Otherwise, in step 3928, the routine determines whether or not it is operating in conservative mode. If so, the final set of control variables determined in step 3922 is used to treat the patient in step 3926. Otherwise, the control variables stored in local variable best v are used to treat the patient in step 3930.

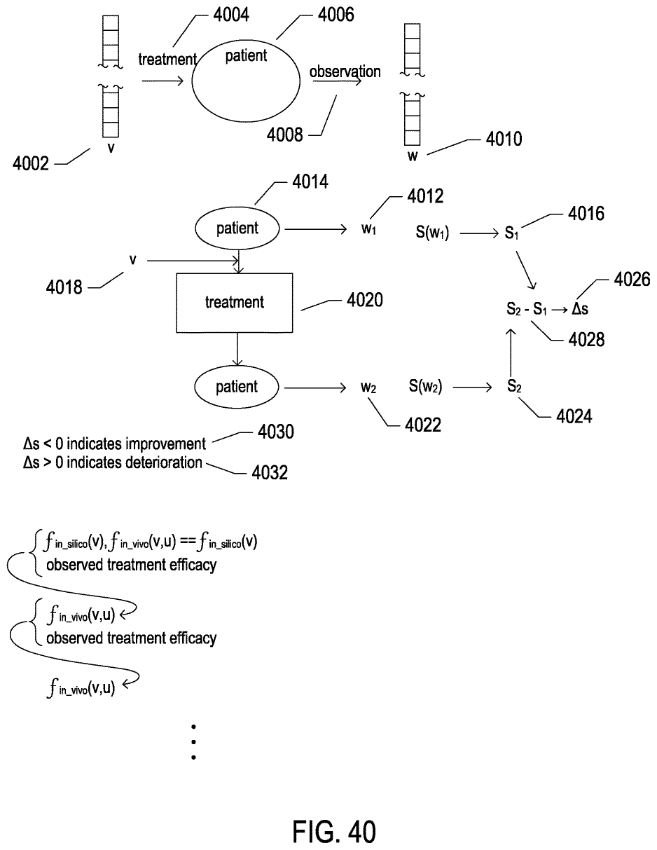

FIG. 40 illustrates determination of the change in severity level following therapeutic treatment. Treatment comprises using a set of control variables 4002 to control application of a therapeutic stimulus 4004 to a patient 4006 and then observing 4008 a patient response 4010. In examples provided in this document, the patient response generally consists of EEG-signal data. In order to determine the change in severity level resulting from treatment, EEG-signal data 4012 is obtained from the patient 4014 prior to treatment and used to determine an initial severity level 4016. The control variables 4018 are then used to apply treatment 4020 to the patient after which EEG-signal data is again obtained from the patient 4022. The after-treatment EEG-signal data is used to generate a second severity value 4024. The change in severity level ΔS 4026 is obtained as a difference between the first severity level and the second severity level 4028. When the change in severity is less than 0, improvement is indicated 4030. When the change in severity level is greater than 0, deterioration of the patient is indicated 4032. The treatment efficacy is a value computed from the observed change in severity level ΔS. In the currently disclosed implementation, the lower the treatment-efficacy value, the more effective the treatment. The computation of the treatment-efficacy value may involve linear or non-linear scaling, as the change in severity level ΔS may vary with varying pre-treatment severity levels.

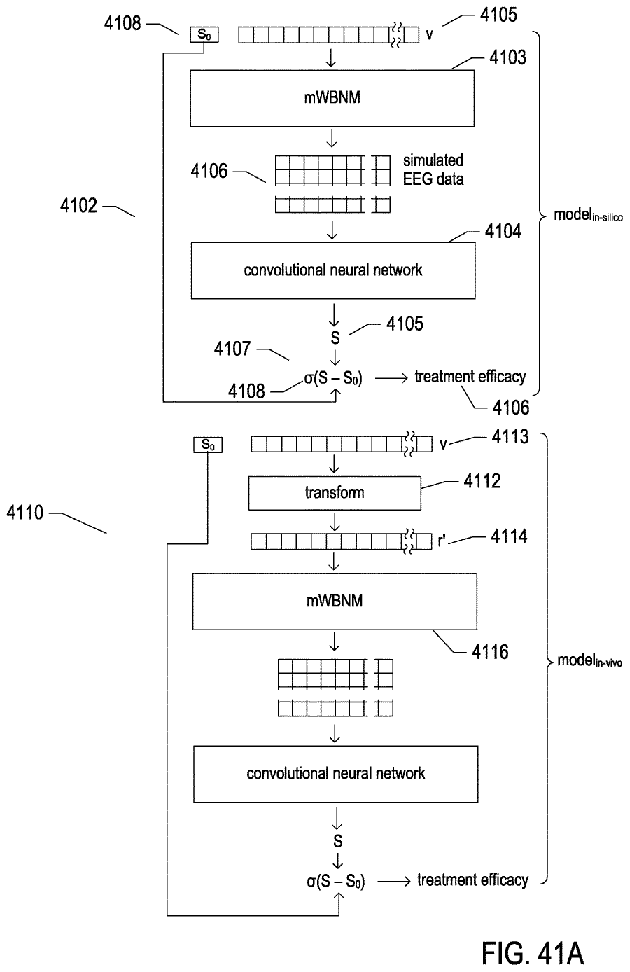

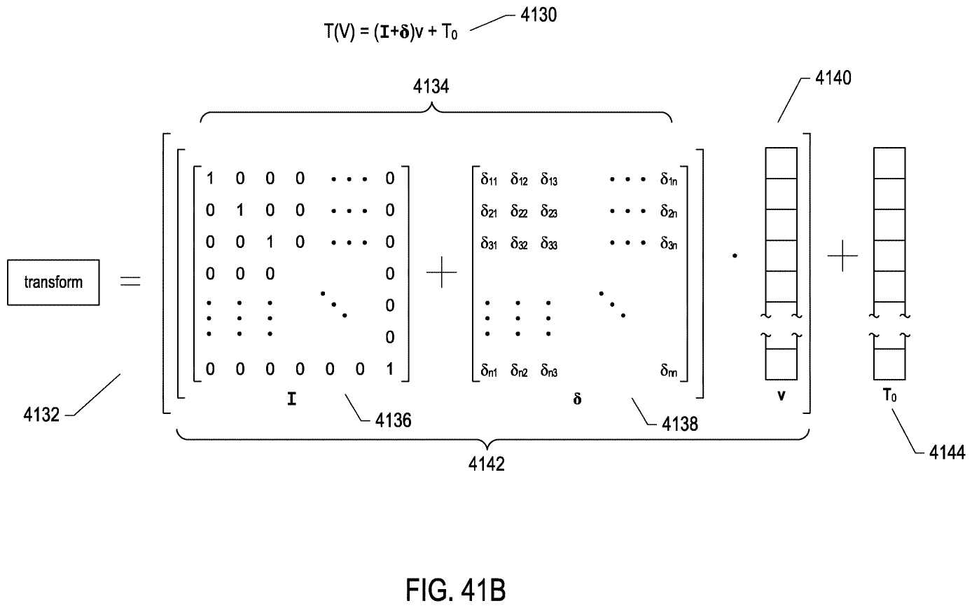

FIGS. 41A-B illustrate one implementation of model_{in_silico} and model_{in_vivo}. An implementation of model_{in_silico} 4102 is shown at the top of FIG. 41A containing a modified WBNM, mWBNM 4103 and a severity-level-determining convolutional neural network 4104. A vector of control-variable values v 4105 is input to the mWBNM, which outputs simulated EEG data 4106 representing the predicted EEG data that would be observed following treatment of the patient controlled by the control-variable values v. The convolutional neural network outputs a probability distribution from which a predicted severity 4105 is selected as the most likely severity level corresponding to the simulated EEG data. Finally, the predicted treatment efficacy 4106 of the treatment applied according to the control-variable values contained in the vector v is computed 4107 using the output from the convolutional neural network as well as a reference severity level 4108. In this example, computation of the treatment efficacy involves the product of a difference between the predicted severity level and a scaling function σ 4108. There are numerous different methods and functional forms that can be used for computing the treatment efficacy in various different implementations. The actual value of the treatment efficacy is less important than the relative values of treatment-efficacy values computed for different treatments, since it is the relative values that control optimization of the control-variable values, as discussed above with reference to FIG. 39.

An implementation of model_{in_vivo} 4110 is shown in the lower portion of FIG. 41A. The implementation of model_{in_vivo} differs from the implementation of model_{in_silico} only in the inclusion of a transform function 4112 that transforms input control-variable values v 4113 to transform to control-variable values v′ 4114 which are input to the modified WBNM, mWBNM, 4116. The initial model_{in_vivo}, at the beginning of treatment experimentation in step 3910 of the control-flow diagram shown in FIG. 39, is identical to model_{in_silico}, with the initial transform function essentially performing a null transform since no additional information has been obtained through limited experimentation. However, with each therapeutic-treatment experiment, in the loop of steps 3914, 3916, 3918, 3920, and 3922 in FIG. 39, model_{in_vivo} is modified by modifying the transform function 4112 to reflect the accumulated additional patient information obtained through limited experimentation.

FIG. 41B illustrates one implementation of the transform (4112 in FIG. 41A) used in the model_{in_vivo}. A matrix expression 4130 for the transform is shown at the top of FIG. 41B. The transform is shown diagrammatically in the middle portion 4132 of FIG. 41B. A matrix 4134, obtained by adding the identity matrix 4136 to a deformation matrix 4138, multiplies a control-variable vector 4140 to produce a resultant transformed vector 4142. A constant transformation vector 4144 is added to the resultant transformed vector to produce the final modified control-variable vector (4114 in FIG. 41A).

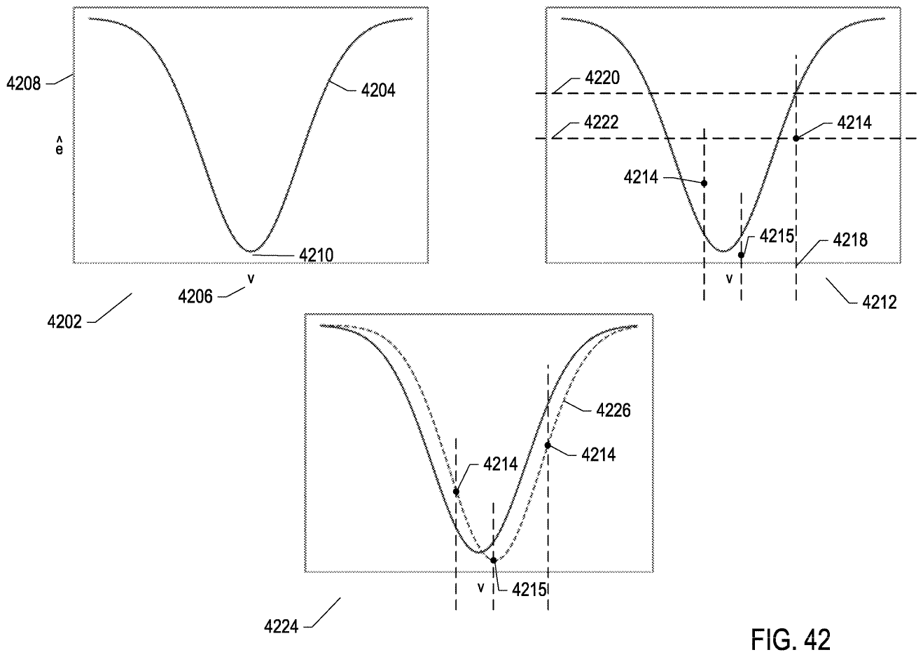

FIG. 42 illustrates the deformation or modification of a model to produce a model that incorporates additional patient information gleaned from limited treatment experimentation. In FIG. 42, the symbol “ê” denotes a predicted treatment efficacy and the symbol “eₒ” denotes an observed treatment efficacy. A first plot 4202 shows the ê-vs-v curve 4204 for control-variable values plotted with respect to a horizontal axis 4206, with the efficacy estimate plotted with respect to a vertical axis 4208. Note that values of the vector v are plotted along a one-dimensional horizontal axis for illustration convenience. Vector values would need to be plotted in a high-dimensional space, but that is not possible for vectors with more than 3 elements. The ê-vs-v curve 4204 corresponds to the model prior to a next modification to update the model in view of additional experimentally derived information. In this simple example, the optimal value for the control variables lies at the bottom 4210 of the well-shaped ê-vs-v curve. In a second plot 4212, three experimentally derived data points 4214-4216 are plotted along with the generic ê-vs-v curve. In other words, for example, for control-variable values v represented by point 4218 on the horizontal axis, the model estimates an efficacy of 4220 but an experimental treatment or therapy corresponding to the control-variable value of 4218 produces a different observed efficacy 4222. The transform discussed above with reference to FIG. 41B is then used, as illustrated in plot 4224, to shift, deform, and align the ê-vs-v curve 4226 with the experimentally derived data points 4214-4216. Thus, the transformation of the control variables produces a slightly modified or deformed model that retains much of the information contained in the original model from which it is produced. Simply trying to fit an arbitrary curve through a handful of experimentally derived data points, without the benefit of original model, would not be possible or, perhaps stated more accurately, would not sufficiently constrain the form of the curve produced by the model for the model to accurately predict treatment efficacies over a reasonable range of possible control-variable vectors. A series of deformations retains a great deal of knowledge accumulated over many treatments of many different patients encompassed in model_{in_silico} while adjusting model_{in_vivo} to accurately predict treatment efficacies.

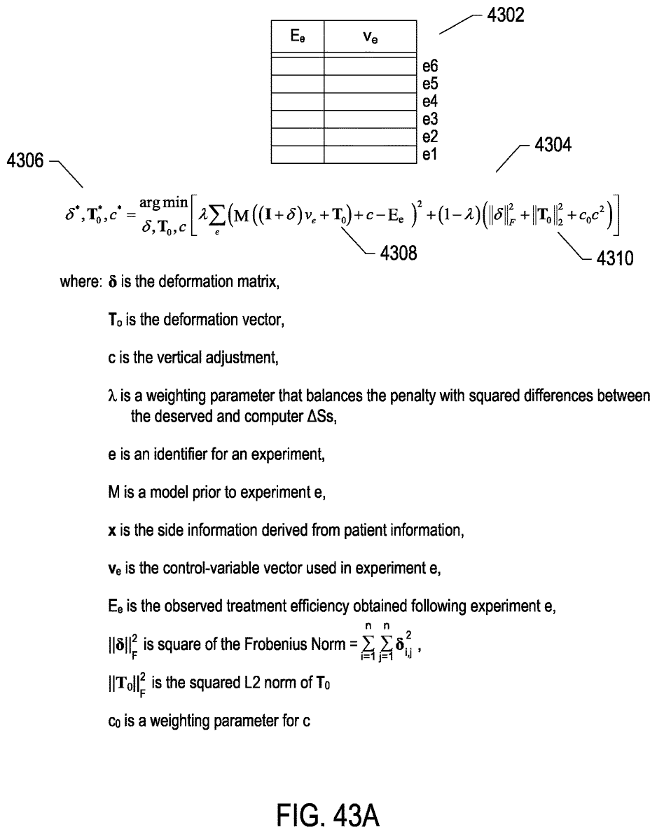

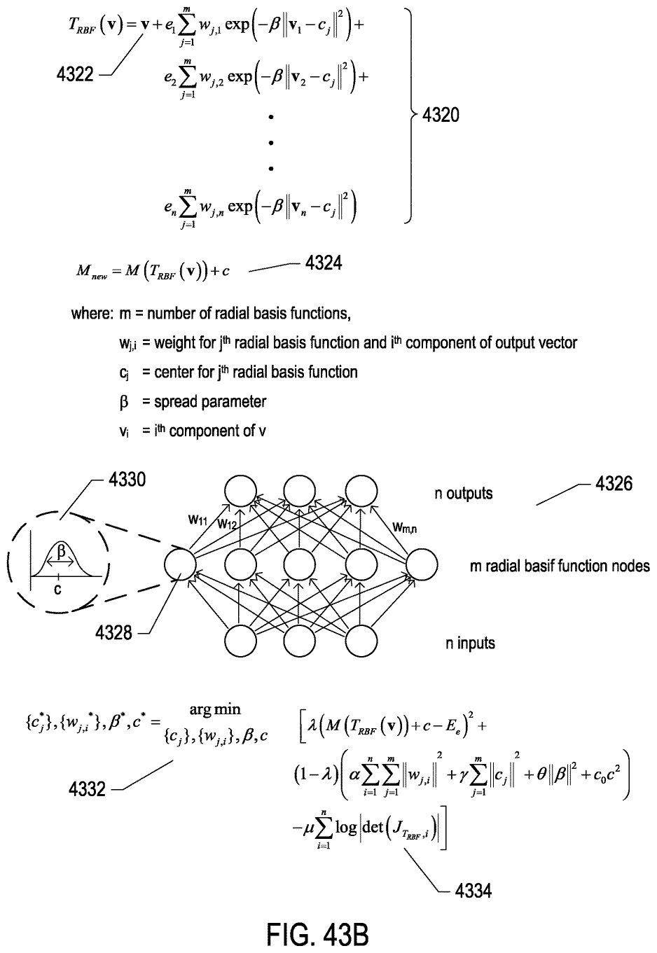

FIGS. 43A-B illustrate the deformation process introduced above with reference to FIG. 42. The process is illustrated in FIG. 43A. Table 4302 contains the results from multiple therapeutic-treatment experiments, with “Eₑ” representing the treatment efficacy observed for experiment e and “vₑ” representing the control-variable values used to apply the treatment. The deformation process minimizes the bracketed value 4304 over possible values of the deformation matrix δ, transformation vector T₀, and vertical-alignment constant c, as indicated in expression 4306. A first term 4308 in the bracketed expression 4304 is the sum of the squared differences between the estimated efficacies of a model M parameterized, in part, by particular values of the deformation matrix δ, transformation vector T₀, and vertical-alignment constant c and the experimentally observed efficacies and the second term 4310 is a penalty term that penalizes large-magnitude deformation matrices δ, transformation vectors T₀, and vertical-alignment constants c. Thus, the minimization of the value represented by the bracketed expression conceptually represents a search for an optimal deformation matrix δ*, transformation vector T₀*, and vertical-alignment constant c* that minimizes the sum of the squared differences between the efficacy estimates generated by the model parameterized, in part, by the optimal deformation matrix δ*, transformation vector T₀*, and vertical-alignment constant c* and the experimentally determined efficacies while, at the same time, constraining the optimal deformation matrix δ*, transformation vector T₀*, and vertical-alignment constant c* by using the penalty term to avoid larger-than-desirable changes to the generic efficacy-estimation function. The penalty term increases in magnitude with increase in the magnitudes of the deformation matrix δ*, transformation vector T₀*, and vertical-alignment constant c* to penalize larger deformations. This penalty-term-constrained minimization seeks an accurate new model that does not differ too greatly from a preceding model. Any of many standard constrained optimization/minimization techniques can be employed to generate the new model from a table of experimentally derived Eₑ/v pairs and an existing model.

FIG. 43B illustrates an alternative transformation of the input vector for deformation of a generic efficacy-estimation function to that discussed above with reference to FIGS. 41A-B. The alternative transformation uses a radial-basis-function-network transformation described by expression 4320 at the top of FIG. 43B. In this expression, the values of the components of the input vector 4322 are altered by the addition of values computed from the radial-basis-function network, with e₁, e₂, . . . , eₙ representing the orthonormal basis vectors of control-variable vectors. Expression 4324 represents the transformation of a model to a new model in similar fashion to expression 4130 in FIG. 41B. The radial-basis-function network can be viewed as a neural network 4326 with each hidden node, such as hidden node 4328 representing a radial basis function 4330 with a specific center c and spread p. Gaussian-like functions are commonly used as radial-basis functions. Determination of the new model is also a constrained optimization/minimization, as indicated by expression 4332 in FIG. 43B, as is the case for the constrained optimization/minimization discussed above with reference to FIG. 43A. In certain implementations, an additional penalty term 4334 is included in the bracketed expression for the value that is minimized. This additional penalty term attempts to force the patient-specific efficacy-estimation function towards continuous differentiability.

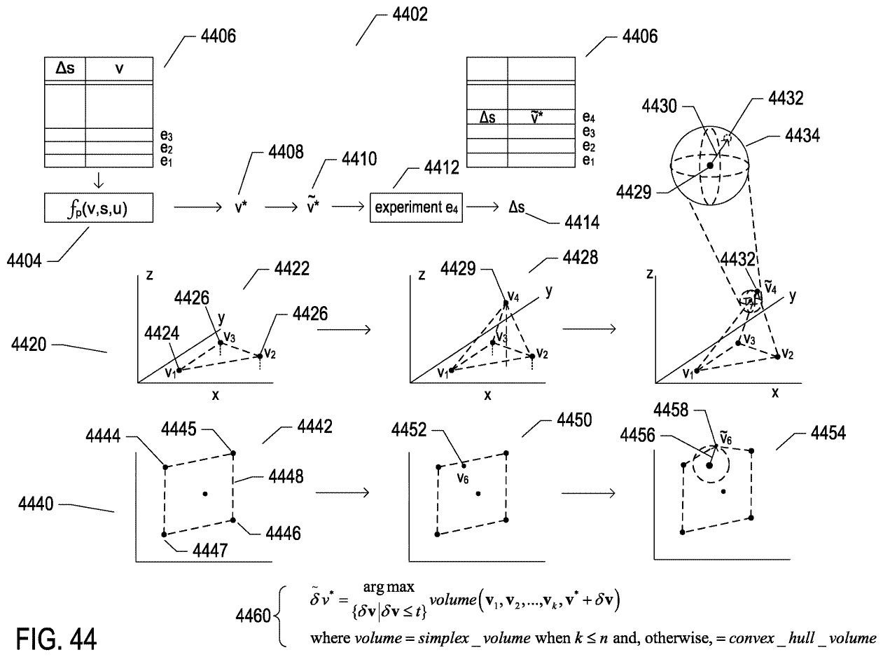

FIG. 44 illustrates a technique used, in certain implementations of the currently disclosed methods and systems, to expand the search space of control-variable vectors explored in the constrained optimization/minimization processes discussed above with reference to FIGS. 43A-B. This process is illustrated in a first diagram 4402 the top of FIG. 44. As discussed above, a constrained optimization/minimization process is used to generate a new model 4404 from a table of experimentally derived Eₑ/vₑ pairs 4406. The new model is then used to generate a new control-variable vector 4408, or treatment plan, for a next experiment. Rather than use this treatment plan, the search-space expansion technique modifies the new treatment plan to create a modified treatment plan 4410 that is then used in a next experiment 4412 to generate a new observed result 4414 which is added to the table of Eₑ/vₑ values 4406 along with the modified treatment plan 4414. The generation of the modified control-vector is illustrated in two sets of diagrams 4420 and 4440 in a middle and lower portion of FIG. 44, respectively. In 3-dimensional plot 4422, points 4424-4426 represent the current control vectors in the table of Eₑ/vₑ values 4406. Plot 4428 illustrates addition of a next control-variable vector 4429 to the collection of control-variable vectors stored in the table, with control-variable vectors represented by points in a 3-dimensional space, implying that the control-variable vectors each have three elements. However, in order to expand the search space, rather than adding the new control-variable vector 4429, a small displacement vector 4430 is generated and added to the initial next control-variable vector 4429 to produce the modified control-variable vector 4432 which is added to the table of Eₑ/vₑ values instead of the initial next control-variable vector 4429. In the case that the number of control-variable vectors in the table, including the newly added control-variable vector, can be viewed as representing the vertices of a simplex, such as a triangle or tetrahedron in a 3-dimensional or lower-dimensional space, the displacement vector 4430 is determined as a displacement vector, equal to or less than a fixed radius of a sphere 4434, that generates the greatest resulting area or volume for the simplex. In 2-dimensional plot 4442, five 2-dimensional control-variable vectors 4444-4447 have already been entered into the table of Eₑ/vₑ values. Dashed rectangle 4448 represents the 2-dimensional convex hull of these 5 points. As shown in plot 4450, a next control-variable vector to be added to the table represented by point 4452 falls within the convex hull. However, as shown in plot 4454, a displacement vector 4456 can be generated for the new control-variable vector to modify the new control-variable vector 4458 such that the convex hull is expanded in area. Thus, generating a modified next control-variable vector is a constrained optimization/maximization process as indicated by expressions 4460 at the bottom of FIG. 44, which is valid for a control-variable-vector of any dimension.

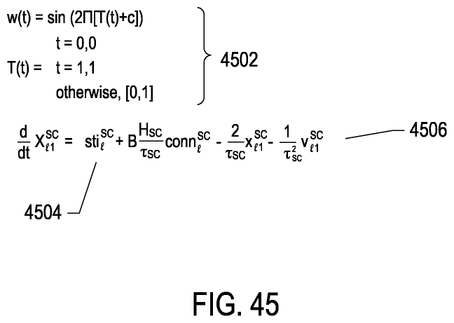

FIG. 45 illustrates a few modifications to the WBNM that can be used to adapt a WBNM to other therapeutic methods. Many of the other therapeutic methods involve waveform choices which are difficult to determine or select. A deformation-based method that expresses a waveform as a continuous deformation of the sine wave is indicated by expressions 4502 in FIG. 45, where T(t) is a transformation. A WBNM can be adapted for therapeutic treatments that employ implanted neuromodulation devices, such as deep-brain stimulation (“DBS”). In one method, for subcortical regions, a new term 4504 is added to the differential of equations for the subcortical regions, such as equation 4506, to represent external stimulation applied to subcortical regions. Many other such modifications can be used to adapt a WBNM for various different alternative types of therapeutic treatment.

The present invention has been described in terms of particular embodiments, but it is not intended that the invention be limited to these embodiments. Modifications within the spirit of the invention will be apparent to those skilled in the art. For example, any of many different implementations of the currently disclosed methods and systems can be obtained by varying various design and implementation parameters, including modular organization, control structures, data structures, and other such design and implementation parameters. For example, the number of different machine-learning techniques can be used to generate a severity level from simulated and observed EEG-signal data. As another example, a variety of different coupled-differential-equation models can be used to simulate electrical activity within the human brain resulting from input signals. Stimulation therapies may include transcranial direct current stimulation (“tDCS”), transcranial alternating current stimulation (“tACS”), transcranial focused ultrasound stimulation (“tFUS”), transcranial photobiomodulation (“tPBM”), transcranial magnetic stimulation (“TMS”), and vagus nerve stimulation (“VNS”).

1. A system that generates a treatment plan for treating a patient, the system comprising:
a first device or system that records data from the patient;
a second device or system that applies a treatment to the patient according to a set of control parameters;
one or more computer systems; and
a treatment-planning method, implemented as computer instructions stored and executed on one or more of the one or more computer systems, that
receives patient data and treatment information,
uses electrical-activity data included in the received patient data and/or recorded by the first device or system to generate a whole-brain-network model (“WBNM”), based on a computational model, that simulates electrical activity in the patient's brain in response to input signals, and outputs a treatment-efficacy value corresponding to the simulated electrical activity,
generates an optimized WBNM (“WBNM*”) by adjusting model parameters of the WBNM to minimize differences between the simulated electrical activity generated by the WBNM* and the electrical-activity data,
modifies the WBNM* to generate a modified WBNM (“mWBNM”) by modifying the computational model to ensure that the mWBNM generates simulated electrical activity that matches the electrical-activity data,
uses the mWBNM to generate a treatment plan for the patient, and
when limited experimentation is indicated in the received therapeutic-treatment information, carries out up to a specified number of experiments, each experiment carried out by
using a most recently generated treatment plan to control the second device or system to apply a treatment to the patient,
using the first device or system to record electrical-activity data, from which a treatment efficacy is determined,
using the determined treatment efficacy to adjust the mWBNM, and
using the adjusted mWBNM to generate a next treatment plan for the patient.

2. The system of claim 1 wherein the patient is treated for one or more neurological and/or psychiatric disorders and conditions.

3. The system of claim 1 wherein the second device or system stimulates electrical activity within the patient's brain.

4. The system of claim 3 wherein the second device or system applies one or more of:
transcranial direct current stimulation (“tDCS”);
transcranial alternating current stimulation (“tACS”);
transcranial focused ultrasound stimulation (“tFUS”);
transcranial photobiomodulation (“tPBM”);
transcranial magnetic stimulation (“TMS”); and
vagus nerve stimulation (“VNS”).

5. The system of claim 1 wherein the first device or system is an electroencephalography (“EEG”) device or system that records multi-channel signals.

6. The system of claim 5 wherein the patient data and treatment information received by the method includes one or more of:
EEG data;
patient information, including age, one or more diagnoses, health history, treatment parameters, including the maximum number of experimental procedures that can be performed on the patient to refine treatment control parameters; and
MRI data that can facilitate generating the WBNM.

7. The system of claim 6 wherein the WBNM includes:
the computational model which is solved to generate one or more functions that output membrane potentials for cortical and subcortical regions of the brain at a point in time resulting from specified input signals;
a simulator that generates simulated EEG data from membrane potentials output by the one or more functions for successive time points;
a severity-level function that outputs a severity-level indication corresponding to input simulated EEG data generated by the simulator; and
a treatment-efficacy component that outputs a treatment-efficacy value corresponding to the severity-level indication output by the severity-level function.

8. The system of claim 7 wherein the computational model of the WBNM includes:
a model portion for cortical regions that each includes
a population of pyramidal cells,
a population of excitatory interneurons, and
a population of inhibitory interneurons;
a model portion for subcortical regions that each includes
a population of excitatory interneurons, and
a population of inhibitory interneurons;
synapses that connect the pyramidal cells in a cortical region to excitatory interneurons of the same region;
synapses that connect the pyramidal cells in a cortical region to inhibitory interneurons of the same region;
synapses that connect the excitatory interneurons in a cortical region to pyramidal cells of the same region;
synapses that connect the inhibitory interneurons in a cortical region to pyramidal cells of the same region;
synapses that connect cortical regions to one or more other cortical regions and to one or more subcortical regions; and
input of external stimuli to cortical regions.

9. The system of claim 8 wherein each pyramidal-cell and interneuron population is represented by an impulse response function and a sigmoidal gain function.

10. The system of claim 9 wherein the model portions are based on systems of coupled second-order differential equations that relate second derivatives of membrane potentials to sums of terms that include membrane potentials, first derivatives of membrane potentials, and multiplying constants.

11. The system of claim 7 wherein the simulator generates simulated EEG data from membrane potentials generated by the one or more functions for successive time points by left multiplying a membrane-potentials matrix that contains the membrane potentials output by the one or more functions for successive time points by a lead-field matrix that characterizes the responsiveness of each electrode or electrode pair of the first device or system to cortical and subcortical brain regions.

12. The system of claim 7 wherein the severity-level function is implemented as a convolutional neural network that outputs a severity-level probability distribution in response to input of processed simulated EEG data.

13. The system of claim 7 wherein the treatment-efficacy component outputs a treatment-efficacy value corresponding to the a severity-level probability distribution output from the severity-level function by:
selecting a severity level from the severity-level probability distribution output by the severity-level function;
computing a difference between a reference severity level and the selected severity level;
scales the computed difference to produce a treatment-efficacy value; and
outputs the treatment-efficacy value.

14. The system of claim 1 wherein the treatment-planning method uses electrical-activity data included in the received patient data and/or data recorded by the first device or system to generate a whole-brain-network model (“WBNM”) by using the received patient data and/or data recorded by the first device or system to initialize the values of multiple parameters of the computational model of the WBNM.

15. The system of claim 1 wherein the treatment-planning method modifies the computational model to ensure that the mWBNM generates simulated electrical activity that matches the electrical-activity data by:
adding adjustment terms to one or more differential equations that form a basis for the computational model; and
solving the differential equations to produce a matrix equation from which values for the adjustment terms can be determined from EEG data recorded by the first device or system and/or included in the received patient data.

16. The system of claim 1 wherein the treatment-planning method uses the mWBNM or an adjusted mWBNM to generate a treatment plan for the patient by finding a set of control-parameter values that, when a lower treatment-efficacy value indicates a more effective treatment, minimizes the treatment-efficacy value output by the mWBNM and otherwise maximized the treatment-efficacy value output by the mWBNM.

17. The system of claim 1 wherein the treatment-planning method uses the determined treatment efficacy to adjust the mWBNM by transforming the control-parameter values input to the mWBNM so that the treatment-efficacy value output by the mWBNM in response to the transformed control control-parameter values is equal to the determined treatment efficacy.

18. A method that uses a first device or system that records data from the patient, that uses a second device or system that applies a treatment to the patient according to a set of control parameters, that is implemented in one or more computer systems, and that generates a treatment plan for treating a patient, the method comprising:
receiving patient data and treatment information,
using electrical-activity data included in the received patient data and/or recorded by the first device or system to generate a whole-brain-network model (“WBNM”), based on a computational model, that simulates electrical activity in the patient's brain in response to input signals, and outputs a treatment-efficacy value corresponding to the simulated electrical activity,
generating an optimized WBNM (“WBNM*”) by adjusting model parameters of the WBNM to minimize differences between the simulated electrical activity generated by the WBNM* and the electrical-activity data,
modifying the WBNM* to generate a modified WBNM (“mWBNM”) by modifying the computational model to ensure that the mWBNM generates simulated electrical activity that matches the electrical-activity data,
using the mWBNM to generate a treatment plan for the patient, and
when limited experimentation is indicated in the received therapeutic-treatment information, carries out up to a specified number of experiments, each additional experiment carried out by
using a most recently generated treatment plan to control the second device or system to apply a treatment to the patient,
using the first device or system to record electrical-activity data, from which a treatment efficacy is determined,
using the determined treatment efficacy to adjust the mWBNM, and
using the adjusted mWBNM to generate a next treatment plan for the patient.

19. The method of claim 18 wherein the WBNM includes:
the computational model which is solved to generate one or more functions that output membrane potentials for cortical and subcortical regions of the brain at a point in time resulting from specified input signals;
a simulator that generates simulated EEG data from membrane potentials output by the one or more functions for successive time points;
a severity-level function that outputs a severity-level indication corresponding to input simulated EEG data generated by the simulator; and
a treatment-efficacy component that outputs a treatment-efficacy value corresponding to the severity-level indication output by the severity-level function.

20. The method of claim 16
wherein the treatment-planning method uses electrical-activity data included in the received patient data and/or recorded by the first device or system to generate a whole-brain-network model (“WBNM”) by using the electrical-activity data included in the received patient data and/or recorded by the first device or system to initialize the values of multiple parameters of the computational model of the WBNM;
wherein the treatment-planning method modifies the computational model to ensure that the mWBNM generates simulated electrical activity that matches the electrical-activity data by
adding adjustment terms to one or more differential equations that form a basis for the computational model, and
solving the differential equations to produce a matrix equation from which values for the adjustment terms can be determined from simulated EEG data and corresponding EEG data recorded by the first device or system and/or included in the received patient data;
wherein the treatment-planning method uses the mWBNM or an adjusted mWBNM to generate a treatment plan for the patient by finding a set of control-parameter values that, when a lower treatment-efficacy value indicates a more effective treatment, minimizes the treatment-efficacy value output by the mWBNM and otherwise maximized the treatment-efficacy value output by the mWBNM; and
wherein the treatment-planning method uses the determined treatment efficacy to adjust the mWBNM by transforming the control-parameter values input to the mWBNM so that the treatment-efficacy value output by the mWBNM in response to the transformed control control-parameter values is equal to the determined treatment efficacy.
